(19) United States
(12) Patent Application Publication
(10) Pub. No.: US 2024/0294963 A1
(43) Pub. Date: Sep. 5, 2024

(54) ENGINEERED N-GLYCOSYLTRANSFERASES WITH ALTERED SPECIFICITIES

(71) Applicant: Northwestern University, Evanston, IL (US)

(72) Inventors: Michael C. Jewett, Evanston, IL (US); Weston K. Kightlinger, Evanston, IL (US); Milan Mrksich, Evanston, IL (US); Liang Lin, Evanston, IL (US)

(21) Appl. No.: 18/005,826

(22) PCT Filed: Jul. 14, 2021
(86) PCT No.: PCT/US2021/070878

§ 371 Date: Jan. 17, 2023

Related U.S. Application Data
(60) Provisional application No. 63/051,825, filed on Jul. 14, 2020.

(51) Int. Cl. C12P 21/00; C12N 9/10

(52) U.S. Cl. CPC C12P 21/005; C12N 9/1051

(57) ABSTRACT

Disclosed are compositions, systems, and methods for synthesizing glycoproteins or recombinant glycoprotein protein in vitro and in vivo. In particular, the present invention relates to modified N-glycosyltransferases (NGTs), method for generating modified NGTs, methods for identifying and/or generating modified acceptor peptide sequences for glycosylation by NGTs, and methods for preparing glycoproteins and recombinant glycoproteins in vitro and in vivo using the modified NGTs and/or acceptor peptide sequences.

### CROSS-REFERENCE TO RELATED PATENT APPLICATIONS

The present application claims the benefit of priority under 35 U.S.C. § 119(e) to U.S. Provisional Application No. 63/051,825, filed on Jul. 14, 2020, the contents of which are incorporated herein by reference in their entireties.

### STATEMENT REGARDING FEDERALLY SPONSORED RESEARCH OR DEVELOPMENT

This invention was made with government support under 11631647 (HDTRA1-15-10052/P00001) awarded by the Defense Threat Reduction Agency and DGE-1324585 awarded by the National Science Foundation. The government has certain rights in the invention.

### FIELD

The present invention generally relates to components, systems, and methods for glycoprotein protein synthesis. In particular, the present invention relates to identification of novel N-glycosyltransferases with altered specificities and their use in synthesizing glycoproteins and recombinant glycoproteins in cells, using purified enzymes, or in cell-free protein synthesis (CFPS).

### BACKGROUND

The important roles that protein glycosylation plays in modulating the activities and efficacies of protein therapeutics have motivated the development of synthetic glycosylation systems in living bacteria and in vitro. A key challenge is the lack of glycosyltransferases that can efficiently and site-specifically glycosylate desired target proteins without the need to alter primary amino acid sequences at the acceptor site. Here, the inventors report an efficient and systematic method to engineer a library of glycosyltransferases capable of modifying comprehensive sets of acceptor peptide sequences in parallel. This approach is enabled by cell-free protein synthesis and mass spectrometry of self-assembled monolayers, and used to engineer a recently discovered prokaryotic N-glycosyltransferase (NGT). The inventors screened 26 pools of site-saturated NGT libraries to identify relevant residues that determine polypeptide specificity and then characterized 122 NGT mutants, using 1,052 unique peptides and 52,894 unique reaction conditions. The inventors define a panel of 14 NGTs that can modify 93% of all sequences within the canonical X₋₁-N-X₊₁-S/T eukaryotic glycosylation sequences as well as another panel for many non-canonical sequences (with 10 of 17 non-S/T amino acids at the X₊₂ position). The inventors then successfully applied the panel of NGTs to increase the efficiency of glycosylation for three approved protein therapeutics. This work promises to significantly expand the substrates amenable to in vitro and bacterial glycoengineering.

### SUMMARY

Disclosed are modified N-glycosyltransferases (NGTs) having enhanced glycosylation activity. In particular, the modified NGTs are capable of recognizing and glycosylating canonical and non-canonical eukaryotic target peptide sequences, and glycosylating these sequences with higher efficiency than unmodified NGTs, thereby significantly expand the substrates amenable to in vitro and bacterial glycoengineering.

### BRIEF DESCRIPTION OF THE FIGURES

FIG. 1A-D. Peptide library screening to identify ApNGT residues that determine acceptor peptide specificity. (a) The binding pocket of ApNGT (PDB ID: 3Q3H²⁹) with 26 potential peptide binding residues (red). The donor UDP binding pocket is in yellow. (b) A site-saturated variant library (SSVL) containing equal amounts of all 19 non-wildtype amino acids was synthesized as linear DNA template for each residue of the ApNGT mutant Q469A (ApQ). The A469 SSVL of ApQ is given as an example. These SSVLs were amplified by PCR to generate LETs and expressed in CFPS to produce protein SSVLs. The protein SSVLs were then used to modify a library of peptide substrates with the motif X₋₁-N-X₊₁-TRC and analyzed via SAMDI-MS. A heatmap of peptide modification is shown (bottom-right), with the same descending order of average modification for amino acids at the X₋₁ and X₊₁ positions, respectively, as ApQ. A new heatmap of −ln(1−Y) (bottom-left), where Y is the peptide modification, was generated. Because the inventors use the concentration of peptides much lower than K_{M}, the average k_{cat}/K_{M} of each SSVL can be calculated using the average −ln(1−Y), (0.36 in bold, grey square) in heatmap, with the equation k_{cat} K_{M}−ln(1−Y)/c/t. The average k_{cat}/K_{M} relative to ApQ is shown in (c). All SSVLs show decreased average k_{cat}/K_{M}. (d) The average value of −ln(1−Y) in each row of X₋₁ amino acid, and each column of X₊₁ amino acid for each SSVL, is compared to that of ApQ, which chooses to have the same value of average −ln(1−Y) of the entire library as the SSVL, to show the percentage difference using the equation 2*|Ave(X)−Ave(ApQ)|/(Ave(X)+Ave(ApQ)). Mean percentage difference of the X₋₁ (left) and X₊₁ (right) positions are presented using the average of the percentage differences for all rows and columns, respectively. Values higher than 20% and 30% are highlighted in light grey bars and arrows, respectively. All experiments were completed with n=1.

FIG. 2A-C. Screening individual ApQ mutants with unique specificities for the X₋₁ and X₊₁ acceptor peptide positions. (a) The relative average k_{cat}/K_{M} of individual mutants, from T438, A469, and H219, compared to ApQ against the X₋₁-N-X₊₁-TRC peptide library. Only T438S shows a slight increase in relative activity (1.1-fold), while T438D/E/K/R/W and H219R show poor activities that are less than 0.001-fold of ApQ (H219R was not screened with entire library and T438D/E/K/R/W were screened but showed poor modification, see FIG. 22). (b) Mean percentage differences of X₋₁ (upper), X₊₁ (middle), and entire library (down) for each mutant compared to ApQ. The mean percentage difference of the entire library is the average of the value of X₋₁ and X₊₁. A value higher than 75% is indicated by arrows. (c) Heatmap of the relative selectivity for amino acids at the X₋₁ and X₊₁ positions of ApQ and all individual mutants of the three selected residues (T438, A469, and H219). Relative specificity is defined as the ratio of the average −ln(1−Y) at each amino acid lane to the maximum value of all 19 X₋₁ or X₊₁ lanes. The amino acids at the X₋₁ and X₊₁ positions are organized in the same order as modification heatmaps, and the order of individual mutants at each residue is the same as a and b. The heatmap with numerical values is also shown at FIG. 25. These data clearly show that T438 mutants exhibit large specificity differences for the X₋₁ position, H219F/W for X₊₁, and A469 mutants for both X₋₁ and X₊₁. All experiments were completed with n=1.

FIG. 3A-D. Expanded set of peptide sequences eligible for glycosylation by engineered NGTs. (a-b) Modification heatmaps of the peptide libraries X₋₁-N-X₊₁-T (a) or X₋₁-N-X₊₁-S (b) for ApQ (left) and the maximum modification from 14 selected NGTs (ApQ, H219F, H219W, T438S, T439E, A469G, A469I, H495D, H219F-T438S, H219F-H495D, H219W-T438S, H219W-H495D, A469G-H495D, and A469I-H495D) (right). All NGTs were tested using the same condition: 0.545 μM NGT produced in LET-CFPS, 30° C. for 3 h. There are significant improvements for the peptides inefficiently modified by ApQ. Specifically, peptides with Asn, Asp at the X₊₁ position, and Lys, Arg at X₋₁ are modified at much higher efficiencies by the panel of 14 NGTs compared to ApQ alone. Heatmaps annotated with numerical values and the optimal NGT for each peptide substrate is shown in FIG. 32. All experiments were completed with n=1. (c-d) Comparison of all peptide substrates within the canonical glycosylation motif (X₋₁-N-X₊₁-T, X₊₁≠P in c) or X₋₁-N-X₊₁-S(X₊₁≠P in d) modified with more than 80% efficiency in a-b by ApQ (left) and the maximum value from 14 selected NGTs (right). The percentage of peptides with more than 80% modification (highlighted in blue) increased from 56% to 80% for the X₋₁-N-X₊₁-T library and from 33% to 51% for the X₋₁-N-X₊₁-S library.

FIG. 4A-C. Selected NGT mutants enabling superior modification of therapeutic proteins. (a) Peptides with the sequences from approved therapeutic proteins are confirmed to have greater modification when glycosylated by purified mutants compared to ApQ. Folds of modification to ApQ are presented for each peptide. All experiments were completed with n=3 IVG reactions. Experimental conditions: 1 μM (for TNYS), 0.05 μM (for LNLS), or 0.2 μM (for YNST) purified NGT, 30° C. for 3 h. In the graph, ApQ and A469I are the first set of bars: ApQ and T438S are the second set of bars; ApQ and H495 are the third set of bars. (b) Purified approved therapeutic proteins exhibit enhanced modifications when glycosylated by selected mutants compared to ApQ. Folds of modification to ApQ are presented for each protein glycosylation site. After IVG, the solutions were dialyzed, trypsinized, and analyzed with LC-qTOF. ApQ showed no detectable modification of IFNγ (marked as “ND”). All experiments were completed with n=2 or n=3 individual IVG reactions (as indicated in graph). Experimental condition: 5 μM purified NGT, 5 mM UDP-Glc, 30° C. for 12 h. In the graph, A469I is the first bar; ApQ is the second bar; and T438S is the third bar. (c) Fc showed increased modification for H495D over ApQ in CFPS expression. Folds of modification to ApQ are presented. Fc, with a 6×His tag, was expressed by LET-CFPS supplemented with purified NGT and UDP-Glc. After the CFPS reaction, Fc was purified with magnetic beads, dialyzed, trypsinized, and analyzed with LC-qTOF. In the graph, ApQ is the first bar and H495D is the second bar. All experiments were completed with n=3 individual CFPS reactions. Experimental condition: 2 μM purified NGT, 5 mM UDP-Glc, 30° C. LET-CFPS for 6 h. All protein modification efficiencies can be found in FIG. 12. All p values were from two-tailed t-test with p<0.01 (**) or p<0.001 (***).

FIG. 5A-B. Screening NGT mutants for expanded specificity at the X₊₂ position. (a) Average −ln(1−Y) heatmap for non-S/T amino acids at X₊₂ with all R177 and D215 individual mutants (values for T/S shown below). Six X₋₁-N-X₊₁-T peptide sequences preferred by ApQ were substituted with 18 amino acids (Cys excluded) at X₊₂ and screened against all individual R177 and D215 mutants. All heatmaps show results from n=1 experiment. All reactions were performed with 0.545 μM NGTs produced in LET-CFPS, 30° C. for 12 h. X₊₂ amino acid lanes are arranged in the same descending order as ApQ shown in FIG. 18. The modification heatmaps for X₊₂ with ApQ and all individual mutants are shown in FIG. 36. (b) The relative amino acid selectivity of X₊₂ based on data in a, divided by the maximum value of all X₊₂ (except S/T) for each mutant. The relative selectivity for S/T is also shown below over the same maximum value and may be higher than 100%. When Asn is present at X₊₂, the modification may come from the second Asn at NRC, rather than N-X-N if the modification for W-N-I/V-N-RC is more preferred than A-N-I/V-N-RC.

FIG. 6. Provides a table listing the strains and plasmids used in Example 1.

FIG. 7. Provides a table showing single and double mutants of ApQ used in Example 1.

FIG. 8. Provides a table showing average relative ionization factors (RIFs) of peptide libraries used in Example 1. For each peptide library, six to twelve representative peptides were analyzed to calculate the average RIF. Peptide modifications were calculated according to the formula I(F)/(I(S)*RIF+I(P)), in which I(P) is intensity of glycosylated peptides in mass spectra, I(S) is intensity of aglycosylated peptides in mass spectra.

FIG. 9. Provides a table showing reaction conditions for SSVLs and individual single mutants with peptide libraries. Indicated concentrations of NGTs, produced in LET-CFPS, were reacted with 50 μM peptides and 2.5 mM UDP-Glc in 100 mM HEPES buffer (pH 8) and 500 mM NaCl. The reactions were incubated at 30° C. for indicated reaction times.

FIG. 10. Provides a table showing peptide sequences discovered in this work that are glycosylated with substantially higher efficiency by ApQ mutants under identical reaction conditions. In total, 227 peptides from the 722-member X₋₁NX₊₁S/TRC peptide library were glycosylated at least 20% more efficiently by selected NGT mutants compared to ApQ. Reaction condition: 0.545 μM NGT produced in LET-CFPS, 30° C. for 3 h. “Y” represents modification efficiency by selected mutant, “Y by ApQ” represents efficiency by ApQ, and “ΔY” represents the increase in modification observed when treated with the indicated ApQ mutant compared to ApQ.

FIG. 11. Provides a table showing peptide sequences discovered in this work which exhibit substantially higher approximate k_{cat}/K_{M} with the mutants. From each X₋₁NX₊₁TRC peptide library modified by ApQ and selected mutants with different reaction conditions, the inventors calculated the approximate k_{cat}/K_{M} for each peptide (see Methods). In total, 33 of the peptides, glycosylated with >75% efficiency by ApQ in FIG. 3a, were found to exhibit more than two-fold activity with ApQ mutants developed in this study compared to ApQ.

FIG. 12. Provides a table showing results of LC-qTOF analysis of peptides from trypsin treated glycosylated protein therapeutics. Peptide species that were not observed (i.e., not detected) are marked with “-”. In the section labeled “purified”, 10 μM purified protein substrate was reacted with 5 μM purified enzyme at 30° C. for 12 h. In the “CFPSe” section, Fc was produced in LET-CFPS at 30° C. for 6 h in the presence of purified enzyme at concentrations of 2 μM ApQ, 2 μM H495D, or 5 μM H495D (marked as H495D′). For the Fc samples under the “After CFPS” section, Fc was first produced in LET-CFPS at 30° C. for 20 h and then supplemented with 2 μM purified ApQ and incubated at 30° C. for 6 h.

FIG. 13. Provides a table showing primers used for PCR mutagenesis. Overlapped sequences are denoted in by underline, with the mutation site highlighted in bold, and used to calculate Tm₁. The extended sequences are in italics and was used to calculate Tm₂ (see Methods).

FIG. 14A-E. LET-CFPS reactions express ApQ, SSVLs, and individual mutants at similar levels. (A) DNA gel of ApQ and 26 SSVL linear expression templates amplified by PCR. This linear expression template contains the coding sequence as well as a promoter and terminator. A band at approximately 2.2 kb was observed in all lanes indicating amplification of ApQ and SSVLs. PCR products were directly used for LET-CFPS of NGTs. (B) SDS-PAGE of soluble CFPS fractions. ApQ and all 26 SSVLs were expressed by LET-CFPS in E. coli BL21 Star (DE3) lysates at similar levels. (C) SDS-PAGE of soluble CFPS fractions for ApQ and 19 individual T438 mutants. NGT mutants were expressed at similar levels. SfGFP was used as a control. (D) An autoradiogram representative of n=2 experiments with similar results confirmed that the CFPS reactions primarily produced equal full-length NGTs without large truncations in soluble fragments. The autoradiogram was generated by a 48-h exposure of an SDS-PAGE gel for NGTs produced in CFPS with ¹⁴C-leucine. All SDS-PAGE gels in B-D used 4-12% Bis-tris gels run with MOPS buffer at 150 V and a SeeBlue Plus2 prestained ladder followed by staining with InstantBlue Coomassie stain (Expedeon). (E) Total and soluble yields of NGTs from CFPS reactions were determined using ¹⁴C-leucine incorporation (n=3 experiments). Equal expression levels for all NGTs were observed in total as well as soluble fractions. All CFPS reactions were incubated for 20 h at 22° C. Soluble fractions in A-E were isolated after centrifugation at 12,000×g for 15 min at 4° C. The inventors used the average concentration of 10.9 μM for all NGTs expressed in LET-CFPS, including ApQ, SSVLs, as well as individual single and double NGT mutants.

FIG. 15. Modification efficiency heatmaps for SSVLs screened across an X₋₁-N-X₊₁-TRC peptide library. All X₋₁ and X₊₁ amino acid lanes in X₋₁NX₊₁TRC and X₋₁NX₊₁SRC library heatmaps are arranged in the same descending order of average modification from left to right and top to bottom observed in the X₋₁NX₊₁TRC library heatmap modified by 0.0218 μM ApQ (FIG. 16). All experiments were conducted with n=1. Reaction conditions are shown in FIG. 9.

FIG. 16A-D. ApQ reference heatmaps and calculation of peptide selectivity percentage differences. (A) Six concentrations of ApQ, synthesized in CFPS, were reacted with the peptide library X₋₁-N-X₊₁-TRC, at 30° C. for 1 h to generate reference heatmaps with various average −ln(1−Y) values for calculation of percentage differences between mutants and ApQ (see below and Methods). After the reaction, the glucose modifications were analyzed and the modification heatmaps were generated. All X₋₁ and X₊₁ amino acid lanes are arranged the same as the descending order of average modification from left to right and top to bottom observed in the X₋₁NX₊₁TRC library heatmap modified by 0.0218 μM ApQ. This same order was used for all X₋₁NX₊₁TRC and X₋₁NX₊₁SRC heatmaps in this work. (B) The corresponding ApQ reference heatmaps with values of −ln(1−Y), where Y is the modification yield. (C) Sample calculation of percentage difference between A469X and ApQ. Linear assumption was made between heatmaps of two close concentrations. The average −ln(1−Y) value for X₋₁NX₊₁TRC library modified by A469X was 0.36, between the value of the reference ApQ heatmaps with 0.0142 μM ApQ (0.23) and 0.0218 μM ApQ (0.39). The inventors then used linear interpolation of these two reference ApQ heatmaps to generate a theoretical heatmap for ApQ with an average −ln(1−Y) of 0.36. The inventors then calculated the percentage difference between the average −ln(1−Y) value of each X₋₁ and X₊₁ lane in this theoretical ApQ heatmap and the A469X heatmap. The mean percentage difference of all X₋₁ lanes is 0.29 and that of all X₊₁ lanes is 0.50. (D) Dependence of average k_{cat}/K_{M} on average −ln(1−Y) of ApQ based on reference heatmaps. When computing the relative k_{cat}/K_{M} of each mutant (or SSVL) compared to ApQ, the inventors also adjusted the k_{cat}/K_{M} for ApQ to match the average −ln(1−Y) of the mutant heatmap using linear interpolation. All experiments were conducted with n=1.

FIG. 17. Mean percentage differences for any of the 26 SSVLs and ApQ compared to each other, for X₋₁ (top) and X₊₁ (bottom). Based on X₋₁NX₊₁TRC heatmaps shown in FIG. 15, the inventors calculated the mean percentage difference between any two of 26 SSVLs and ApQ from the mean percentage difference between each SSVL and ApQ (see Methods). Values higher than 20% and 40% are highlighted in blue and red, respectively. The SSVLs are arranged in descending order of the average difference to all others. T438X and A469X exhibited the greatest changes in specificity compared to all other SSVLs. Based on their location in the crystal structure, the inventors believe that both T438 and A469 interact with the X₋₁ position of the acceptor peptide. The inventors then concluded that R177, M218 and H219 are likely interacting with the X₊₁ position of the acceptor peptide. While H214 also effects specificity, its location in the crystal structure compared to other putative binding residues does not support direct interaction with the X₊₁ position of the peptide.

FIG. 18. SSVL screening to determine important residues for X₊₂ specificity. SSVLs identified as candidates for interaction with the X₊₂ position of the acceptor peptide based on the crystal structure and possible binding residues for X₋₁ and X₊₁, were screened across an (X₋₁NX₊₁)X₊₂RC library. All X₊₂ amino acid lanes are arranged in the same descending order as ApQ. SSVLs of residues known to interact with the X₋₁ position of the acceptor peptide, T438X and A469X, were also screened as negative controls. R177X and D215X exhibits preferences for other amino acids at X₊₂ besides canonical S/T. Based on the data, the crystal structure, and the importance of S/T at X₊₂ for modification, the inventors hypothesize that D215 forms a hydrogen bond with S/T at X₊₂. While the inventors are uncertain of how R177 interacts with the X₊₂ position, it indicates that R177 plays an important role for determining enzyme specificity. All experiments were conducted with n=1. Reaction conditions are shown in FIG. 9.

FIG. 19. SSVL screening to determine important residues for X₋₂ specificity. SSVLs identified as candidates for interaction with the X₋₂ position of the acceptor peptide based on the crystal structure and possible binding residues for X₋₁ and X₊₁, were screened across an X₋₂(X₋₁NX₊₁)TRC library. All X₋₂ amino acid lanes are arranged in the same descending order as ApQ. H277X shows increased modification of peptides with Ile and Gln and decreased with Pro at X₋₂. H277X also shows decreased modification when there is a Trp residue at X₋₁, which is in accordance with the results for H277X in X₋₁NX₊₁TRC screening (FIG. 15). All experiments were conducted with n=1. Reaction conditions are shown in FIG. 9.

FIG. 20. SSVL screening to determine important residues for X₊₃ specificity. SSVLs identified as candidates for interaction with the X₊₃ position of the acceptor peptide based on the crystal structure and possible binding residues for X₊₁ and X₊₂, were screened across an (X₋₁NX₊₁)TX₊₃RC library. All X₊₃ amino acid lanes are arranged in the same descending order as ApQ. H214X shows increased modification on peptides with Pro, Asp and Glu at X₊₃. R177X also shows increased modification on peptides with Arg, Lys and His at X₊₃, which may be the electrostatic repulsion between these amino acids and residue R177. All experiments were conducted with n=1. Reaction conditions are shown in FIG. 9.

FIG. 21. Hypothesized peptide binding residues based on screening results. Based on the ApNGT crystal structure and the screening results at FIGS. 15, and 17-20, the inventors propose the following interactions between enzyme residues and positions of the acceptor peptide: H277 binds to the X₋₂; T438 and A469 binds to X₋₁; R177, M218 and H219 bind to X₊₁; R177 and D215 bind to X₊₂; H214 binds to X₊₃. Another residue which affects selectivity for K/R at X₋₁, H495, is also shown (data shown in FIG. 29A-C). While further structural studies will be required to confirm these interactions, this provides a model for understanding which residues will most heavily influence the specificity of NGTs. This is based on the structure of ApNGT² (PDB #3Q3H), so the residue 469 is Gln in the figure. SSVLs identified as candidates for interaction with the X₊₃ position of the acceptor peptide based on the crystal structure and possible binding residues for X₊₁ and X₊₂, were screened across an (X₋₁NX₊₁)TX₊₃RC library. All X₊₃ amino acid lanes are arranged in the same descending order as ApQ. H214X shows increased modification on peptides with Pro, Asp and Glu at X₊₃. R177X also shows increased modification on peptides with Arg, Lys and His at X₊₃, which may be the electrostatic repulsion between these amino acids and residue R177. All experiments were conducted with n=1. Reaction conditions are shown in FIG. 9.

FIG. 22. Modification efficiency heatmaps for individual T438 mutants screened across an X₋₁-N-X₊₁-TRC peptide library. All experiments were conducted with n=1. Reaction conditions are shown in FIG. 9.

FIG. 23. Modification efficiency heatmaps for individual A469 mutants screened across an X₋₁-N-X₊₁-TRC peptide library. All experiments were conducted with n=1. Reaction conditions are shown in FIG. 9.

FIG. 24. Modification efficiency heatmaps for individual H219 mutants screened across an X₋₁-N-X₊₁-TRC peptide library. All experiments were conducted with n=1. Reaction conditions were shown in FIG. 9.

FIG. 25. Relative selectivity of ApQ and individual T438, A469 and H219 mutants for amino acids at X₋₁ and X₊₁ positions annotated with numerical values. FIG. 2C shows this heatmap without numerical values.

FIG. 26. Mean percentage difference heatmaps of individual T438 (top), A469 (middle) and H219 (bottom) mutants, as well as ApQ, across the full X₋₁NX₊₁TRC library. The inventors calculated the mean percentage difference between any two NGTs for T438 (top), A469 (middle) and H219 (bottom) mutants, as well as ApQ, across the full X₋₁NX₊₁TRC library (see Methods). The mutants are arranged in the descending order of average difference to all others. Values larger than 0.40 and 0.80 are highlighted in blue and red, respectively. T438H/L/Q/P/F/Y/N/G, A469E/P/R/Y/N/H/F/D/G/M/L/K, and H219W/F have higher average differences than ApQ.

FIG. 27. Modification efficiency heatmaps for ApQ and selected individual mutants across a X₋₁NX₊₁TRC peptide library. All experiments were conducted with n=1. Reaction conditions: 0.545 μM CFPS NGT, 30° C. for 3 h.

FIG. 28. Modification efficiency heatmaps for ApQ and selected individual mutants across a X₋₁NX₊₁SRC peptide library. All experiments were conducted with n=1. Reaction conditions: 0.545 μM CFPS NGT, 30° C. for 3 h.

FIG. 29A-C. Identifying mutants with increased specificity towards peptides with Lys or Arg at X₋₁. (A) SSVLs that showed relative high activity and had mutated residues predicted to be nearby the X₋₁ position, were screened with K/R-N-X₊₁-TRC for increased modification. H495X and T439X showed more modification than ApQ for some peptides, highlighted in red. (B) The glycosylation increase for the T439X SSVL with peptides K/R-N-Y-TRC was mainly from T439D/E mutants. All 19 individual H495 mutants were screened with the peptides. H495D significantly improved the modification for most peptides. (C) Mutants showing increased modification in B were screened with K/R-N-X₊₁-SRC. All experiments were conducted with n=1. All reaction conditions: 0.545 μM CFPS NGT, 30° C. for 3 h.

FIG. 30. Modification efficiency heatmaps with entire X₋₁NX₊₁TRC library for representative mutants, T439E, H495D and H495Q. T439E and H495D show increased selectivity across peptides with K/R at X₋₁, while H495Q did not. All experiments were conducted with n=1. Reaction conditions are shown in FIG. 9.

FIG. 31A-B. Modification efficiency heatmaps for double mutants. (A) Four double mutants with H495D were screened with K/R-N-X₊₁-S/T-RC. (B) Four double mutants with H219F/W were screened with X₋₁-N-N/D-S/T-RC. All experiments were conducted with n=1. All reaction conditions: 0.545 μM CFPS NGT, 30° C. for 3 h.

FIG. 32. Map of optimal NGT mutants for each canonical glycosylation sequence. This peptide map shows the maximum modification efficiency achieved by ApQ and 13 selected single or double mutants discovered in this work (listed at center) across canonical glycosylation sequences (X₋₁NX₊₁SRC and X₋₁NX₊₁TRC). This is the same data found in FIG. 3a-b and was derived from FIGS. 27, and 29-31. Modifications greater than 0.05 are highlighted in gray and regarded as sequences which can be modified (93% of the canonical sequences). The peptide modification values are color-coded by the NGT that yielded the maximum modification.

FIG. 33A-C. LC-qTOF MS/MS of targeted tryptic peptides within approved therapeutic proteins. This peptide sequencing by MS/MS confirms the identity of tryptic peptides, as well as the glycopeptides with nearly the same MS/MS spectra. Extracted ion chromatograms of MS1 of these tryptic peptides was used for quantification of glycosylation in FIG. 4. (A) IFNγ target peptides: LTNYSVTDLNVQR, +2 charged m/z of 761.90 in MS1; Glc-peptide, +2 charged m/z of 842.92 in MS1. (B) GM-CSF target peptides: LLNLSR, +1 charged m/z at 715.45 in MS1; Glc-peptide, +1 charged m/z of 877.50 in MS1. (C) Fc target peptides: EEQYNSTYR, +2 charged m/z at 595.26 in MS1; Glc-peptide, +2 charged m/z of 676.29 in MS1. A collision energy of 50 eV was used.

FIG. 34A-B. Fc expression in LET-CFPS with pre-existing NGTs or NGTs added after the CFPS. (A) After 20-h expression of Fc in LET-CFPS and centrifugation to isolate the soluble fraction, 2 μM purified ApQ and 5 mM UDP-Glc were added, and the reaction was incubated at 30° C. for 6 h. 15% modification was achieved compared to 46% when purified ApQ was added at the beginning of the CFPS reaction (shown in FIG. 4c and FIG. 12). (B) With 5 μM purified H495D and 5 mM UDP-Glc present during LET-CFPS expression of Fc at 30° C. for 6 h, Fc was modified at 80%. All experiments were completed with n=2 individual IVG reactions.

FIG. 35. Modification efficiency heatmaps for individual R177 and D215 mutants across (X₋₁NX₊₁)X₊₂RC peptide library. The X₊₁ amino acids are arranged in the same descending order as FIG. 18. All experiments were conducted with n=1. Reaction condition: 0.545 μM CFPS NGT, 30° C. for 12 h.

FIG. 36. Modification efficiency heatmaps for individual R177 mutants across an X₋₁-N-X₊₁-TRC peptide library. R177 individual mutants showed differences in X₋₁ and X₊₁ selectivity. This data was used to choose the X₋₁ and X₊₁ combinations with unique X₊₂ preferences for each mutant. All experiments were conducted with n=1. Reaction conditions are shown in FIG. 9.

FIG. 37. Percentage intensity heatmap for ApQ and four highly active mutants screened with UDP-GlcN and six representative peptides. T438S and A469I showed higher GlcN modification than ApQ on some peptides. All experiments were conducted with n=1. Reaction condition: 1.09 μM NGT produced in LET-CFPS, 2.5 mM UDP-GlcN, 30° C. for 12 h.

FIG. 38. Provides DNA sequences encoding NGT's expressed by LET-CFPS, NGT's expressed and purified from E. coli, and substrate proteins expressed in LET-CFPS. Key: TRANSLATED REGION (all caps); MUTANT SITES (underlined caps); untranslated region (lower case); T7 promoter (underlined lowercase); T7 terminator (italics, lowercase).

FIG. 39. Provides NGT amino acid sequences of the following organisms: Salmonella enterica; Kingella kingae, Aggregatibacter aphrophilus; Burkholderia sp; Bibersteinia trehalosi; Escherichia coli; Haemophilus ducreyi; Mannheimia haemolytica; Haemophilus influenzae; Yersinia enterocolitia; Yersinia pestis; and Actinobacillius pleuropneumoniae.

FIG. 40. Provides a CLUSTAL OMEGA alignment of the NGT amino acid sequences of the following organisms: Kingella kingae; Haemophilus influenzae; Aggregatibacter aphrophilus; Mannheimia haemolytica; Bibersteinia trehalosi; Haemophilus ducreyi; Actinobacillius pleuropneumoniae; Burkholderia sp; Yersinia enterocolitia; Yersinia pestis; Salmonella enterica; and Escherichia coli.

FIG. 41. Provides a table showing protein sequence identity percentages. The percent amino acid identity among Kingella kingae, Haemophilus influenza, Aggregatibacter aphrophilus, Mannheimia haemolytica, Bibersteinia trehalosi, Haemophilus ducreyi, and Actinobacillius pleuropneumoniae, shown in the upper left portion of the table, ranges between 62.5% and 76.25%.

FIG. 42A-B. Provides a structural alignment generated by the PHYRE2 protein fold recognition Engine (http://www.sbg.bio.ic.ac.uk/˜phyre2/html/page.cgi?id=index) showing that Escherichia coli NGT (EcNGT), Aggregatibacter aphrophilus NGT (AaNGT), and Actinobacillius pleuropneumoniae NGT (ApNGT) (which are found to have diverse sequences) actually have a similar structure, and therefore could be engineered in a similar fashion to ApNGT. PHYRE2 uses all currently known crystal structures to predict the most likely fold of uncrystallized proteins based on their sequences. A) shows the alignment of EcNGT (red) and APNGT (blue). UDP ligand in the active site is shown in green spheres. B) shows the alignment of AaNGT (red) and APNGT (blue). UDP ligand in active site shown as green spheres.

FIG. 43. Provides CLUSTAL OMEGA alignment of the amino acid sequences of the following organisms: Kingella kingae, Haemophilus influenza, Aggregatibacter aphrophilus, Mannheimia haemolytica, Bibersteinia trehalosi, Haemophilus ducreyi, and Actinobacillius pleuropneumoniae.

FIG. 44A-B. Provides a structural alignment generated by the PHYRE2 protein fold recognition Engine, showing that Kingella kingae NGT (KkNGT), Mannheimia haemolytica (MhNGT), and ApNGT actually have a similar structure, and therefore could be engineered in a similar fashion to ApNGT. Among the NGT sequences provided in FIG. 43, KkNGT is the most divergent from ApNGT; MhNGT is middle divergent. A) shows the alignment of KkNGT (red) and ApNGT (cyan). UDP ligand in active site shown in green spheres. B) shows the alignment of MhNGT (red) and APNGT (cyan). UDP ligand in active site is shown as green spheres.

FIG. 45. Provides CLUSTAL OMEGA alignment of the NGT amino acid sequences of the following organisms: Mannheimia haemolytica (MH), Haemophilus ducreyi (HD), and Actinobacillius pleuropneumoniae (AP). Amino acids in bold font correspond to ApNGT amino acids F39, R177, H214, D215, M218, H219, Y222, H272, H277, S278, 1279, R281, M349, G370, H371, T438, T439, M440, K441, Q469, H495, P497, Y498, F517, N521, D525.

FIG. 46A-B. Provides a structural alignment generated by the PHYRE2 protein fold recognition Engine, showing that Haemophilus ducreyi NGT (HdNGT), Mannheimia haemolytica NGT (MhNGT) and ApNGT have a similar structure, and therefore could be engineered in a similar fashion to ApNGT. A) shows the alignment of HdNGT (red) to ApNGT (cyan). UDP ligand in the active site is shown by green spheres. B) shows the alignment of MnNGT (red) to ApNGT (cyan). UDP ligand in the active site is shown as green spheres.

### DETAILED DESCRIPTION

### Definitions and Terminology

The disclosed components, systems, and methods for glycoprotein and recombinant glycoprotein protein synthesis may be further described using definitions and terminology as follows. The definitions and terminology used herein are for the purpose of describing particular embodiments only, and are not intended to be limiting.

As used in this specification and the claims, the singular forms “a,” “an,” and “the” include plural forms unless the context clearly dictates otherwise. For example, the term “a oligosaccharide” or “an N-glycosyltransferase” should be interpreted to mean “one or more oligosaccharides” and “one or more N-glycosyltransferase,” respectively, unless the context clearly dictates otherwise. As used herein, the term “plurality” means “two or more.”

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean up to plus or minus 10% of the particular term and “substantially” and “significantly” will mean more than plus or minus 10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion of additional components other than the components recited in the claims. The term “consisting essentially of” should be interpreted to be partially closed and allowing the inclusion only of additional components that do not fundamentally alter the nature of the claimed subject matter.

The phrase “such as” should be interpreted as “for example, including.” Moreover the use of any and all exemplary language, including but not limited to “such as”, is intended merely to better illuminate the invention and does not pose a limitation on the scope of the invention unless otherwise claimed.

Furthermore, in those instances where a convention analogous to “at least one of A, B and C, etc.” is used, in general such a construction is intended in the sense of one having ordinary skill in the art would understand the convention (e.g., “a system having at least one of A, B and C” would include but not be limited to systems that have A alone, B alone, C alone, A and B together, A and C together, B and C together, and/or A, B, and C together.). It will be further understood by those within the art that virtually any disjunctive word and/or phrase presenting two or more alternative terms, whether in the description or figures, should be understood to contemplate the possibilities of including one of the terms, either of the terms, or both terms. For example, the phrase “A or B” will be understood to include the possibilities of “A” or ‘B or “A and B.”

All language such as “up to,” “at least,” “greater than,” “less than,” and the like, include the number recited and refer to ranges which can subsequently be broken down into ranges and subranges. A range includes each individual member. Thus, for example, a group having 1-3 members refers to groups having 1, 2, or 3 members. Similarly, a group having 6 members refers to groups having 1, 2, 3, 4, or 6 members, and so forth.

The modal verb “may” refers to the preferred use or selection of one or more options or choices among the several described embodiments or features contained within the same. Where no options or choices are disclosed regarding a particular embodiment or feature contained in the same, the modal verb “may” refers to an affirmative act regarding how to make or use and aspect of a described embodiment or feature contained in the same, or a definitive decision to use a specific skill regarding a described embodiment or feature contained in the same. In this latter context, the modal verb “may” has the same meaning and connotation as the auxiliary verb “can.”

As used herein, the terms “bind,” “binding,” “interact,” “interacting,” “occupy” and “occupying” refer to covalent interactions, noncovalent interactions and steric interactions. A covalent interaction is a chemical linkage between two atoms or radicals formed by the sharing of a pair of electrons (a single bond), two pairs of electrons (a double bond) or three pairs of electrons (a triple bond). Covalent interactions are also known in the art as electron pair interactions or electron pair bonds. Noncovalent interactions include, but are not limited to, van der Waals interactions, hydrogen bonds, weak chemical bonds (via short-range noncovalent forces), hydrophobic interactions, ionic bonds and the like. A review of noncovalent interactions can be found in Alberts et al., in Molecular Biology of the Cell, 3d edition, Garland Publishing, 1994. Steric interactions are generally understood to include those where the structure of the compound is such that it is capable of occupying a site by virtue of its three dimensional structure, as opposed to any attractive forces between the compound and the site.

### Polynucleotides and Synthesis Methods

The terms “nucleic acid” and “oligonucleotide,” as used herein, refer to polydeoxyribonucleotides (containing 2-deoxy-D-ribose), polyribonucleotides (containing D-ribose), and to any other type of polynucleotide that is an N glycoside of a purine or pyrimidine base. There is no intended distinction in length between the terms “nucleic acid”, “oligonucleotide” and “polynucleotide”, and these terms will be used interchangeably. These terms refer only to the primary structure of the molecule. Thus, these terms include double- and single-stranded DNA, as well as double- and single-stranded RNA. For use in the present methods, an oligonucleotide also can comprise nucleotide analogs in which the base, sugar, or phosphate backbone is modified as well as non-purine or non-pyrimidine nucleotide analogs.

Oligonucleotides can be prepared by any suitable method, including direct chemical synthesis by a method such as the phosphotriester method of Narang et al., 1979, Meth. Enzymol. 68:90-99; the phosphodiester method of Brown et al., 1979, Meth. Enzymol. 68:109-151; the diethylphosphoramidite method of Beaucage et al., 1981, Tetrahedron Letters 22:1859-1862; and the solid support method of U.S. Pat. No. 4,458,066, each incorporated herein by reference. A review of synthesis methods of conjugates of oligonucleotides and modified nucleotides is provided in Goodchild, 1990, Bioconjugate Chemistry 1(3): 165-187, incorporated herein by reference.

The term “amplification reaction” refers to any chemical reaction, including an enzymatic reaction, which results in increased copies of a template nucleic acid sequence or results in transcription of a template nucleic acid. Amplification reactions include reverse transcription, the polymerase chain reaction (PCR), including Real Time PCR (see U.S. Pat. Nos. 4,683,195 and 4,683,202; PCR Protocols: A Guide to Methods and Applications (Innis et al., eds, 1990)), and the ligase chain reaction (LCR) (see Barany et al., U.S. Pat. No. 5,494,810). Exemplary “amplification reactions conditions” or “amplification conditions” typically comprise either two or three step cycles. Two-step cycles have a high temperature denaturation step followed by a hybridization/elongation (or ligation) step. Three step cycles comprise a denaturation step followed by a hybridization step followed by a separate elongation step.

The terms “target,” “target sequence”, “target region”, and “target nucleic acid,” as used herein, are synonymous and refer to a region or sequence of a nucleic acid which is to be amplified, sequenced, or detected.

The term “hybridization,” as used herein, refers to the formation of a duplex structure by two single-stranded nucleic acids due to complementary base pairing. Hybridization can occur between fully complementary nucleic acid strands or between “substantially complementary” nucleic acid strands that contain minor regions of mismatch. Conditions under which hybridization of fully complementary nucleic acid strands is strongly preferred are referred to as “stringent hybridization conditions” or “sequence-specific hybridization conditions”. Stable duplexes of substantially complementary sequences can be achieved under less stringent hybridization conditions; the degree of mismatch tolerated can be controlled by suitable adjustment of the hybridization conditions. Those skilled in the art of nucleic acid technology can determine duplex stability empirically considering a number of variables including, for example, the length and base pair composition of the oligonucleotides, ionic strength, and incidence of mismatched base pairs, following the guidance provided by the art (see, e.g., Sambrook et al., 1989, Molecular Cloning—A Laboratory Manual, Cold Spring Harbor Laboratory, Cold Spring Harbor, New York; Wetmur, 1991, Critical Review in Biochem. and Mol. Biol. 26(3/4):227-259; and Owczarzy et al., 2008, Biochemistry, 47: 5336-5353, which are incorporated herein by reference).

The term “primer,” as used herein, refers to an oligonucleotide capable of acting as a point of initiation of DNA synthesis under suitable conditions. Such conditions include those in which synthesis of a primer extension product complementary to a nucleic acid strand is induced in the presence of four different nucleoside triphosphates and an agent for extension (for example, a DNA polymerase or reverse transcriptase) in an appropriate buffer and at a suitable temperature.

A primer is preferably a single-stranded DNA. The appropriate length of a primer depends on the intended use of the primer but typically ranges from about 6 to about 225 nucleotides, including intermediate ranges, such as from 15 to 35 nucleotides, from 18 to 75 nucleotides and from 25 to 150 nucleotides. Short primer molecules generally require cooler temperatures to form sufficiently stable hybrid complexes with the template. A primer need not reflect the exact sequence of the template nucleic acid, but must be sufficiently complementary to hybridize with the template. The design of suitable primers for the amplification of a given target sequence is well known in the art and described in the literature cited herein.

Primers can incorporate additional features which allow for the detection or immobilization of the primer but do not alter the basic property of the primer, that of acting as a point of initiation of DNA synthesis. For example, primers may contain an additional nucleic acid sequence at the 5′ end which does not hybridize to the target nucleic acid, but which facilitates cloning or detection of the amplified product, or which enables transcription of RNA (for example, by inclusion of a promoter) or translation of protein (for example, by inclusion of a 5′-UTR, such as an Internal Ribosome Entry Site (IRES) or a 3′-UTR element, such as a poly(A)ₙ sequence, where n is in the range from about 20 to about 200). The region of the primer that is sufficiently complementary to the template to hybridize is referred to herein as the hybridizing region.

As used herein, a primer is “specific,” for a target sequence if, when used in an amplification reaction under sufficiently stringent conditions, the primer hybridizes primarily to the target nucleic acid. Typically, a primer is specific for a target sequence if the primer-target duplex stability is greater than the stability of a duplex formed between the primer and any other sequence found in the sample. One of skill in the art will recognize that various factors, such as salt conditions as well as base composition of the primer and the location of the mismatches, will affect the specificity of the primer, and that routine experimental confirmation of the primer specificity will be needed in many cases. Hybridization conditions can be chosen under which the primer can form stable duplexes only with a target sequence. Thus, the use of target-specific primers under suitably stringent amplification conditions enables the selective amplification of those target sequences that contain the target primer binding sites.

As used herein, a “polymerase” refers to an enzyme that catalyzes the polymerization of nucleotides. “DNA polymerase” catalyzes the polymerization of deoxyribonucleotides. Known DNA polymerases include, for example, Pyrococcus furiosus (Pfu) DNA polymerase, E. coli DNA polymerase I, T7 DNA polymerase and Thermus aquaticus (Taq) DNA polymerase, among others. “RNA polymerase” catalyzes the polymerization of ribonucleotides. The foregoing examples of DNA polymerases are also known as DNA-dependent DNA polymerases. RNA-dependent DNA polymerases also fall within the scope of DNA polymerases. Reverse transcriptase, which includes viral polymerases encoded by retroviruses, is an example of an RNA-dependent DNA polymerase. Known examples of RNA polymerase (“RNAP”) include, for example, T3 RNA polymerase, T7 RNA polymerase, SP6 RNA polymerase and E. coli RNA polymerase, among others. The foregoing examples of RNA polymerases are also known as DNA-dependent RNA polymerase. The polymerase activity of any of the above enzymes can be determined by means well known in the art.

The term “promoter” refers to a cis-acting DNA sequence that directs RNA polymerase and other trans-acting transcription factors to initiate RNA transcription from the DNA template that includes the cis-acting DNA sequence.

As used herein, the term “sequence defined biopolymer” refers to a biopolymer having a specific primary sequence. A sequence defined biopolymer can be equivalent to a genetically-encoded defined biopolymer in cases where a gene encodes the biopolymer having a specific primary sequence.

The polynucleotide sequences contemplated herein may be present in expression vectors. For example, the vectors may comprise: (a) a polynucleotide encoding an ORF of a protein; (b) a polynucleotide that expresses an RNA that directs RNA-mediated binding, nicking, and/or cleaving of a target DNA sequence; and both (a) and (b). The polynucleotide present in the vector may be operably linked to a prokaryotic or eukaryotic promoter. “Operably linked” refers to the situation in which a first nucleic acid sequence is placed in a functional relationship with a second nucleic acid sequence. For instance, a promoter is operably linked to a coding sequence if the promoter affects the transcription or expression of the coding sequence. Operably linked DNA sequences may be in close proximity or contiguous and, where necessary to join two protein coding regions, in the same reading frame. Vectors contemplated herein may comprise a heterologous promoter (e.g., a eukaryotic or prokaryotic promoter) operably linked to a polynucleotide that encodes a protein. A “heterologous promoter” refers to a promoter that is not the native or endogenous promoter for the protein or RNA that is being expressed. Vectors as disclosed herein may include plasmid vectors.

As used herein, “expression” refers to the process by which a polynucleotide is transcribed from a DNA template (such as into and mRNA or other RNA transcript) and/or the process by which a transcribed mRNA is subsequently translated into peptides, polypeptides, or proteins. Transcripts and encoded polypeptides may be collectively referred to as “gene product.” If the polynucleotide is derived from genomic DNA, expression may include splicing of the mRNA in a eukaryotic cell.

As used herein, “expression template” refers to a nucleic acid that serves as substrate for transcribing at least one RNA that can be translated into a sequence defined biopolymer (e.g., a polypeptide or protein). Expression templates include nucleic acids composed of DNA or RNA. Suitable sources of DNA for use a nucleic acid for an expression template include genomic DNA, cDNA and RNA that can be converted into cDNA. Genomic DNA, cDNA and RNA can be from any biological source, such as a tissue sample, a biopsy, a swab, sputum, a blood sample, a fecal sample, a urine sample, a scraping, among others. The genomic DNA, cDNA and RNA can be from host cell or virus origins and from any species, including extant and extinct organisms. As used herein, “expression template” and “transcription template” have the same meaning and are used interchangeably.

In certain exemplary embodiments, vectors such as, for example, expression vectors, containing a nucleic acid encoding one or more rRNAs or reporter polypeptides and/or proteins described herein are provided. As used herein, the term “vector” refers to a nucleic acid molecule capable of transporting another nucleic acid to which it has been linked. One type of vector is a “plasmid,” which refers to a circular double stranded DNA loop into which additional DNA segments can be ligated. Such vectors are referred to herein as “expression vectors.” In general, expression vectors of utility in recombinant DNA techniques are often in the form of plasmids. In the present specification, “plasmid” and “vector” can be used interchangeably. However, the disclosed methods and compositions are intended to include such other forms of expression vectors, such as viral vectors (e.g., replication defective retroviruses, adenoviruses and adeno-associated viruses), which serve equivalent functions.

In certain exemplary embodiments, the recombinant expression vectors comprise a nucleic acid sequence (e.g., a nucleic acid sequence encoding one or more rRNAs or reporter polypeptides and/or proteins described herein) in a form suitable for expression of the nucleic acid sequence in one or more of the methods described herein, which means that the recombinant expression vectors include one or more regulatory sequences which is operatively linked to the nucleic acid sequence to be expressed. Within a recombinant expression vector, “operably linked” is intended to mean that the nucleotide sequence encoding one or more rRNAs or reporter polypeptides and/or proteins described herein is linked to the regulatory sequence(s) in a manner which allows for expression of the nucleotide sequence (e.g., in an in vitro transcription and/or translation system). The term “regulatory sequence” is intended to include promoters, enhancers and other expression control elements (e.g., polyadenylation signals). Such regulatory sequences are described, for example, in Goeddel; Gene Expression Technology: Methods in Enzymology 185, Academic Press, San Diego, Calif (1990).

Oligonucleotides and polynucleotides may optionally include one or more non-standard nucleotide(s), nucleotide analog(s) and/or modified nucleotides. Examples of modified nucleotides include, but are not limited to diaminopurine, S²T, 5-fluorouracil, 5-bromouracil, 5-chlorouracil, 5-iodouracil, hypoxanthine, xantine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-D46-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, 2,6-diaminopurine and the like. Nucleic acid molecules may also be modified at the base moiety (e.g., at one or more atoms that typically are available to form a hydrogen bond with a complementary nucleotide and/or at one or more atoms that are not typically capable of forming a hydrogen bond with a complementary nucleotide), sugar moiety or phosphate backbone.

The terms “polynucleotide,” “polynucleotide sequence,” “nucleic acid” and “nucleic acid sequence” refer to a nucleotide, oligonucleotide, polynucleotide (which terms may be used interchangeably), or any fragment thereof. These phrases also refer to DNA or RNA of genomic, natural, or synthetic origin (which may be single-stranded or double-stranded and may represent the sense or the antisense strand).

Regarding polynucleotide sequences, the terms “percent identity” and “% identity” refer to the percentage of residue matches between at least two polynucleotide sequences aligned using a standardized algorithm. Such an algorithm may insert, in a standardized and reproducible way, gaps in the sequences being compared in order to optimize alignment between two sequences, and therefore achieve a more meaningful comparison of the two sequences. Percent identity for a nucleic acid sequence may be determined as understood in the art. (See, e.g., U.S. Pat. No. 7,396,664, which is incorporated herein by reference in its entirety). A suite of commonly used and freely available sequence comparison algorithms is provided by the National Center for Biotechnology Information (NCBI) Basic Local Alignment Search Tool (BLAST), which is available from several sources, including the NCBI, Bethesda, Md., at its website. The BLAST software suite includes various sequence analysis programs including “blastn,” that is used to align a known polynucleotide sequence with other polynucleotide sequences from a variety of databases. Also available is a tool called “BLAST 2 Sequences” that is used for direct pairwise comparison of two nucleotide sequences. “BLAST 2 Sequences” can be accessed and used interactively at the NCBI website. The “BLAST 2 Sequences” tool can be used for both blastn and blastp (discussed above).

Regarding polynucleotide sequences, percent identity may be measured over the length of an entire defined polynucleotide sequence, for example, as defined by a particular SEQ ID number, or may be measured over a shorter length, for example, over the length of a fragment taken from a larger, defined sequence, for instance, a fragment of at least 20, at least 30, at least 40, at least 50, at least 70, at least 100, or at least 200 contiguous nucleotides. Such lengths are exemplary only, and it is understood that any fragment length supported by the sequences shown herein, in the tables, figures, or Sequence Listing, may be used to describe a length over which percentage identity may be measured.

Regarding polynucleotide sequences, “variant,” “mutant,” or “derivative” may be defined as a nucleic acid sequence having at least 50% sequence identity to the particular nucleic acid sequence over a certain length of one of the nucleic acid sequences using blastn with the “BLAST 2 Sequences” tool available at the National Center for Biotechnology Information's website. (See Tatiana A. Tatusova, Thomas L. Madden (1999), “Blast 2 sequences—a new tool for comparing protein and nucleotide sequences”, FEMS Microbiol Lett. 174:247-250). Such a pair of nucleic acids may show, for example, at least 60%, at least 70%, at least 80%, at least 85%, at least 90%, at least 91%, at least 92%, at least 93%, at least 94%, at least 95%, at least 96%, at least 97%, at least 98%, or at least 99% or greater sequence identity over a certain defined length.

Nucleic acid sequences that do not show a high degree of identity may nevertheless encode similar amino acid sequences due to the degeneracy of the genetic code where multiple codons may encode for a single amino acid. It is understood that changes in a nucleic acid sequence can be made using this degeneracy to produce multiple nucleic acid sequences that all encode substantially the same protein. For example, polynucleotide sequences as contemplated herein may encode a protein and may be codon-optimized for expression in a particular host. In the art, codon usage frequency tables have been prepared for a number of host organisms including humans, mouse, rat, pig, E. coli, plants, and other host cells.

A “recombinant nucleic acid” is a sequence that is not naturally occurring or has a sequence that is made by an artificial combination of two or more otherwise separated segments of sequence. This artificial combination is often accomplished by chemical synthesis or, more commonly, by the artificial manipulation of isolated segments of nucleic acids, e.g., by genetic engineering techniques known in the art. The term recombinant includes nucleic acids that have been altered solely by addition, substitution, or deletion of a portion of the nucleic acid. Frequently, a recombinant nucleic acid may include a nucleic acid sequence operably linked to a promoter sequence. Such a recombinant nucleic acid may be part of a vector that is used, for example, to transform a cell.

The nucleic acids disclosed herein may be “substantially isolated or purified.” The term “substantially isolated or purified” refers to a nucleic acid that is removed from its natural environment, and is at least 60% free, preferably at least 75% free, and more preferably at least 90% free, even more preferably at least 95% free from other components with which it is naturally associated.

### Peptides, Polypeptides, Proteins, and Synthesis Methods

As used herein, the terms “peptide,” “polypeptide,” and “protein,” refer to molecules comprising a chain a polymer of amino acid residues joined by amide linkages. The term “amino acid residue,” includes but is not limited to amino acid residues contained in the group consisting of alanine (Ala or A), cysteine (Cys or C), aspartic acid (Asp or D), glutamic acid (Glu or E), phenylalanine (Phe or F), glycine (Gly or G), histidine (His or H), isoleucine (Ile or I), lysine (Lys or K), leucine (Leu or L), methionine (Met or M), asparagine (Asn or N), proline (Pro or P), glutamine (Gln or Q), arginine (Arg or R), serine (Ser or S), threonine (Thr or T), valine (Val or V), tryptophan (Trp or W), and tyrosine (Tyr or Y) residues. The term “amino acid residue” also may include nonstandard or unnatural amino acids. The term “amino acid residue” may include alpha-, beta-, gamma-, and delta-amino acids.

In some embodiments, the term “amino acid residue” may include nonstandard or unnatural amino acid residues contained in the group consisting of homocysteine, 2-Aminoadipic acid, N-Ethylasparagine, 3-Aminoadipic acid, Hydroxylysine, β-alanine, β-Amino-propionic acid, allo-Hydroxylysine acid, 2-Aminobutyric acid, 3-Hydroxyproline, 4-Aminobutyric acid, 4-Hydroxyproline, piperidinic acid, 6-Aminocaproic acid, Isodesmosine, 2-Aminoheptanoic acid, allo-Isoleucine, 2-Aminoisobutyric acid, N-Methylglycine, sarcosine, 3-Aminoisobutyric acid, N-Methylisoleucine, 2-Aminopimelic acid, 6-N-Methyllysine, 2,4-Diaminobutyric acid, N-Methylvaline, Desmosine, Norvaline, 2,2′-Diaminopimelic acid, Norleucine, 2,3-Diaminopropionic acid, Ornithine, and N-Ethylglycine. The term “amino acid residue” may include L isomers or D isomers of any of the aforementioned amino acids.

Other examples of nonstandard or unnatural amino acids include, but are not limited, to a p-acetyl-L-phenylalanine, a p-iodo-L-phenylalanine, an O-methyl-L-tyrosine, a p-propargyloxyphenylalanine, a p-propargyl-phenylalanine, an L-3-(2-naphthyl)alanine, a 3-methyl-phenylalanine, an O-4-allyl-L-tyrosine, a 4-propyl-L-tyrosine, a tri-O-acetyl-GlcNAcpp-serine, an L-Dopa, a fluorinated phenylalanine, an isopropyl-L-phenylalanine, a p-azido-L-phenylalanine, a p-acyl-L-phenylalanine, a p-benzoyl-L-phenylalanine, an L-phosphoserine, a phosphonoserine, a phosphonotyrosine, a p-bromophenylalanine, a p-amino-L-phenylalanine, an isopropyl-L-phenylalanine, an unnatural analogue of a tyrosine amino acid; an unnatural analogue of a glutamine amino acid; an unnatural analogue of a phenylalanine amino acid; an unnatural analogue of a serine amino acid; an unnatural analogue of a threonine amino acid; an unnatural analogue of a methionine amino acid; an unnatural analogue of a leucine amino acid; an unnatural analogue of a isoleucine amino acid; an alkyl, aryl, acyl, azido, cyano, halo, hydrazine, hydrazide, hydroxyl, alkenyl, alkynl, ether, thiol, sulfonyl, seleno, ester, thioacid, borate, boronate, 26ufa26hor, phosphono, phosphine, heterocyclic, enone, imine, aldehyde, hydroxylamine, keto, or amino substituted amino acid, or a combination thereof, an amino acid with a photoactivatable cross-linker; a spin-labeled amino acid; a fluorescent amino acid; a metal binding amino acid; a metal-containing amino acid; a radioactive amino acid; a photocaged and/or photoisomerizable amino acid; a biotin or biotin-analogue containing amino acid; a keto containing amino acid; an amino acid comprising polyethylene glycol or polyether; a heavy atom substituted amino acid; a chemically cleavable or photocleavable amino acid; an amino acid with an elongated side chain; an amino acid containing a toxic group; a sugar substituted amino acid; a carbon-linked sugar-containing amino acid; a redox-active amino acid; an α-hydroxy containing acid; an amino thio acid; an α,α disubstituted amino acid; a β-amino acid; a γ-amino acid, a cyclic amino acid other than proline or histidine, and an aromatic amino acid other than phenylalanine, tyrosine or tryptophan.

As used herein, a “peptide” is defined as a short polymer of amino acids, of a length typically of 20 or less amino acids, and more typically of a length of 12 or less amino acids (Garrett & Grisham, Biochemistry, 2^{nd} edition, 1999, Brooks/Cole, 110). In some embodiments, a peptide as contemplated herein may include no more than about 2, 3, 4, 5, 6, 7, 8, 9, 10, 11, 12, 13, 14, 15, 16, 17, 18, 19, or 20 amino acids. A polypeptide, also referred to as a protein, is typically of length ≥100 amino acids (Garrett & Grisham, Biochemistry, 2^{nd} edition, 1999, Brooks/Cole, 110). A polypeptide, as contemplated herein, may comprise, but is not limited to, 100, 101, 102, 103, 104, 105, about 110, about 120, about 130, about 140, about 150, about 160, about 170, about 180, about 190, about 200, about 210, about 220, about 230, about 240, about 250, about 275, about 300, about 325, about 350, about 375, about 400, about 425, about 450, about 475, about 500, about 525, about 550, about 575, about 600, about 625, about 650, about 675, about 700, about 725, about 750, about 775, about 800, about 825, about 850, about 875, about 900, about 925, about 950, about 975, about 1000, about 1100, about 1200, about 1300, about 1400, about 1500, about 1750, about 2000, about 2250, about 2500 or more amino acid residues.

A peptide as contemplated herein may be further modified to include non-amino acid moieties. Modifications may include but are not limited to acylation (e.g., O-acylation (esters), N-acylation (amides), S-acylation (thioesters)), acetylation (e.g., the addition of an acetyl group, either at the N-terminus of the protein or at lysine residues), formylation lipoylation (e.g., attachment of a lipoate, a C8 functional group), myristoylation (e.g., attachment of myristate, a C14 saturated acid), palmitoylation (e.g., attachment of palmitate, a C16 saturated acid), alkylation (e.g., the addition of an alkyl group, such as an methyl at a lysine or arginine residue), isoprenylation or prenylation (e.g., the addition of an isoprenoid group such as farnesol or geranylgeraniol), amidation at C-terminus, glycosylation (e.g., the addition of a glycosyl group to either asparagine, hydroxylysine, serine, or threonine, resulting in a glycoprotein). Distinct from glycation, which is regarded as a nonenzymatic attachment of sugars, polysialylation (e.g., the addition of polysialic acid), glypiation (e.g., glycosylphosphatidylinositol (GPI) anchor formation, hydroxylation, iodination (e.g., of thyroid hormones), and phosphorylation (e.g., the addition of a phosphate group, usually to serine, tyrosine, threonine or histidine).

The modified amino acid sequences that are disclosed herein may include a deletion in one or more amino acids. As utilized herein, a “deletion” means the removal of one or more amino acids relative to the native amino acid sequence. The modified amino acid sequences that are disclosed herein may include an insertion of one or more amino acids. As utilized herein, an “insertion” means the addition of one or more amino acids to a native amino acid sequence. The modified amino acid sequences that are disclosed herein may include a substitution of one or more amino acids. As utilized herein, a “substitution” means replacement of an amino acid of a native amino acid sequence with an amino acid that is not native to the amino acid sequence.

For example, the modified NGTs disclosed herein may include one or more deletions, insertions, and/or substitutions in order modified the native amino acid sequence of the enzyme to enhance function.

As another example, an “acceptor peptide” or “acceptor peptide sequence” is modified to include one or more heterologous amino acid motifs that are glycosylated by an N-glycosyltransferase. As used herein, the term “acceptor peptide” or “acceptor peptide sequence” refers to the peptide sequence that is targeted by NGTs for glycosylation. An exemplary acceptor peptide sequence is [X₋₁]-[N]-[X₊₁]-[S/T], where X is any canonical amino acid, optionally where [X₊₁] is not P. Another example of an acceptor peptide sequence is [X₋₁]-[N]-[X₊₁]-[X₊₂], where X is any canonical amino acid, optionally where [X₊₁] is not P, and optionally where [X₊₂] is not S or T. Another example of an acceptor peptide sequence is [X₋₂]-[X₋₁]-[N]-[X₊₁]-[X₊₂]-[X₊₃], where X is any canonical amino acid, optionally where [X₊₁] is not P, and optionally where [X₊₂] is not S or T As used herein the term “target polypeptide” refers to a polypeptide that may be modified, purified, isolated or further studied. In some embodiments, a target polypeptide comprises an acceptor peptide sequence and is glycosylated by an NGT.

Regarding proteins, a “deletion” refers to a change in the amino acid sequence that results in the absence of one or more amino acid residues. A deletion may remove at least 1, 2, 3, 4, 5, 10, 20, 50, 100, 200, or more amino acids residues. A deletion may include an internal deletion and/or a terminal deletion (e.g., an N-terminal truncation, a C-terminal truncation or both of a reference polypeptide). A “variant,” “mutant,” or “derivative” of a reference polypeptide sequence may include a deletion relative to the reference polypeptide sequence.

Regarding proteins, “fragment” is a portion of an amino acid sequence which is identical in sequence to but shorter in length than a reference sequence. A fragment may comprise up to the entire length of the reference sequence, minus at least one amino acid residue. For example, a fragment may comprise from 5 to 1000 contiguous amino acid residues of a reference polypeptide, respectively. In some embodiments, a fragment may comprise at least 5, 10, 15, 20, 25, 30, 40, 50, 60, 70, 80, 90, 100, 150, 250, or 500 contiguous amino acid residues of a reference polypeptide. Fragments may be preferentially selected from certain regions of a molecule. The term “at least a fragment” encompasses the full-length polypeptide. A fragment may include an N-terminal truncation, a C-terminal truncation, or both truncations relative to the full-length protein. A “variant,” “mutant,” or “derivative” of a reference polypeptide sequence may include a fragment of the reference polypeptide sequence.

Regarding proteins, the words “insertion” and “addition” refer to changes in an amino acid sequence resulting in the addition of one or more amino acid residues. An insertion or addition may refer to 1, 2, 3, 4, 5, 10, 20, 30, 40, 50, 60, 70, 80, 90, 100, 150, 200, or more amino acid residues. A “variant,” “mutant,” or “derivative” of a reference polypeptide sequence may include an insertion or addition relative to the reference polypeptide sequence. A variant of a protein may have N-terminal insertions, C-terminal insertions, internal insertions, or any combination of N-terminal insertions, C-terminal insertions, and internal insertions.

Regarding proteins, the phrases “percent identity” and “% identity,” refer to the percentage of residue matches between at least two amino acid sequences aligned using a standardized algorithm. Methods of amino acid sequence alignment are well-known. Some alignment methods take into account conservative amino acid substitutions. Such conservative substitutions, explained in more detail below, generally preserve the charge and hydrophobicity at the site of substitution, thus preserving the structure (and therefore function) of the polypeptide. Percent identity for amino acid sequences may be determined as understood in the art. (See, e.g., U.S. Pat. No. 7,396,664, which is incorporated herein by reference in its entirety). A suite of commonly used and freely available sequence comparison algorithms is provided by the National Center for Biotechnology Information (NCBI) Basic Local Alignment Search Tool (BLAST), which is available from several sources, including the NCBI, Bethesda, Md., at its website. The BLAST software suite includes various sequence analysis programs including “blastp,” that is used to align a known amino acid sequence with other amino acids sequences from a variety of databases.

Regarding proteins, percent identity may be measured over the length of an entire defined polypeptide sequence, for example, as defined by a particular SEQ ID number, or may be measured over a shorter length, for example, over the length of a fragment taken from a larger, defined polypeptide sequence, for instance, a fragment of at least 15, at least 20, at least 30, at least 40, at least 50, at least 70 or at least 150 contiguous residues. Such lengths are exemplary only, and it is understood that any fragment length supported by the sequences shown herein, in the tables, figures or Sequence Listing, may be used to describe a length over which percentage identity may be measured.

Regarding proteins, the amino acid sequences of variants, mutants, or derivatives as contemplated herein may include conservative amino acid substitutions relative to a reference amino acid sequence. For example, a variant, mutant, or derivative protein may include conservative amino acid substitutions relative to a reference molecule. “Conservative amino acid substitutions” are those substitutions that are a substitution of an amino acid for a different amino acid where the substitution is predicted to interfere least with the properties of the reference polypeptide. In other words, conservative amino acid substitutions substantially conserve the structure and the function of the reference polypeptide. The following table provides a list of exemplary conservative amino acid substitutions which are contemplated herein:

| Original | |
|---|---|
| Residue | Conservative Substitution |
| Ala | Gly, Ser |
| Arg | His, Lys |
| Asn | Asp, Gln, His |
| Asp | Asn, Glu |
| Cys | Ala, Ser |
| Gln | Asn, Glu, His |
| Glu | Asp, Gln, His |
| Gly | Ala |
| His | Asn, Arg, Gln, Glu |
| Ile | Leu, Val |
| Leu | Ile, Val |
| Lys | Arg, Gln, Glu |
| Met | Leu, Ile |
| Phe | His, Met, Leu, Trp, Tyr |
| Ser | Cys, Thr |
| Thr | Ser, Val |
| Trp | Phe, Tyr |
| Tyr | His, Phe, Trp |
| Val | Ile, Leu, Thr |

Conservative amino acid substitutions generally maintain (a) the structure of the polypeptide backbone in the area of the substitution, for example, as a beta sheet or alpha helical conformation, (b) the charge or hydrophobicity of the molecule at the site of the substitution, and/or (c) the bulk of the side chain. Non-conservative amino acids typically disrupt (a) the structure of the polypeptide backbone in the area of the substitution, for example, as a beta sheet or alpha helical conformation, (b) the charge or hydrophobicity of the molecule at the site of the substitution, and/or (c) the bulk of the side chain.

The disclosed proteins, mutants, or variants, described herein may have one or more functional or biological activities exhibited by a reference polypeptide (e.g., one or more functional or biological activities exhibited by wild-type protein). In some embodiments, the activity of the variant or mutant protein (e.g., a modified NGT as disclosed herein) may have an activity that is enhanced, as compared to a comparable wild-type or control NGT enzyme, or may have an alternative or a modified activity as compared to a comparable or wild-type or control NGT enzyme. By way of example, but not by way of limitation, the modified NGTs' disclosed herein have the ability to glycosylate unique or non-canonical target peptide sequences, and/or have increased glycosylation efficiency as compared to a wild-type or control NGT. An exemplary wild-type NGT is the NGT of Actinobacillus pleuropneumoniae the amino acid sequence of which is provided as SEQ ID NO:1.

```
Actinobacillus pleuropneumonieae NGT
(SEQ ID NO: 1)
    1 menenkpnva nfeaavaakd yekacselll ilsqldsnfg giheiefeyp aqlqdlegek
  61 ivyfctrmat aittlfsdpv leisdlgvqr flvyqrwlal ifasspfvna dhilqtynre
121 pnrknsleih ldssksslik fcilylpesn vnlnldvmwn ispelcaslc falqsprfvg
181 tstafnkrat ilqwfprhld qlknlnnips aishdvymhc sydtsvnkhd vkralnhvir
241 rhieseygwk drdvahigyr nnkpvmvvll ehfhsahsiy rthstsmiaa rehfyliglg
301 spsvdqagqe vfdefhlvag dnmkqklefi rsvcesngaa ifympsigmd mttifasntr
361 lapiqaialg hpatthsdfi eyviveddyv gseecfsetl lrlpkdalpy vpsalapekv
421 dyllrenpev vnigiasttm klnpyfleal kairdrakvk vhfhfalgqs ngithpyver
481 fiksylgdsa tahphspyhq ylrilhncdm mvnpfpfgnt ngiidmvtlg lvgvcktgae
541 vhehideglf krlglpewli antvdeyver avrlaenhqe rlelrryiie nnglntlftg
601 dprpmgqvfl eklnaflken
Actinobacillus pleuropneumonieae NGT
(SEQ ID NO: 1a)
    1 menenkpnva nfeaavaakd yekacselll ilsqldsnfg giheiefeyp aqlqdleqek
  61 ivyfctrmat aittlfsdpv leisdlgvqr flvyqrwlal ifasspfvna dhilqtynre
121 pnrknsleih ldssksslik fcilylpesn vnlnldvmwn ispelcaslc falqsprfvg
181 tstafnkrat ilqwfprhld qlknlnnips aishdvymhc sydtsvnkhd vkralnhvir
241 rhieseygwk drdvahigyr nnkpvmvvll ehfhsahsiy rthstsmiaa rehfyliglg
301 spsvdqagqe vfdefhlvag dnmkqklefi rsvcesngaa ifympsigmd mttifasntr
361 lapiqaialg hpatthsdfi eyviveddyv gseecfsetl lrlpkdalpy vpsalapekv
421 dyllrenpev vnigiasttm klnpyfleal kairdrakvk vhfhfalgas ngithpyver
481 fiksylgdsa tahphspyhq ylrilhncdm mvnpfpfgnt ngiidmvtlg lvgvcktgae
541 vhehideglf krlglpewli antvdeyver avrlaenhqe rlelrryiie nnglntlftg
601 dprpmgqvfl eklnaflken
```

### Modified NGT

Amino acid SEQ ID NO: 1a (shown above) is identical to SEQ ID NO:1, except for the single amino acid substitution of Q469A. In some embodiments, modified NGT's are described with reference to SEQ ID NO: 1a, and changes at amino acid position 469 are referred to as “A469X”. As is known in the art, sources of NGT can be derived from a variety of bacteria. By way of example, but not by way of limitation, exemplary bacteria include Actinobacillus spp., Escherichia spp., Haemophilus spp., or Mannheimia spp. In some embodiments, an NGT is derived from Actinobacillus pleuropneumoniae, Haemophilus influenza, Mannheimia haemolytica, Haemophilus dureyi, Yersinia pestis, or Escherichia coli. Disclosed herein are modified N-glycosyltransferases (NGTs), method for generating modified NGTs, and methods for preparing glycoproteins and recombinant glycoproteins in vitro and in vivo using the modified NGTs. In some embodiments, the NGTs disclosed herein include one or more substitution mutations, and typically glycosylate a wider array of acceptor peptide sequences as compared to an unmodified NGT under the same reaction conditions, and/or have an affinity for a wider array of acceptor peptide sequences as compared to an unmodified NGT under the same reaction conditions.

In some embodiments, the NGTs of the present disclosure include a peptide binding pocket. In some embodiments, the peptide binding pocket comprises amino acids F39, R177, H214, D215, M218, H219, Y222, H272, H277, S278, 1279, R281, M349, G370, H371, T438, T439, M440, K441, A469, H495, P497, Y498, F517, N521, D525, for example, of Actinobacillus pleuropneumoniae NGT or equivalent amino acid positions in another NGT of a different organism. In some embodiments, the one or more substitution mutations is in a binding pocket amino acid. By way of example, but not by way of limitation, in some embodiments, the modified NGT comprises one or more mutations at amino acid position H219, T438, A496 (or Q496 for ApNGT) or H495 of e.g., SEQ ID NO:1 (or 1a), or equivalent positions in another NGT. In some embodiments, a NGT comprising SEQ ID NO:1 or 1a, includes at least one substitution mutation selected from the group consisting of: H219F, H219W, T438S, T439E, A469G, A469I, H495D, H219F-T438S, H219F-H495D, H219W-T438S, H219W-H495D, A469G-H495D, and A469I-H495D.

The disclosed proteins may be substantially isolated or purified. The term “substantially isolated or purified” refers to proteins that are removed from their natural environment, and are at least 60% free, preferably at least 75% free, and more preferably at least 90% free, even more preferably at least 95% free from other components with which they are naturally associated.

As is known in the art, NGTs from different organisms may have differences in amino acid sequence. Thus, a mutation at amino acid position 469 of an Actinobacillus species NGT, such as Actinobacillus pleuropneumoniae (ApNGT), may have “an equivalent,” although not exact, counterpart of amino acid position 469 in an NGT of another species. Thus, as used herein the term “an equivalent” when referring to a mutant amino acid position, means the comparable position in the amino acid sequence of another NGT.

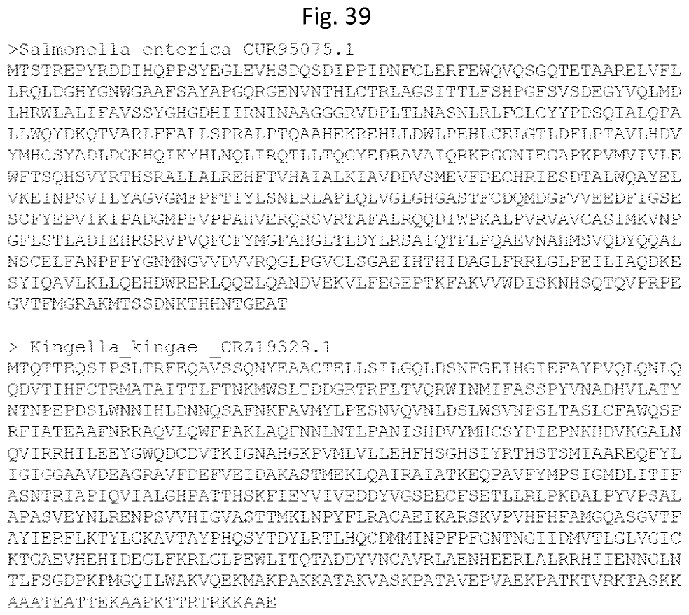

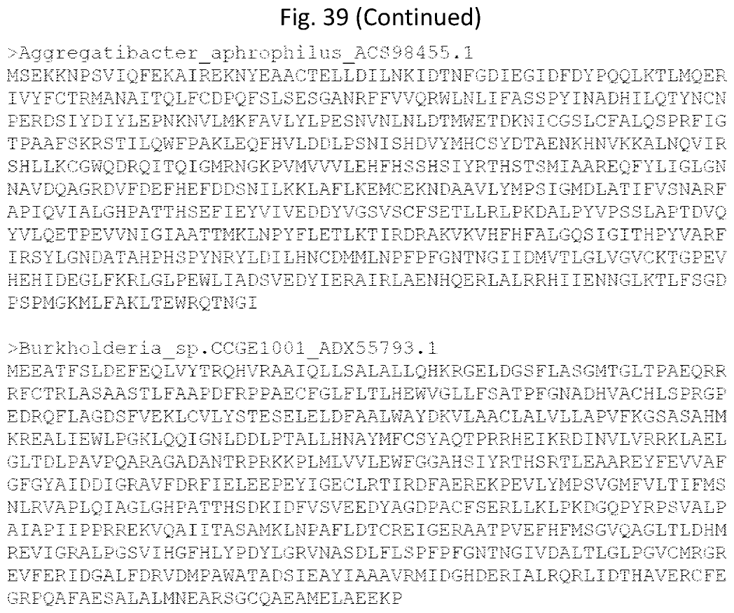

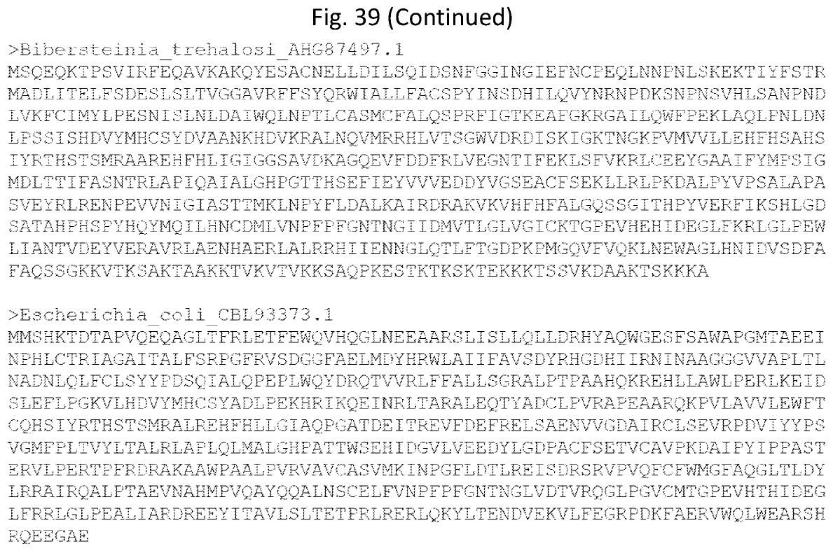

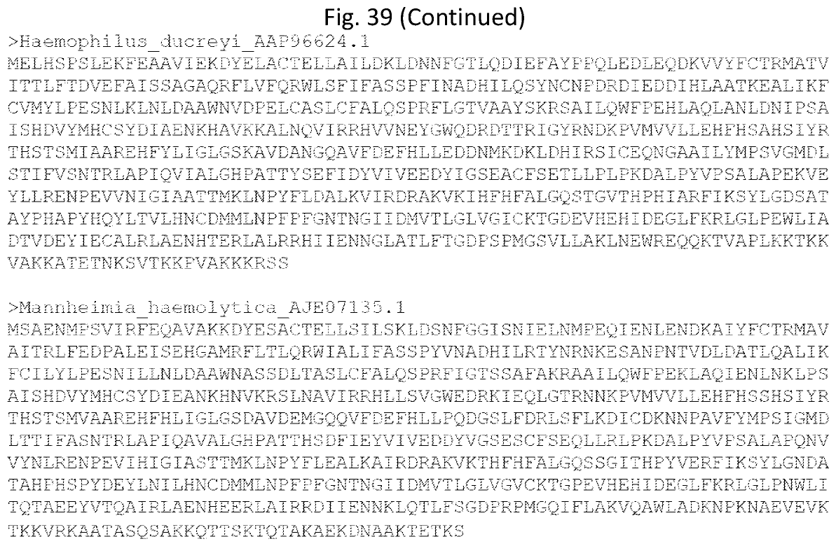

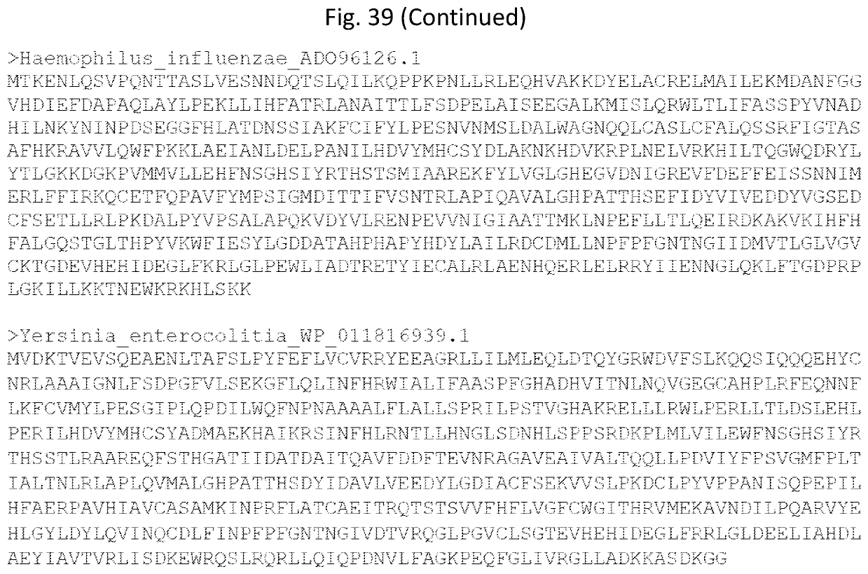

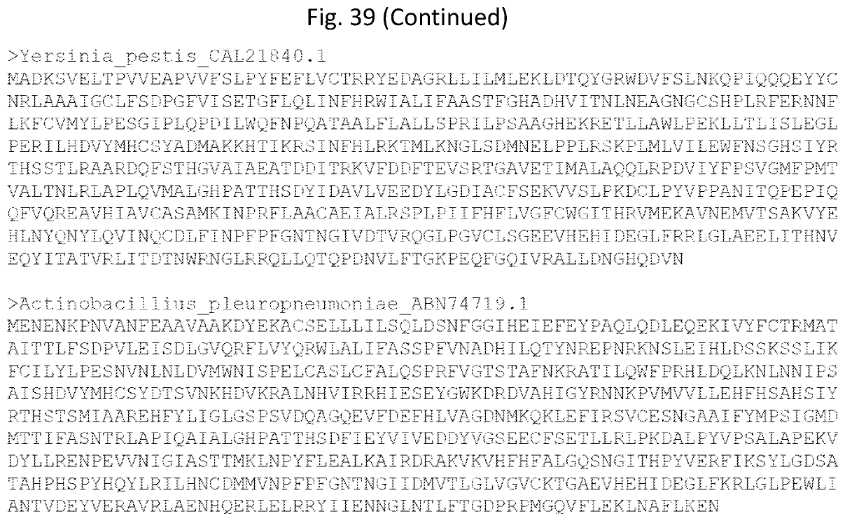

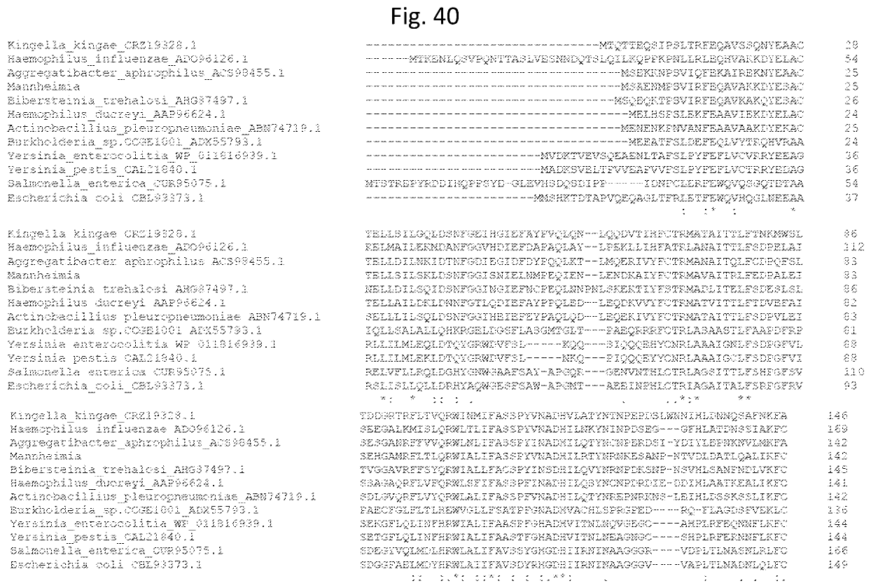

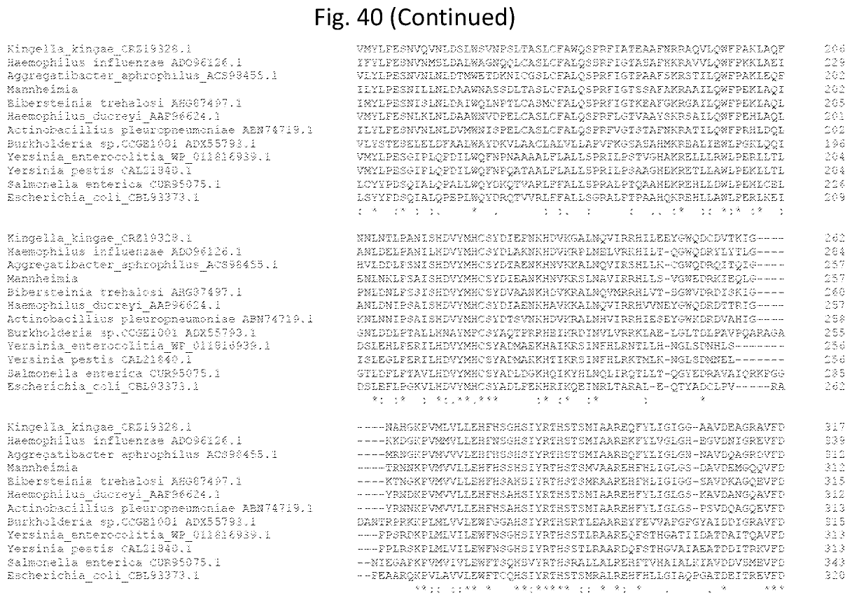

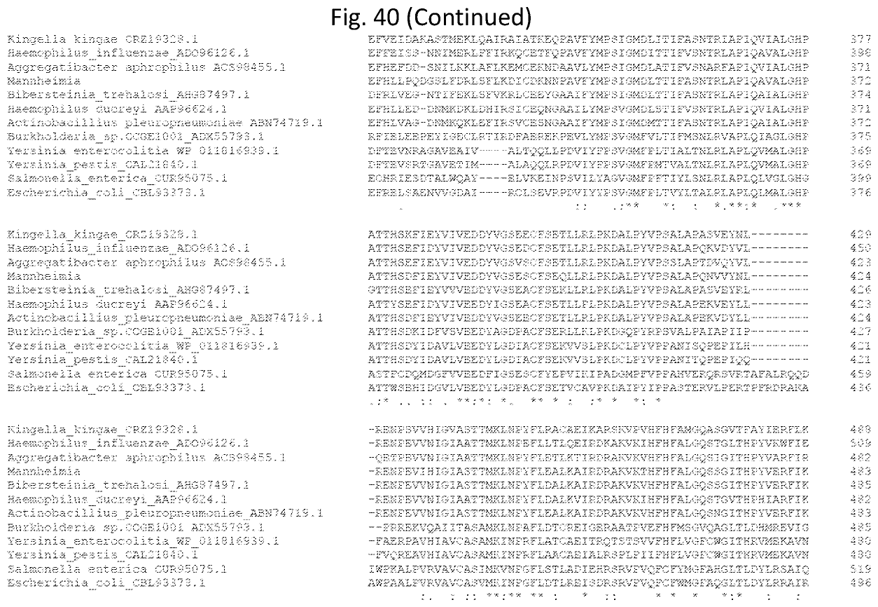

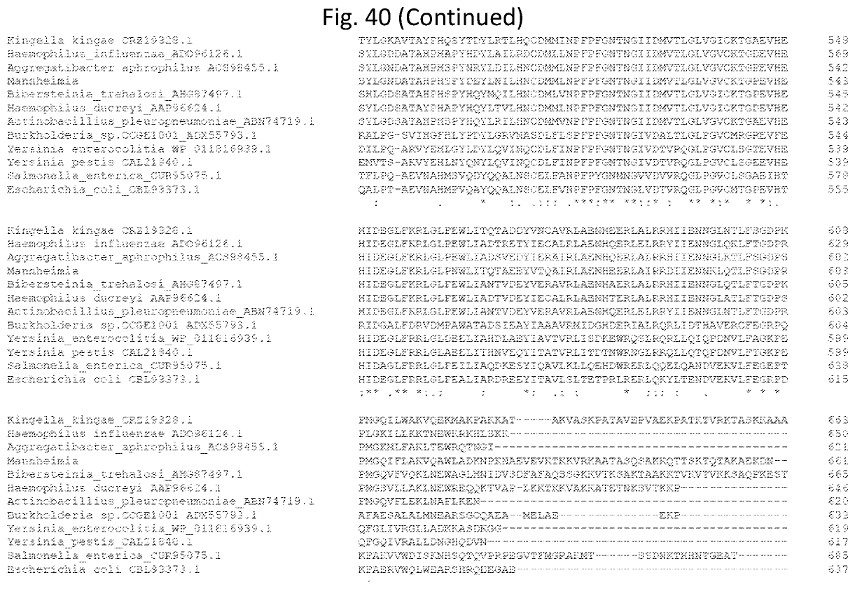

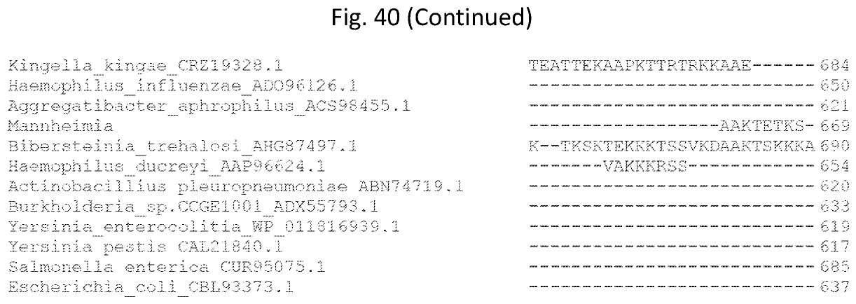

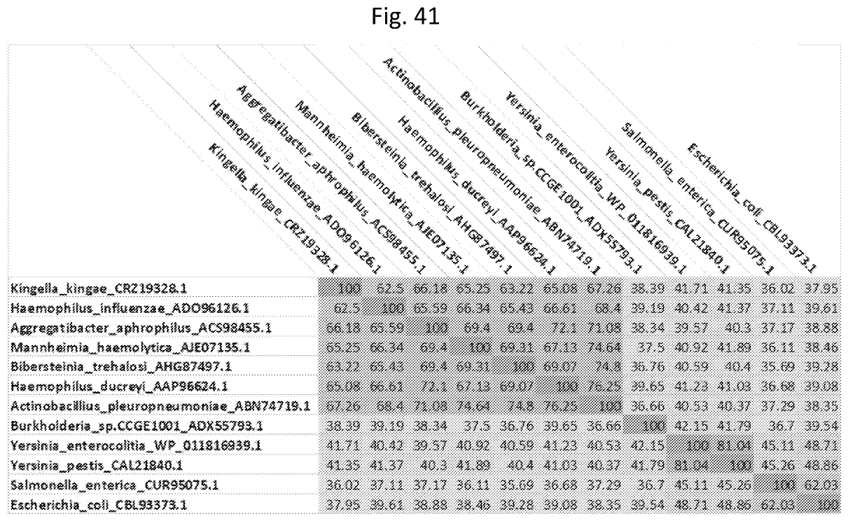

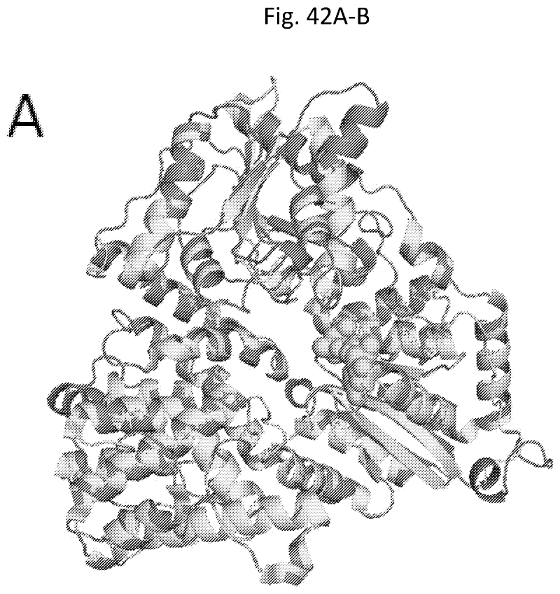

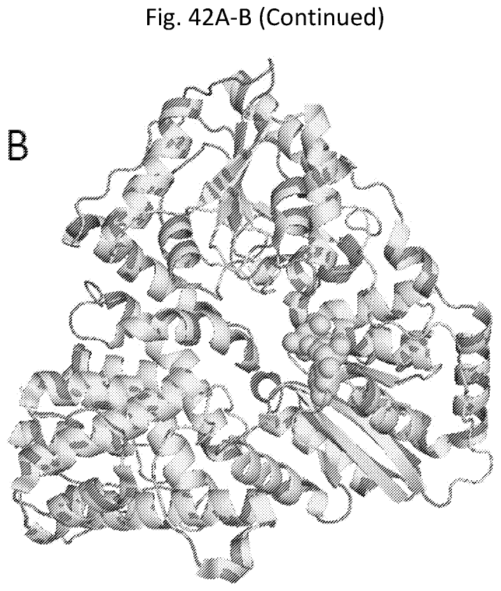

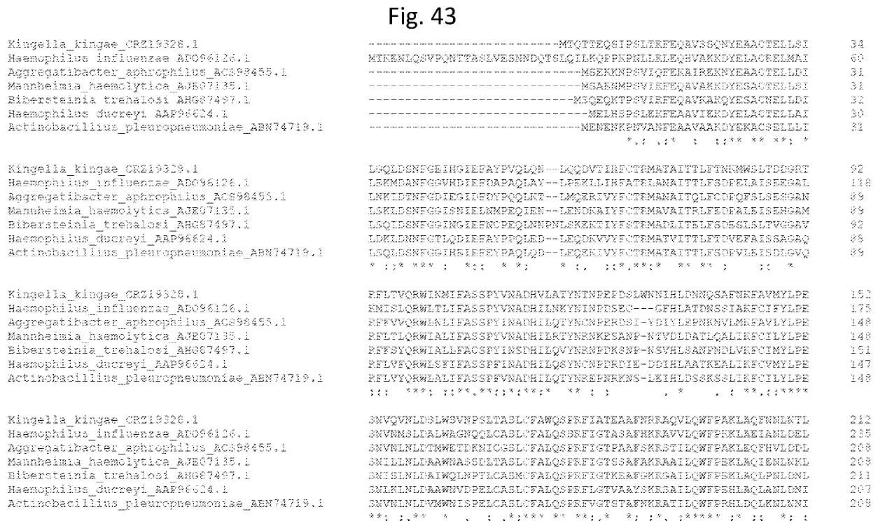

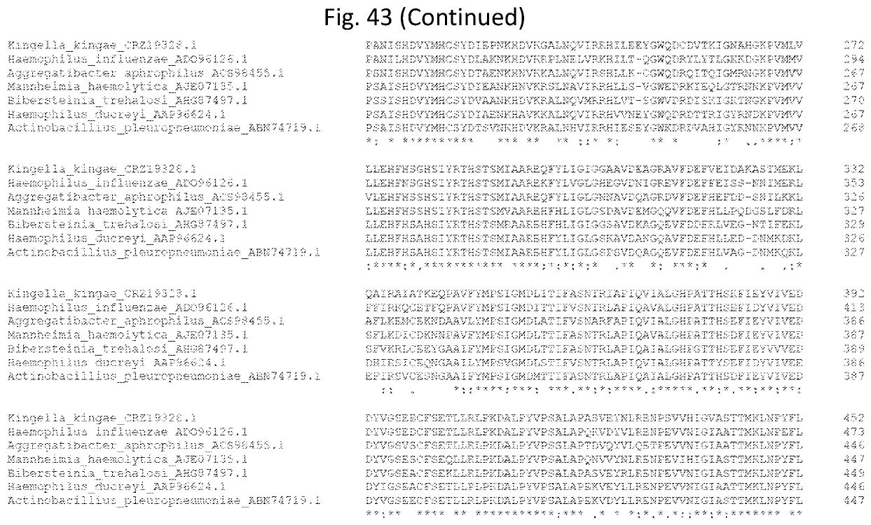

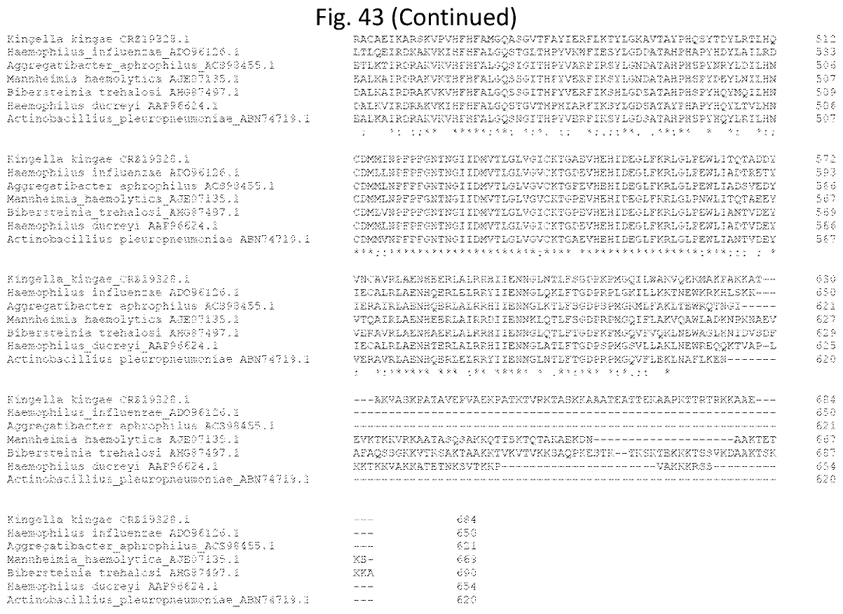

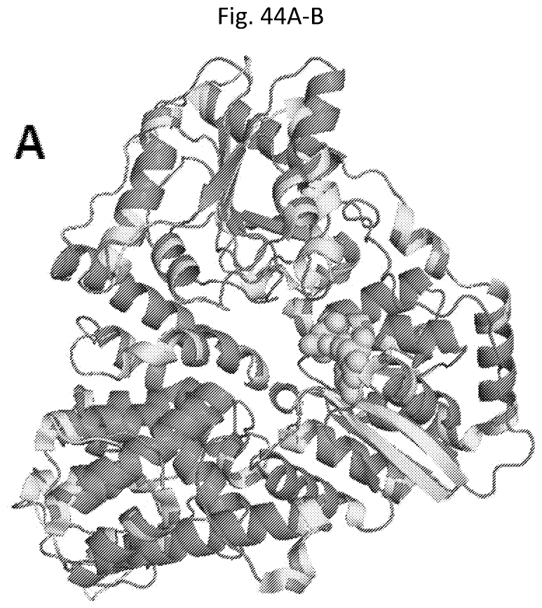

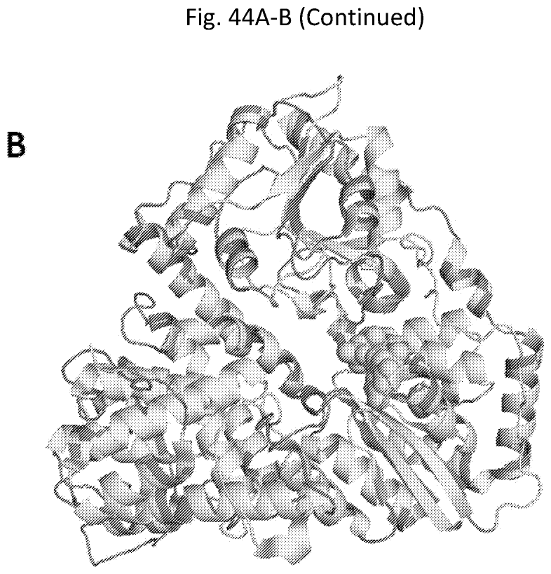

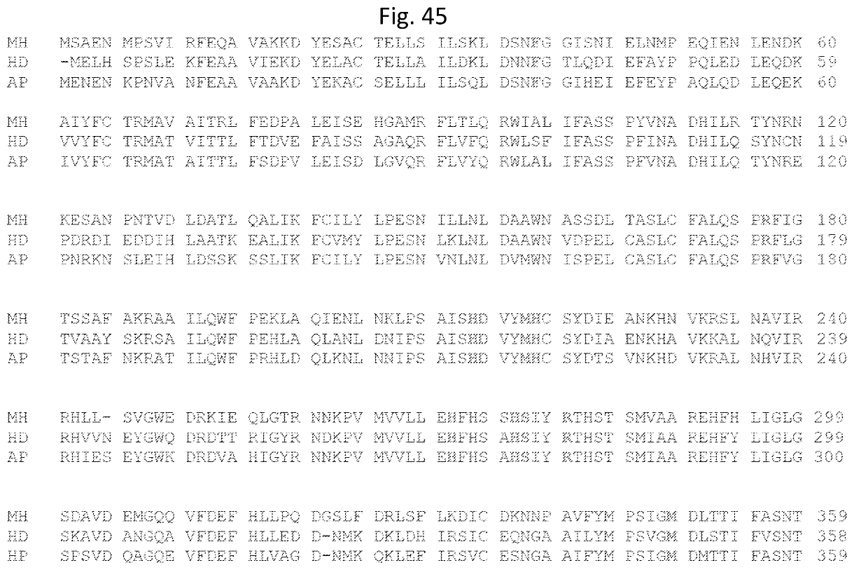

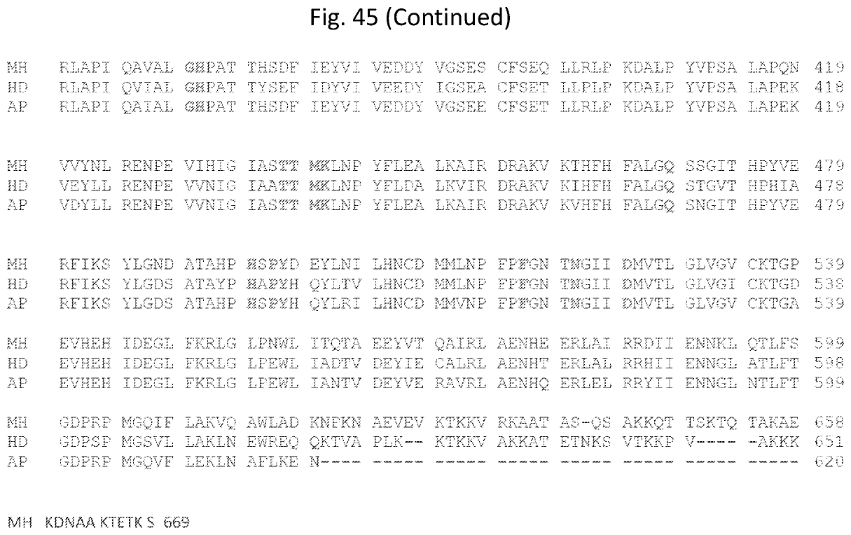

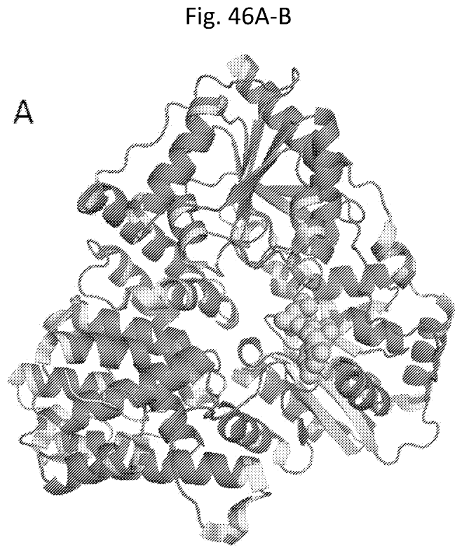

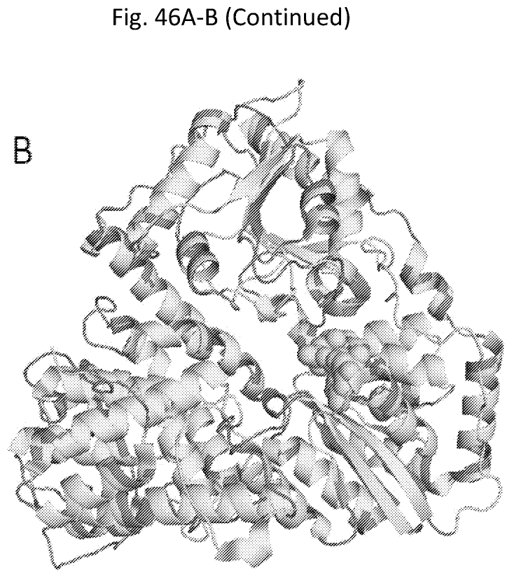

While the NGT from Actinobacillus pleuropneumoniae is exemplified herein in detail, it is understood that NGTs from other organisms can be modified in similar fashion, and result in a modified NGT with the same or similar altered function (e.g., glycosylate a wider array of acceptor peptide sequences as compared to the unmodified NGT control under the same reaction conditions, and/or have an affinity for a wider array of acceptor peptide sequences as compared to the unmodified NGT control the same reaction conditions). To this end, in addition to the amino acid sequence of Actinobacillius pleuropneumoniae NGT (SEQ ID NO: 1), the amino acid sequences of 10 different NGTs from 10 different organisms are provided in FIG. 39. FIGS. 40, 43, and 45 provide alignments of these sequences, and FIG. 41 provides a summary of table showing the percent identity among these NGTs. FIGS. 42, 44, and 46 show the structural alignments between several of these NGTs, illustrating the correspondence of structural and functional domains, such as the binding pocket. For example, FIG. 45 shows an alignment of Mannheimia haemolytica (MH), Haemophilus ducreyi (HD), and Actinobacillius pleuropneumoniae (AP) Amino acids in bold font correspond to AP amino acids F39, R177, H214, D215, M218, H219, Y222, H272, H277, S278, 1279, R281, M349, G370, H371, T438, T439, M440, K441, Q469, H495, P497, Y498, F517, N521, D525, and as shown in FIG. 45, correspond to identical amino acids in the MH and HD NGTs. FIG. 46A-B shows the structural alignment of these three NGTs, and illustrates the near identity in tertiary (i.e., structural and functional) configurations.

### Cell-Free Protein Synthesis (CFPS)

The components, systems, and methods disclosed herein may be applied to cell-free protein synthesis methods as known in the art. See, for example, U.S. Pat. Nos. 5,478,730; 5,556,769; 5,665,563; 6,168,931; 6,548,276; 6,869,774; 6,994,986; 7,118,883; 7,186,525; 7,189,528; 7,235,382; 7,338,789; 7,387,884; 7,399,610; 7,776,535; 7,817,794; 8,703,471; 8,298,759; 8,715,958; 8,734,856; 8,999,668; and 9,005,920. See also U.S. Published Application Nos. 2018/0016614, 2018/0016612, 2016/0060301, 2015-0259757, 2014/0349353, 2014-0295492, 2014-0255987, 2014-0045267, 2012-0171720, 2008-0138857, 2007-0154983, 2005-0054044, and 2004-0209321. See also U.S Published Application Nos. 2005-0170452; 2006-0211085; 2006-0234345; 2006-0252672; 2006-0257399; 2006-0286637; 2007-0026485; 2007-0178551. See also Published PCT International Application Nos. 2003/056914; 2004/013151; 2004/035605; 2006/102652; 2006/119987; and 2007/120932. See also Jewett, M. C., Hong, S. H., Kwon, Y. C., Martin, R. W., and Des Soye, B. J. 2014, “Methods for improved in vitro protein synthesis with proteins containing non-standard amino acids,” U.S. Patent Application Ser. No. 62/044,221; Jewett, M. C., Hodgman, C. E., and Gan, R. 2013, “Methods for yeast cell-free protein synthesis,” U.S. Patent Application Ser. No. 61/792,290; Jewett, M. C., J. A. Schoborg, and C. E. Hodgman. 2014, “Substrate Replenishment and Byproduct Removal Improve Yeast Cell-Free Protein Synthesis,” U.S. Patent Application Ser. No. 61/953,275; and Jewett, M. C., Anderson, M. J., Stark, J. C., Hodgman, C. E. 2015, “Methods for activating natural energy metabolism for improved yeast cell-free protein synthesis,” U.S. Patent Application Ser. No. 62/098,578. See also Guarino, C., & DeLisa, M. P. (2012). A prokaryote-based cell-free translation system that efficiently synthesizes glycoproteins. Glycobiology, 22(5), 596-601. The contents of all of these references are incorporated in the present application by reference in their entireties.

In some embodiments, a “CFPS reaction mixture” typically may contain a crude or partially-purified cell extract, an RNA translation template, and a suitable reaction buffer for promoting cell-free protein synthesis from the RNA translation template. In some aspects, the CFPS reaction mixture can include exogenous RNA translation template. In other aspects, the CFPS reaction mixture can include a DNA expression template encoding an open reading frame operably linked to a promoter element for a DNA-dependent RNA polymerase. In these other aspects, the CFPS reaction mixture can also include a DNA-dependent RNA polymerase to direct transcription of an RNA translation template encoding the open reading frame. In these other aspects, additional NTP's and divalent cation cofactor can be included in the CFPS reaction mixture. A reaction mixture is referred to as complete if it contains all reagents necessary to enable the reaction, and incomplete if it contains only a subset of the necessary reagents. It will be understood by one of ordinary skill in the art that reaction components are routinely stored as separate solutions, each containing a subset of the total components, for reasons of convenience, storage stability, or to allow for application-dependent adjustment of the component concentrations, and that reaction components are combined prior to the reaction to create a complete reaction mixture. Furthermore, it will be understood by one of ordinary skill in the art that reaction components are packaged separately for commercialization and that useful commercial kits may contain any subset of the reaction components of the invention.

The disclosed cell-free protein synthesis systems may utilize components that are crude and/or that are at least partially isolated and/or purified. As used herein, the term “crude” may mean components obtained by disrupting and lysing cells and, at best, minimally purifying the crude components from the disrupted and lysed cells, for example by centrifuging the disrupted and lysed cells and collecting the crude components from the supernatant and/or pellet after centrifugation. The term “isolated or purified” refers to components that are removed from their natural environment, and are at least 60% free, preferably at least 75% free, and more preferably at least 90% free, even more preferably at least 95% free from other components with which they are naturally associated.

As used herein, “translation template” for a polypeptide refers to an RNA product of transcription from an expression template that can be used by ribosomes to synthesize polypeptides or proteins.

The term “reaction mixture,” as used herein, refers to a solution containing reagents necessary to carry out a given reaction. A reaction mixture is referred to as complete if it contains all reagents necessary to perform the reaction. Components for a reaction mixture may be stored separately in separate container, each containing one or more of the total components. Components may be packaged separately for commercialization and useful commercial kits may contain one or more of the reaction components for a reaction mixture.

A reaction mixture may include an expression template, a translation template, or both an expression template and a translation template. The expression template serves as a substrate for transcribing at least one RNA that can be translated into a sequence defined biopolymer (e.g., a polypeptide or protein). The translation template is an RNA product that can be used by ribosomes to synthesize the sequence defined biopolymer. In certain embodiments the platform comprises both the expression template and the translation template. In certain specific embodiments, the reaction mixture may comprise a coupled transcription/translation (“Tx/Tl”) system where synthesis of translation template and a sequence defined biopolymer from the same cellular extract.

The reaction mixture may comprise one or more polymerases capable of generating a translation template from an expression template. The polymerase may be supplied exogenously or may be supplied from the organism used to prepare the extract. In certain specific embodiments, the polymerase is expressed from a plasmid present in the organism used to prepare the extract and/or an integration site in the genome of the organism used to prepare the extract.

Altering the physicochemical environment of the CFPS reaction to better mimic the cytoplasm can improve protein synthesis activity. The following parameters can be considered alone or in combination with one or more other components to improve robust CFPS reaction platforms based upon crude cellular extracts (for examples, S12, S30 and S60 extracts). The temperature may be any temperature suitable for CFPS. Temperature may be in the general range from about 10° C. to about 40° C., including intermediate specific ranges within this general range, include from about 15° C. to about 35° C., from about 15° C. to about 30° C., from about 15° C. to about 25° C. In certain aspects, the reaction temperature can be about 15° C., about 16° C., about 17° C., about 18° C., about 19° C., about 20° C., about 21° C., about 22° C., about 23° C., about 24° C., about 25° C.

The reaction mixture may include any organic anion suitable for CFPS. In certain aspects, the organic anions can be glutamate, acetate, among others. In certain aspects, the concentration for the organic anions is independently in the general range from about 0 mM to about 200 mM, including intermediate specific values within this general range, such as about 0 mM, about 10 mM, about 20 mM, about 30 mM, about 40 mM, about 50 mM, about 60 mM, about 70 mM, about 80 mM, about 90 mM, about 100 mM, about 110 mM, about 120 mM, about 130 mM, about 140 mM, about 150 mM, about 160 mM, about 170 mM, about 180 mM, about 190 mM and about 200 mM, among others.

The reaction mixture may include any halide anion suitable for CFPS. In certain aspects the halide anion can be chloride, bromide, iodide, among others. A preferred halide anion is chloride. Generally, the concentration of halide anions, if present in the reaction, is within the general range from about 0 mM to about 200 mM, including intermediate specific values within this general range, such as those disclosed for organic anions generally herein.

The reaction mixture may include any organic cation suitable for CFPS. In certain aspects, the organic cation can be a polyamine, such as spermidine or putrescine, among others. Preferably polyamines are present in the CFPS reaction. In certain aspects, the concentration of organic cations in the reaction can be in the general about 0 mM to about 3 mM, about 0.5 mM to about 2.5 mM, about 1 mM to about 2 mM. In certain aspects, more than one organic cation can be present.

The reaction mixture may include any inorganic cation suitable for CFPS. For example, suitable inorganic cations can include monovalent cations, such as sodium, potassium, lithium, among others; and divalent cations, such as magnesium, calcium, manganese, among others. In certain aspects, the inorganic cation is magnesium. In such aspects, the magnesium concentration can be within the general range from about 1 mM to about 50 mM, including intermediate specific values within this general range, such as about 1 mM, about 2 mM, about 3 mM, about 5 mM, about 6 mM, about 7 mM, about 8 mM, about 9 mM, about 10 mM, among others. In preferred aspects, the concentration of inorganic cations can be within the specific range from about 4 mM to about 9 mM and more preferably, within the range from about 5 mM to about 7 mM.

The reaction mixture may include endogenous NTPs (i.e., NTPs that are present in the cell extract) and or exogenous NTPs (i.e., NTPs that are added to the reaction mixture). In certain aspects, the reaction use ATP, GTP, CTP, and UTP. In certain aspects, the concentration of individual NTPs is within the range from about 0.1 mM to about 2 mM.

The reaction mixture may include any alcohol suitable for CFPS. In certain aspects, the alcohol may be a polyol, and more specifically glycerol. In certain aspects the alcohol is between the general range from about 0% (v/v) to about 25% (v/v), including specific intermediate values of about 5% (v/v), about 10% (v/v) and about 15% (v/v), and about 20% (v/v), among others.

The components, systems, and methods disclosed herein may be applied to recombinant cell systems (e.g., in vitro) and cell-free protein synthesis methods (e.g., in vivo) in order to prepare glycosylated proteins. In some embodiments, the methods, systems, and compositions may be performed using one or more in vivo steps. For example, prokaryotic or eukaryotic cells may be engineered to express one or more modified NGTs as disclosed herein, and/or to express one or more target polypeptides, wherein the target polypeptides comprise each an acceptor peptide sequence. The acceptor sequences may be the same for each target polypeptide or the acceptor peptide sequences may be different for each polypeptide. In some embodiments, the engineered NGT and the acceptor peptide sequence may be selected as a pair; that is, some engineered NGTs disclosed herein may have a stronger affinity and/or more efficient glycosylation activity with certain acceptor peptide sequences than others. Thus, in some embodiments, the target polypeptide may also be engineered to include a specific acceptor peptide sequence, or a target polypeptide, without engineering, may include a particular acceptor peptide sequence. In some embodiments, an engineered NGT has a stronger affinity and/or more efficient glycosylation activity for a wider range of different acceptor peptide sequences than the unmodified NGT counterpart. In some embodiments, the methods include one or more in vitro steps. For example, the modified NGTs and target peptides may be added to or combined in a cell-free method.

Glycosylated proteins that may be prepared using the disclosed components, systems, and methods may include proteins having N-linked glycosylation (i.e., glycans attached to nitrogen of asparagine). The glycosylated proteins disclosed herein may include unbranched and/or branched sugar chains composed of one or more monomers as known in the art such as glucose (e.g., β-D-glucose), galactose (e.g., β-D-galactose), mannose (e.g., β-D-mannose), fucose (e.g., α-L-fucose), N-acetyl-glucosamine (GlcNAc), N-acetyl-galactosamine (GalNAc), neuraminic acid, N-acetylneuraminic acid (i.e., sialic acid), and xylose, which may be attached to the glycosylated proteins, growing glycan chain, or donor molecule (e.g., a sugar donor nucleotide) via respective glycosyltransferases (e.g., N-glycosyltransferases). The glycosylated proteins disclosed herein may include glycans as known in the art including but not limited to Man₃GlcNAc₂ glycan, Man₅GlcNAc₃ glycan, and the fully sialylated human glycan Man₃GlcNAc₄Gal₂Neu₅Ac₂.

In certain exemplary embodiments, one or more of the methods described herein are performed in a vessel, e.g., a single, vessel. The term “vessel,” as used herein, refers to any container suitable for holding on or more of the reactants (e.g., for use in one or more transcription, translation, and/or glycosylation steps) described herein. Examples of vessels include, but are not limited to, a microtitre plate, a test tube, a microfuge tube, a beaker, a flask, a multi-well plate, a cuvette, a flow system, a microfiber, a microscope slide and the like.

### Glycosylation in Prokaryotes

Glycosylation in prokaryotes is known in the art. (See e.g., U.S. Pat. Nos. 8,703,471; and 8,999,668; and U.S. Published Application Nos. 2005/0170452; 2006/0211085; 2006/0234345; 2006/0252672; 2006/0257399; 2006/0286637; 2007/0026485; 2007/0178551; and International Published Applications WO2003/056914A1; WO2004/035605A2; WO2006/102652A2; WO2006/119987A2; and WO2007/120932A2; the contents of which are incorporated herein by reference in their entireties).

### Self-Assembled Monolayers for Matrix-Assisted Laser Desorption/Ionization Mass Spectrometry (SAMDI-MS)

The disclosed methods may utilize self-assembled monolayers for matrix-assisted laser desorption/ionization mass spectrometry (SAMDI-MS), for example, as a method for detecting glycosylation of peptides and proteins in the disclosed methods and systems. SAMDI-MS is known in the art and has been utilized to study peptides, proteins, and carbohydrates and their reaction products. (See Ban et al., “Discovery of Glycosyltransferases Using Carbohydrate Arrays and Mass Spectrometry,” Nat. Chem. Biol., 2012, 8, 769-773; Ban et al., “On-Chip Synthesis and Label-Free Assays of Oligosaccharide Arrays,” Chem. Int. Ed., 2008, 47(18), 3396-3399; Houseman et al., “Maleimide-Functionalized Self-Assembled Monolayers for the Preparation of Peptide and Carbohydrate Biochips,” Langmuir, 2003, 19(5), 1522-1531; Su et al., “Using Mass Spectrometry to Characterize Self-Assembled Monolayers Presenting Peptides, Proteins and Carbohydrates,” Angew. Chem. Int. Ed., 2002, 41, 4715-4718; Houseman et al., “Toward Quantitative Assays with Peptide Chips: A Surface Engineering Approach,” Trends Biotech., 2002, 20 (7), 279-281; Houseman et al., “Carbohydrate Arrays for the Evaluation of Protein Binding and Enzyme Activity,” Chem. Biol., 2002, 9, 443-454); and Laurent, N., et al. (2008). “Enzymatic Glycosylation of Peptide Arrays on Gold Surfaces.” Chembiochem 9(6): 883-887); the contents of which are incorporated herein by reference in their entireties).

### Advantages

Exemplary, non-limiting advantages of the compositions and methods disclosed herein is provided below.

Most methods for glycoprotein synthesis use eukaryotic organisms. Currently approved glycoprotein therapeutic are produced using OST-based glycosylation systems within mammalian or yeast cells. Bacterial and in vitro glycosylation systems offer the opportunity to more closely control glycosylation patterns and more rapidly develop more diverse glycosylation systems. Most existing methods use a membrane bound oligosaccharyltransferase (OST) to transfer lipid-linked sugar donors en bloc onto proteins.

NGTs are soluble enzymes which transfer sugars from activated donors directly onto proteins without the use of membrane bound components. However, they have not yet been widely adopted for the modification of heterologous proteins, likely due to differences in their peptide acceptor and sugar donor specificities compared to human OST-based glycosylation systems. Thus far, the use of NGTs for modification of recombinant proteins has generally required engineered the target sequence and therefore an inability to design efficiently modified glycosylation sites. The broad goal of this work is to develop a repertoire of engineered NGTs capable of glycosylating any sequence of interest, alleviating or diminishing the need to alter the primary amino acid sequences of target proteins for naturally occurring glycosylation sites.

Two studies by Naegali et. al. in 2014 showed that NGT can efficiently modify some N-X-S/T motifs with glucose, galactose, xylose, or mannose and showed trends of modification in living cells. This study also showed that ApNGT can modify wildtype human erythropoietin in the E. coli cytoplasm (although protein solubility and glycosylation efficiency was not determined). Other work by the Aebi lab disclosed in a patent showed modification of wildtype bacterial autotransporter proteins (native substrates for NGTs) in cells and their potential use as a vaccine.

In 2017, a work by Qitao Song et al entitled “Production of homogeneous glycoprotein with multisite modifications by an engineered N-glycosyltransferase mutant” discovered a mutant of ApNGT (Q469A) that exhibits increased promiscuity and activity compared to the wildtype. Unfortunately, the inventors have found that this mutant is still only able to modify 45% of possible 4-mer glycosylation sequences of the form X-N-X-S/T efficiently (>80% modification) and 80% of sequences inefficiently (>5% modification). In this work, the inventors carry out further engineering of the ApNGT (Q469A) enzyme (SEQ ID NO: 1a) and find that the additional mutations that the inventors introduce allow for more than 66% of possible 4-mer glycosylation sequences efficiently (>80% modification) and 93% of sequences inefficiently (>5%).

By discovering NGTs with new specificities, the other problem that the invention overcomes is the site-specific installation of unique sugars at different N-linked glycosylation sites within a single recombinant protein. There have been many instances of installing unique glycans on peptides as they can be assembled by Fmoc Solid phase synthesis using glycosylated amino acids as substrates. However, site-specifically controlling glycosylation on multiple sites within a biologically produced protein has proved more difficult. Most efforts in this area have been lead by Prof Benjamin Davis and colleagues at the University of Oxford and have focused on chemically modifying naturally occurring or non-canonical amino acids within the protein using biorthogonal or semi-biorthogonal chemistries. Another set of papers in this area led by Prof Lai-Xi Wang at the University of Maryland demonstrated the use of glycosidases to direct site-specific glycosylation of antibodies (PNAS, 2018) and erythopoeitin (ACS Chem Biol, 2017).

The NGTs described in this work would provide a greatly increased set of enzyme specificities and therefore potential orthogonalities to implement a sequential glycosylation strategy.

Additional, non-limiting advantages of the compositions and methods disclosed herein include the following:

NGT glycosylation systems allow for efficient modification of polypeptides without a eukaryotic host, lipid-bound substrates and enzymes, or the need for transport to the bacterial periplasm (as is required to use existing oligosaccharyltransferase glycosylation methods). Previously this system was only able to modify a limited set of acceptor peptide sequences and generally required the modification of natural protein sequences to enable efficient modification. The current findings increase the set of sequences that can be efficiently modified such that natural protein sequences do not need to be altered. These engineered NGTs can be applied to achieve glycosylation in vitro or in living cells.

Demonstrated improved glycosylation of several therapeutic proteins (including IFN-gamma, GM-CSF, and human IgG Fc fragment) in E. coli cytoplasm and in vitro with its native glycosylation sequence by using an engineered NGT.

Demonstrated installation of two distinct glycans on a single protein using distinct N-glycosyltransferase specificities.

The method using cell-free protein synthesis from linear templates followed by SAMDI-MS analysis allowed for the rapid synthesis and characterization of many enzyme variants and variant pools with >50,000 reactions. Current studies of glycosyltransferase specificity require expression and purification of the enzyme from cells by affinity purification, screening by incorporation of radioactively or chemically labeled sugars or liquid chromatography (LC) methods, and validation by mass spectrometry (typically LC-MS). These methods limit investigations to 10-100 peptides.

The findings disclosed herein allow for site-specific and efficient enzymatic N-linked glycosylation of native therapeutic proteins in vitro and in the bacterial cytoplasm by using engineered N-glycosyltransferases. This technique could enable quicken development and reduce production costs for glycoprotein therapeutics. The method the inventors developed using SAMDI-MS and CFPS can rapidly recapitulate these results for other enzymes homologs or enzyme variants of interest.

### Applications

Non-limiting applications of the compositions and methods disclosed herein include the following:

Design of therapeutic polypeptide amino acid sequences for improved glycosylation by an engineered N-glycosyltransferase in vitro or in a cell;

High-throughput engineering glycosyltransferases for alternative sugar donor or acceptor peptide specificities;

Production of high titers of proteins (such as therapeutics) in industrial bacterial host organisms which are glycosylated site-specifically in the bacterial cytoplasm without the need to alter primary amino acid sequences;

In vitro glycosylation of proteins produced in living cells without the need to alter their primary amino acid sequences;

Bacterial production or in vitro glycosylation of proteins (such as therapeutics) that do not naturally contain canonical N-glycosylation sequences without the need to alter their primary amino acid sequences;

Use of engineered N-glycosyltransferases to glycosylate proteins within eukaryotic cell cytoplasm or as an orthogonal glycosylation method to eukaryotic N-linked glycosylation;

Our engineered NGTs possess a broader range of peptide acceptor specificities than those found in nature. This can be used to glycosylate proteins that could not otherwise be glycosylated in bacterial or in vitro systems. Differences and orthogonalities between the NGT specificities can also be used to site-specifically install distinct glycans onto multiple locations within a single protein by sequential treatment with engineered or natural N-glycosyltransferases with intervening elaboration steps;

Isolation of proteins modified at a subset of all canonical glycosylation sites using engineered or natural N-glycosyltransferases.

This invention allows for the production of glycosylated proteins in vitro or within bacterial systems without modifying their native amino acid sequences. glycosylated proteins, including protein therapeutics and vaccines. The lipid-independent nature of this system makes it attractive for in vitro modification of protein therapeutics and glycosylation in the bacterial cytoplasm. These high-titer, rapid expression systems could allow glycoprotein therapeutics to be developed and produced more quickly and at lower cost.

The invention also allows for the site-specific installation of distinct glycans at multiple locations within a single protein. This could be used, for example, to install immunomodulatory glycans to glycoconjugate vaccines or to simply optimize glycosylation structures at multiple locations (such as a Fab or Fc region of an IgG antibody).

The invention identifies enzymes, especially engineered enzymes, and sequences for site-specific glycosylation.

Additional non-limiting applications further include:

Use of modified enzymes (engineered N-glycosyltransferase (NGTs) mutants) for the modification of target proteins.

Coexpression of polypeptides and modified enzymes (engineered N-glycosyltransferase (NGTs) mutants) in a living cell.

Use of polypeptide sequences and modified enzymes (engineered N-glycosyltransferase (NGTs) mutants) as a means of glycosylation in vitro.

Method for rapid engineering of the peptide and sugar specificities of mutated N-glycosyltransferase peptide and sugar specificity using Self-Assembled Monolayers for Desorption Ionization Mass Spectrometry (SAMDI-MS).

Use of modified enzymes (engineered N-glycosyltransferase (NGTs) mutants) and methods for rapid engineering describe above where NGTs are synthesized by cell-free protein synthesis.

Use of modified enzymes (engineered N-glycosyltransferase (NGTs) mutants) and methods for rapid engineering describe above to characterize and obtain enzymes with new specificities.

Use of modified enzymes (engineered N-glycosyltransferase (NGTs) mutants) to install alternative monosaccharides such as glucose, galactose, and N-glucosamine.

Sequential modification of proteins using unique peptide acceptor specificities of naturally occurring or engineered NGTs to install multiple, distinct glycans on a single protein or to direct modification to a subset of all present N-glycosylation sites.

### Miscellaneous

The steps of the methods described herein can be performed in any suitable order unless otherwise indicated herein or otherwise clearly contradicted by context. The steps may be repeated or reiterated any number of times to achieve a desired goal unless otherwise indicated herein or otherwise clearly contradicted by context.

Preferred aspects of this invention are described herein, including the best mode known to the inventors for carrying out the invention. Variations of those preferred aspects may become apparent to those of ordinary skill in the art upon reading the foregoing description. The inventors expect a person having ordinary skill in the art to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### ILLUSTRATIVE EMBODIMENTS

The following Embodiments are illustrative and should not be interpreted to limit the scope of the claimed subject matter.

Embodiment 1. A modified N-glycosyltransferase (NGT) selected from the following group or a homolog thereof: (i) an Actinobacillus spp. (optionally a modified NGT of Actinobacillus pleuropneumoniae of SEQ ID NO:1) comprising one or more substitutions at amino acid positions F39, R177, H214, D215, M218, H219, Y222, H272, H277, S278, 1279, R281, M349, G370, H371, T438, T439, M440, K441, A469 (Q469), H495, P497, Y498, F517, N521, and D525; (ii) a Kingella spp. (optionally a modified NGT of Kingella kingae of SEQ ID NO:2) comprising one or more substitutions at amino acid positions F42, R181, H218, D219, M222, H219, Y223, H276, H281, S282, 1283, R285, M354, G375, H376, T443, T444, M445, K446, A474 (Q474), H500, P502, Y503, F522, N526, and D530; (iii) a Haemophilus spp. (optionally a modified NGT of Haemophilus pneumoniae of SEQ ID NO:3) comprising one or more substitutions at amino acid positions F68, R204, H241, D242, M245, H246, Y249, H298, H303, S304, 1305, R307, M375, G396, H397, T464, T465, M466, K467, A495 (Q495), H521, P523, Y524, F543, N547, and D551; (iv) nan Aggregatibacter spp. (optionally a modified NGT ofAggregatibacter aphrophilus of SEQ ID NO:4) comprising one or more substitutions at amino acid positions F39, R177, H214, D215, M218, H219, Y222, H270, H275, S276, 1277, R279, M348, G369, H370, T437, T438, M439, K440, A468 (Q468), H494, P496, Y497, F516, N520, and D524; (v) a Mannheimia spp. (optionally a modified NGT of Mannheimia haemolytica of SEQ ID NO:5) comprising one or more substitutions at amino acid positions F39, R177, H214, D215, M218, H219, Y222, H272, H277, S278, 1279, R281, M349, G370, H371, T438, T439, M440, K441, A469 (Q469), H495, P497, Y498, F517, N521, and D525; (vi) a Bibersteinia spp. (optionally a modified NGT of Bibersteinia trehalosi of SEQ ID NO:6) comprising one or more substitutions at amino acid positions F40, R180, H217, D218, M221, H222, Y225, H274, H279, S280, 1281, R283, M351, G372, H373, T440, T441, M442, K443, A471 (Q471), H497, P499, Y500, F519, N523, and D527; and (vii) a Haemophilus spp. (optionally a modified NGT of Haemophilus ducreyi of SEQ ID NO:7) comprising one or more substitutions at amino acid positions F38, R176, H213, D214, M217, H218, Y221, H271, H276, S277, 1278, R280, M348, G369, H370, T437, T438, M439, K440, A468 (Q468), H494, P496, Y497, F516, N520, and D524.

Embodiment 2. The modified NGT of embodiment 1, wherein the amino acid substitution is at one or more positions, with reference to SEQ ID NO:1, selected from the group consisting of H219, T438, A696 and H495 or a homologous position thereof.

Embodiment 3. The modified NGT of embodiment 1, wherein the amino acid substitution is at one or more positions, with reference to SEQ ID NO:1, selected from the group consisting of: H219F or H219W; T438S or T438E; A696G or A696I; and H495D, or a homologous position thereof.

Embodiment 4. The modified NGT of embodiment 1, wherein the wild-type NGT comprises any of SEQ ID NOs:1-7 and the modified NGT comprises at least one substitution mutation, at a position with reference to SEQ ID NO:1, selected from the group consisting of: H219F, H219W, T438S, T439E, A469G, A469I, H495D, H219F-T438S, H219F-H495D, H219W-T438S, H219W-H495D, A469G-H495D, and A469I-H495D, or a homologous position thereof, wherein H219F-T438S, H219F-H495D, H219W-T438S, H219W-H495D, A469G-H495D, and A469I-H495D is a combination of two substitution mutations.

Embodiment 5. The modified NGT of any of the foregoing embodiments, wherein the modified NGT glycosylates a wider array of acceptor peptide sequences as compared to an unmodified NGT under the same reaction conditions.

Embodiment 6. The modified NGT of any of the foregoing embodiments, wherein the modified NGT has an affinity for a wider array of acceptor peptide sequences as compared to an unmodified NGT under the same reaction conditions.

Embodiment 7. The modified NGT of any of the foregoing embodiments, wherein the acceptor peptide sequence comprises the amino acid sequence [X₋₂-]-[X₋₁-]-[N]-[X₊₁]-[X₊₁]-[X₊₂]-[X₊₃], where X is any canonical amino acid, and optionally where [X₊₁] is not P.

Embodiment 8. The modified NGT of any of the foregoing embodiments, wherein the acceptor peptide sequence comprises the amino acid sequence [X₋₂]-[X₋₁-]-[N]-[X₊₁]-[X₊₁]-[X₊₂]-[X₊₃], where X is any canonical amino acid, and optionally where [X₊₁] is not P, and optionally where [X₊₂] is not S or T.

Embodiment 9. A polynucleotide sequence encoding the modified NGT of any of the foregoing embodiments.

Embodiment 10. An expression vector comprising the polynucleotide sequence of embodiment 9.

Embodiment 11. A bacterial cell comprising the modified NGT of any of embodiments 1-8, the polynucleotide sequence of embodiment 9, or the expression vector of embodiment 10.

Embodiment 12. The bacterial cell of embodiment 11, further comprising a target polypeptide.

Embodiment 13. A eukaryotic cell comprising the modified NGT of any of embodiments 1-8, the polynucleotide sequence of embodiment 9, or the expression vector of embodiment 10.

Embodiment 14. The eukaryotic cell of embodiment 13, further comprising a target polypeptide.

Embodiment 15. A method for glycosylating a target polypeptide, wherein the target polypeptide comprises an acceptor peptide sequence, the method comprising: contacting the target polypeptide with the modified NGT of any of embodiments 1-8 and a glycan under suitable reaction conditions.

Embodiment 16. The method of embodiment 15, wherein the target polypeptide comprises a therapeutic polypeptide.

Embodiment 17. The method of embodiment 15 or 16, wherein the method is performed in vivo.

Embodiment 18. The method of embodiment 15 or 16, wherein the method is performed in vitro.

Embodiment 19. The method of any of embodiments 15-18, wherein the target protein is a prokaryotic protein.

Embodiment 20. The method of any of embodiments 15-18, wherein the target protein is a eukaryotic protein.

Embodiment 21. The method of any of embodiments 15-20, wherein the target protein comprises an acceptor peptide sequence comprising the amino acid sequence [X₋₂]-[X₋₁-]-[N]-[X₊₁]-[X₊₁]-[X₊₂]-[X₊₃], where X is any canonical amino acid, and optionally where [X₊₁] is not P, and optionally where [X₊₂] is not S or T.

Embodiment 22. The method of any of embodiments 15-21, wherein the NGT glycosylates the target polypeptide with one or more glycans.

Embodiment 23. The method of any of embodiments 15-22, wherein the glycan comprises one or more monosaccharides selected from the group consisting of glucose, galactose, and N-glucosamine.

Embodiment 24. A modified N-glycosyltransferase (NGT) comprising one or more substitutions at amino acid positions corresponding to Actinobacillus pleuropneumoniae NGT of SEQ ID NO:1: F39, R177, H214, D215, M218, H219, Y222, H272, H277, S278, 1279, R281, M349, G370, H371, T438, T439, M440, K441, A469, H495, P497, Y498, F517, N521, and D525.

Embodiment 25. The modified NGT of embodiment 24, wherein the NGT is derived from an organism selected from the group consisting of: Kingella kingae; Haemophilus influenzae; Aggregatibacter aphrophilus; Mannheimia haemolytica; Bibersteinia trehalosi; Haemophilus ducreyi; Burkholderia sp; Yersinia enterocolitia; Yersinia pestis; Salmonella enterica; and Escherichia coli.

Embodiment 26. The modified NGT of embodiment 24, wherein the NGT is derived from an organism selected from the group consisting of: Mannheimia haemolytica and Haemophilus ducreyi.

Embodiment 27. The modified NGT of any one of embodiments 24-26, wherein the modified NGT glycosylates a wider array of acceptor peptide sequences as compared to an unmodified NGT under the same reaction conditions.

Embodiment 28. The modified NGT of any one of embodiments 24-26, wherein the modified NGT has an affinity for a wider array of acceptor peptide sequences as compared to an unmodified NGT under the same reaction conditions.

Embodiment 29. A therapeutic composition comprising the therapeutic peptide of embodiment 16.

Embodiment 30. The therapeutic composition of embodiment 29, wherein the composition comprises a vaccine.

### EXAMPLES

The following Examples are illustrative and are not intended to limit the scope of the claimed subject matter.

### Example 1—Using High Throughput Analysis to Engineer N-Glycosyltransferases with Altered Specificities

### A. Introduction

N-Linked protein glycosylation is the modification of asparagine side chains with complex oligosaccharides and is among the most common and complex post-translational modification (PTM) found in nature¹. In eukaryotes, N-glycans are installed at the canonical sequence motif N-X-S/T (where X≠P)². The majority of protein therapeutics are N-glycosylated³ and differences in glycosylation pattern are known to have strong effects on bioactivity^{4, 5}, protein stability⁶, and serum half-life⁷. The introduction of additional N-glycosylation sites into therapeutic proteins has also been shown to improve therapeutic properties, including prolonged serum half-life^{8, 9}. While the use of mammalian cell lines with endogenous N-glycosylation pathways is the most common method to produce glycoprotein therapeutics, these constitutive systems limit the diversity of glycan structures that can be constructed^{10, 11} and often suffer from heterogeneity of the glycoprotein products^{3, 12, 13}. These limitations have motivated the development of synthetic glycosylation systems in Escherichia coli (E. coli) or in vitro to install^{10, 14-18} or remodel^{12,16} defined glycans to precisely control the structures and properties of glycoproteins.

Among these synthetic glycosylation systems, a class of bacterial cytoplasmic enzymes known as N-glycosyltransferases (NGTs) are important for glycoengineering because they can efficiently transfer a single glucose residue from a uracil-diphosphate-glucose (UDP-Glc) sugar donor onto certain native eukaryotic sequences^{19, 20}. This glucose residue can then be extended into a full-length glycan using glycosyltransferases²¹ or endoglycosidase chemoenzymatic glycan remodeling^{22, 23}. Rigorous analyses of NGT specificities have shown that NGTs can only modify a fraction of all possible eukaryotic N-glycosylation sequences^{19, 22}. Because there is a continuously expanding set of potential therapeutic protein targets that could be optimized by glycoengineering—including proteins without the canonical N-X-S/T glycosylation sequences—there is a clear need to engineer or discover NGTs that enable the modification of an expanded set of acceptor sequences. The broad goal of this work is to develop a repertoire of NGTs capable of glycosylating any sequence of interest, alleviating the need to alter the primary amino acid sequences of target proteins for naturally occurring glycosylation sites or introducing new glycosylation sites.

Approaches based on traditional directed evolution are effective for engineering enzyme activity towards a single substrate but are not yet suited to developing enzymes that display multiple functions (i.e., distinct peptide specificities). Wells and collaborators addressed this limitation by developing a method to engineer peptide ligases by identifying modified substrates from the E. coli proteome using a liquid-chromatography tandem mass spectrometry (LC-MS/MS) proteomics approach²⁴. While proteomic identification works well for ligases and proteases²⁵, it is difficult to apply to glycosylation enzymes because enrichment methods for glycopeptides are not generalizable and differences in substrate peptide length strongly affect glycosylation efficiency¹⁹. Furthermore, proteomic identification provides some information on substrate preferences, but does not directly measure activity. The inventors have developed a general and versatile assay called self-assembled monolayers for matrix-assisted laser desorption/ionization mass spectrometry (SAMDI-MS), which can rapidly and quantitatively measure enzymatic specificities and activities on a large number of substrates without the need to purify enzymes or substrates^{19, 26, 27}. The inventors have recently combined this method with cell-free protein synthesis (CFPS) of enzymes to create the GlycoSCORES workflow, which the inventors used to analyze the specificity of several NGTs^{19, 22}.

Here the inventors disclose the use of the GlycoSCORES workflow with high throughput CFPS reactions from PCR-derived linear expression templates (LET-CFPS)²⁸ to develop a panel of NGTs that significantly expands the range of sequences that can be directly glycosylated. The parallel workflow to develop this panel relies on two key steps. First, the inventors screened acceptor sequence specificity on pools of 26 site-saturated variant libraries (SSVLs) of the parent NGT. Each of the SSVLs is comprised of 19 mutants at a specific residue that was targeted for mutagenesis based on inspection of the NGT crystal structure and expected interactions with the substrate peptide. By screening these SSVLs on substrate peptide libraries, the inventors separately identified residues that determine specificity at the X₋₂, X₋₁, X₊₁, X₊₂, and X₊₃ positions of the substrate peptide, relative to the glycosylated asparagine. Second, the inventors generated and rigorously characterized precise, single or double mutants that, collectively, expand the set of canonical (N-X-S/T where X≠P) and non-canonical (N-X-Z where X≠P and Z≠S/T) peptide sequences that can be efficiently modified compared to parent NGT alone. The inventors discovered 13 NGT mutants, in addition to the parent NGT, which significantly increase the fraction of all X₋₁ and X₊₁ canonical sequence combinations (684 in total) that can be modified with high efficiency—in yields of approximately 45% to 65%. Another panel of NGTs allow for modification of a variety of sequences with the X₊₂ positions besides S/T (10 of 17 amino acids, e.g., Ala, Asp, Met and Val). Moreover, the inventors demonstrated the utility of the NGT mutant panel by increasing the modification efficiency of approved therapeutic proteins, compared to the parent NGT, without modifying their amino acid sequences. The inventors expect that this method will be helpful in the development of additional enzymes with altered specificities, and the inventors anticipate that the NGT mutants discovered here will significantly expand the application areas for bacterial and in vitro glycoengineering.

### B. Results

### 1. Identifying Specificity-Determining Residues by Site-Saturated Library Screening

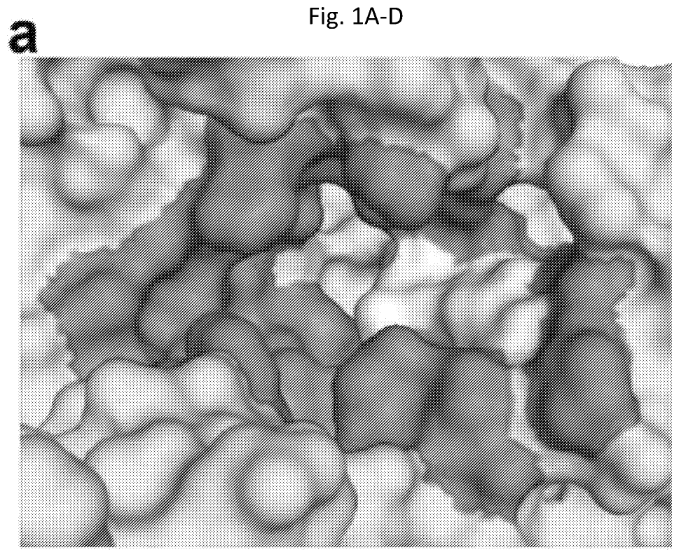

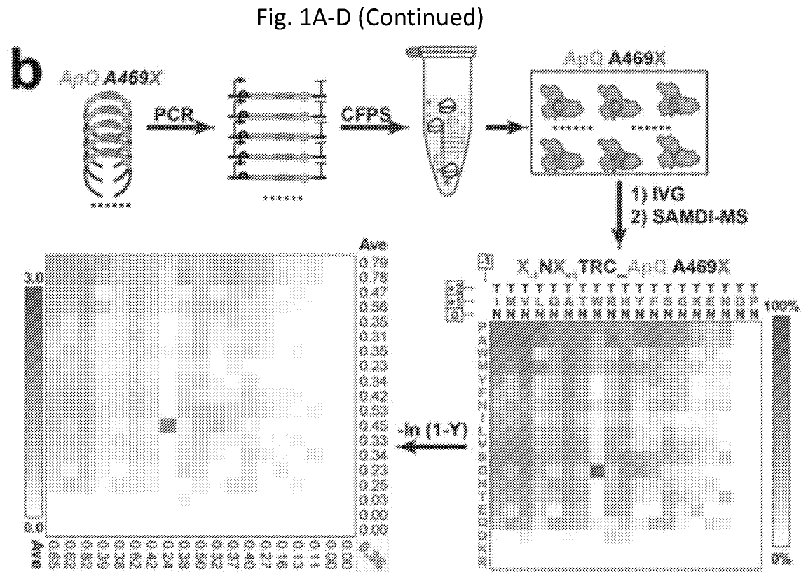

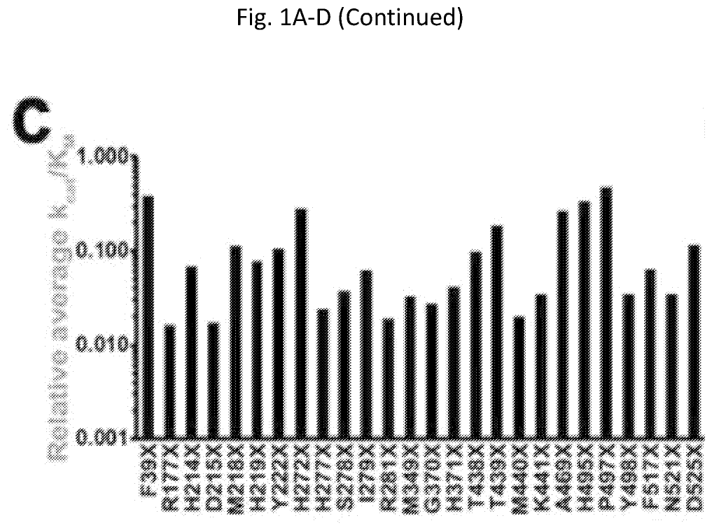

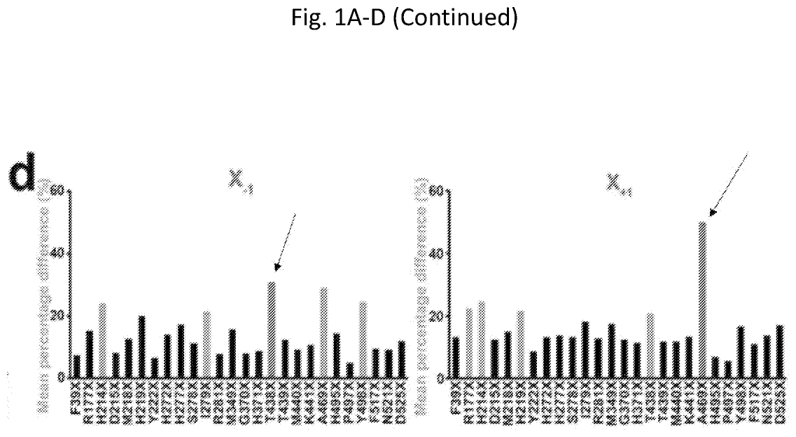

To develop a panel of mutant NGT enzymes capable of efficiently modifying a broad range of defined protein glycosylation sites, the inventors first set out to identify the residues that directly determine substrate specificity. While a crystal structure of the NGT complex with the substrate could provide this information, the known crystal structure of NGT²⁹ from Actinobacillus pleuropneumoniae (ApNGT) does not provide the location of the acceptor peptide, only showing the uracil-diphosphate (UDP) portion of the UDP-Glc sugar donor. Therefore, it was necessary to first identify residues that determine specificity by directly screening enzyme mutants. To that end, the inventors selected 26 residues surrounding the UDP-binding pocket of ApNGT for mutagenesis (FIG. 1a). The inventors then ordered fully saturated libraries for each of these residues as linear DNA, using a previously reported Q469A mutant of ApNGT (we refer to this parent mutant as ApQ)^{20, 30} as a starting point because it has much higher activity than wildtype ApNGT for its peptide substrates. Each of these SSVLs contained DNA encoding enzymes with an approximately equal mixture of the 19 non-wildtype amino acids (indicated by an “X”) at one of the 26 targeted residues. In this way, the inventors test each library as a pool, rather than individual clones, to identify residues having the greatest impact on activity and peptide specificity.

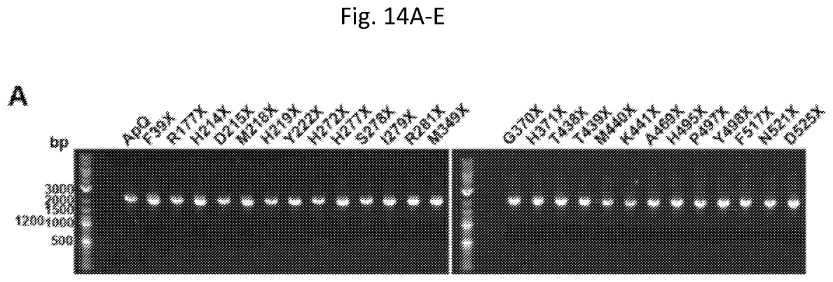

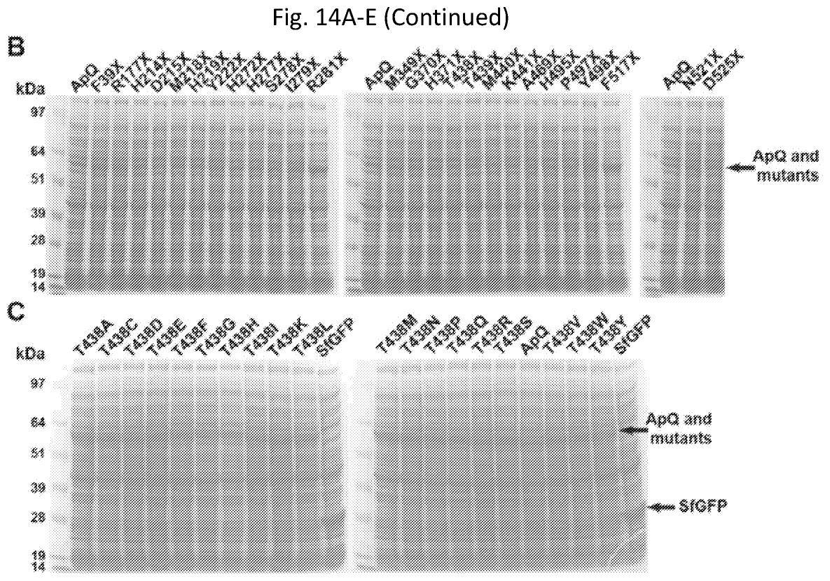

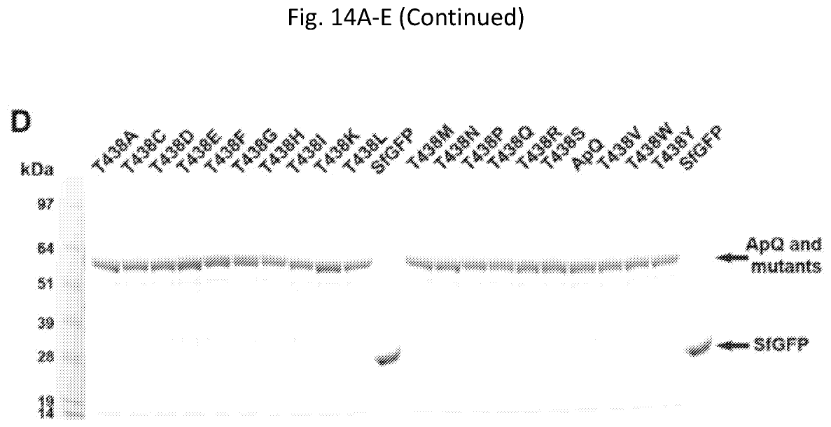

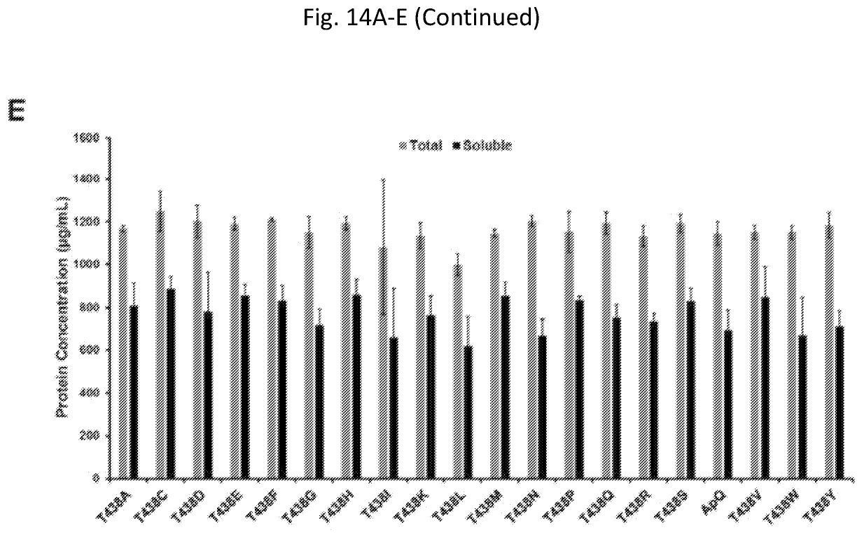

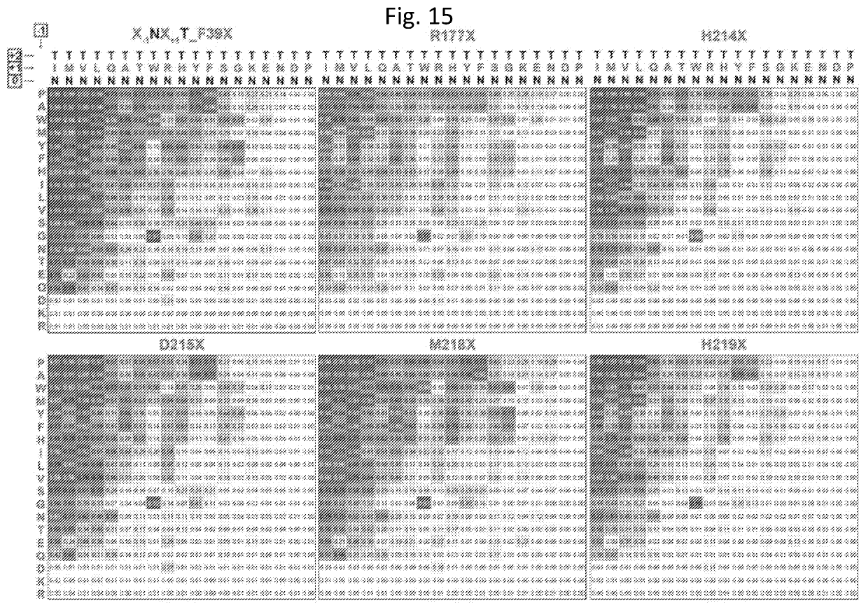

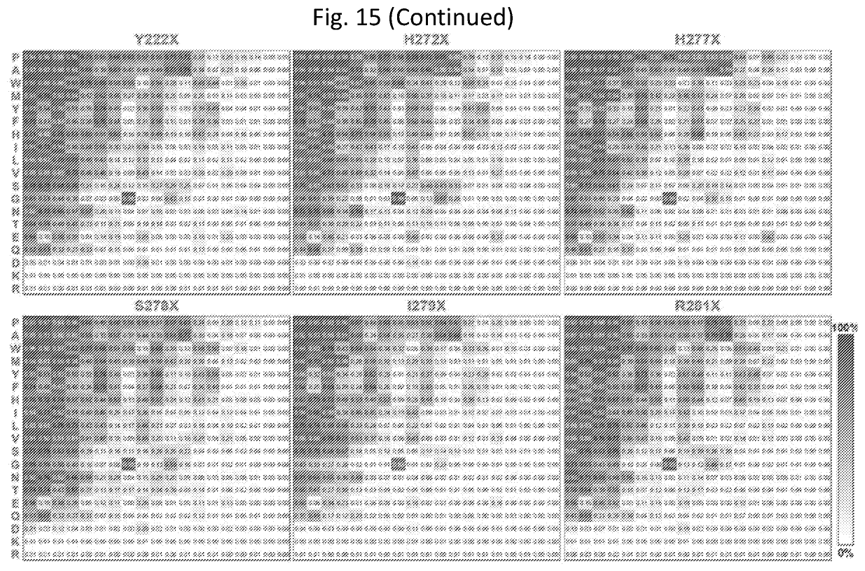

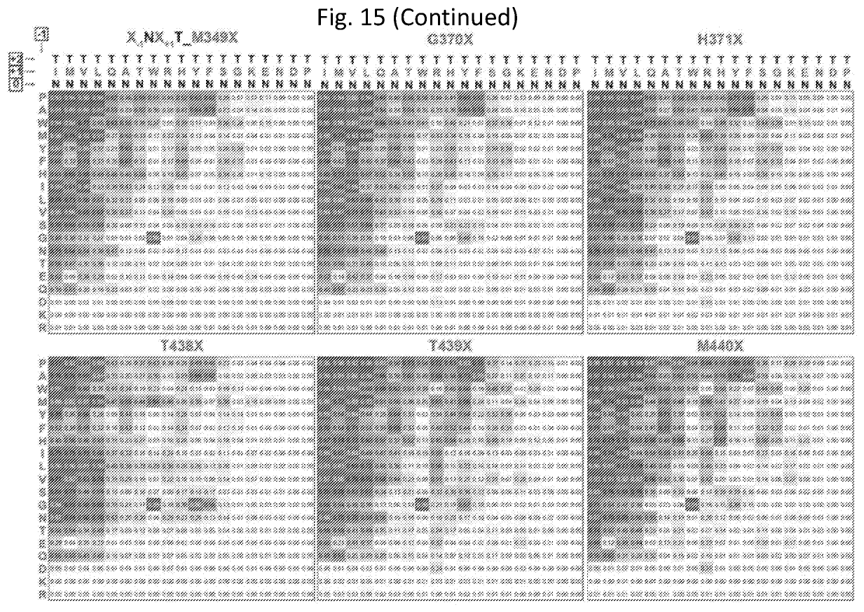

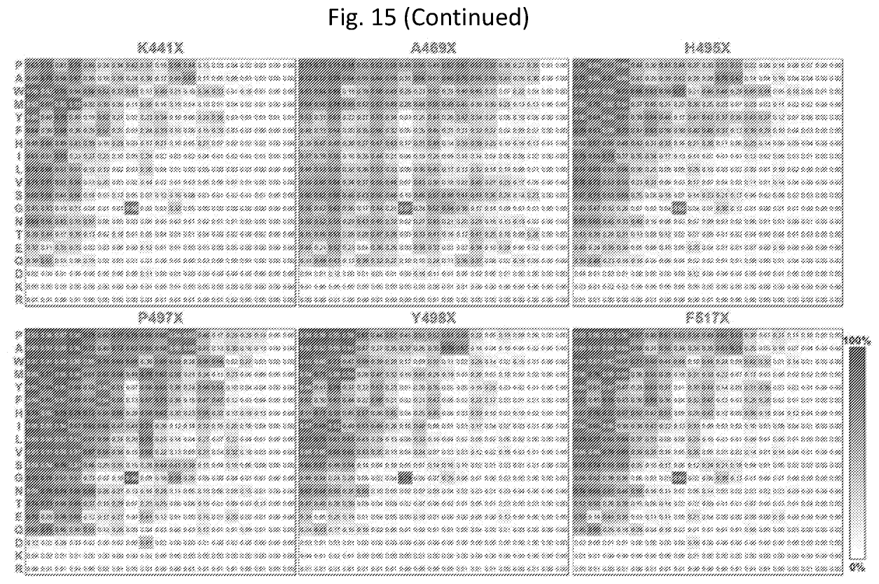

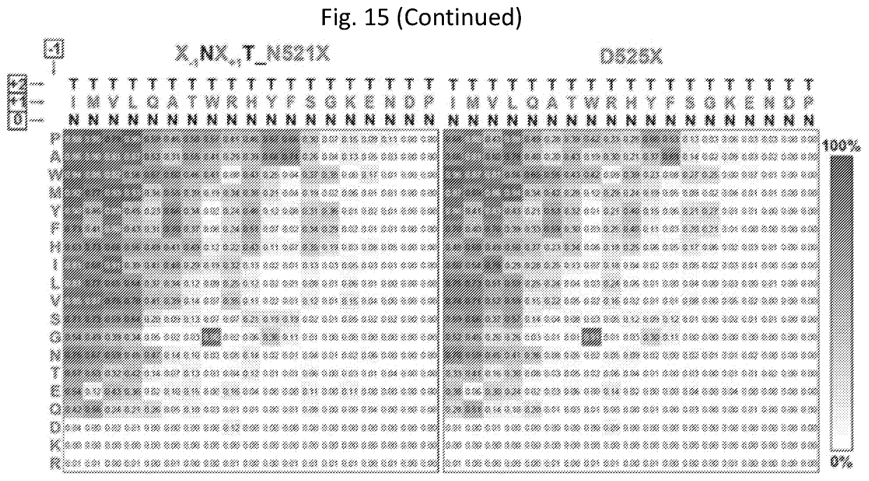

The inventors performed PCR on each of these SSVLs and directly used the resulting linear expression templates (LETs) to drive expression of protein SSVLs in CFPS (FIG. 1b). All 26 SSVLs were expressed at similar levels compared to ApQ (FIG. 14A-E). All 26 protein SSVLs as well as the parent ApQ were used directly in glycosylation assays of each peptide in a 361-member substrate library with the motif X₋₁-N-X₊₁-TRC where X₋₁ and X₊₁ are one of the 19 amino acids (Cys excluded). After in vitro glycosylation (IVG), peptides and glycopeptides were covalently pulled down onto maleimide-functionalized self-assembled monolayers by reaction with the C-terminal cysteine and then analyzed with SAMDI-MS for peptide modification (modification heatmaps shown in FIG. 15).

To compare the differences between ApQ and each of the SSVLs, the inventors first calculated the average activity across the entire peptide library. The inventors used concentrations of peptide substrates that were generally 10-fold lower than the K_{M} for most peptide and NGT combinations^{19, 20}, and were therefore able to compare the approximate k_{cat}/K_{M} of each reaction using the equation −ln(1−Y)=k_{cat} K_{M}*c*t, where c is enzyme concentration, t is reaction time, and Y is yield of modification. The inventors converted each modification data point within each of heatmaps for the 26 protein SSVLs to generate heatmaps showing −ln(1−Y) (FIG. 1b) and then obtained an approximated average k_{cat}/K_{M} value for each SSVL across all 361 peptides. While this quantification method does not provide an exact k_{cat}/K_{M} value for each enzyme-substrate combination (doing so would require tens of separate measurements for each substrate), it does allow one to present and compare one approximate value of average k_{cat}/K_{M} for each SSVL based on 361 data points for each peptide substrate in the library. To enable comparison, the average k_{cat}/K_{M} of each SSVL was then normalized to that of ApQ (FIG. 1c, see Methods for more details). As might be expected, no SSVL showed greater activity—measured as an average across all 361 peptides—than the parent ApQ. The inventors observed that the R177X, D215X, R281X, and M440X SSVLs had the poorest average activities (less than 2% relative to ApQ), indicating that no individual mutant in these SSVLs provides activity close to that of ApQ and that these residues may likely be important to catalysis or substrate (peptide receptor or sugar donor) binding of ApQ.

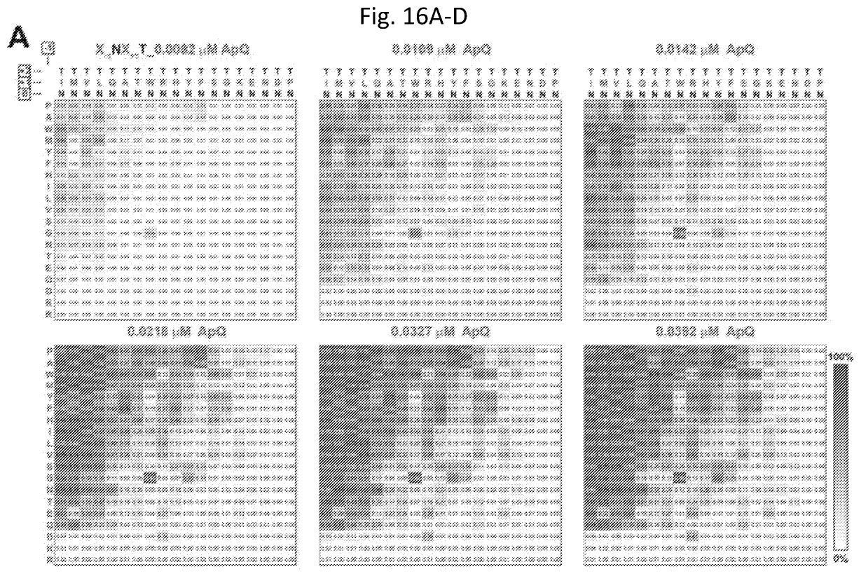

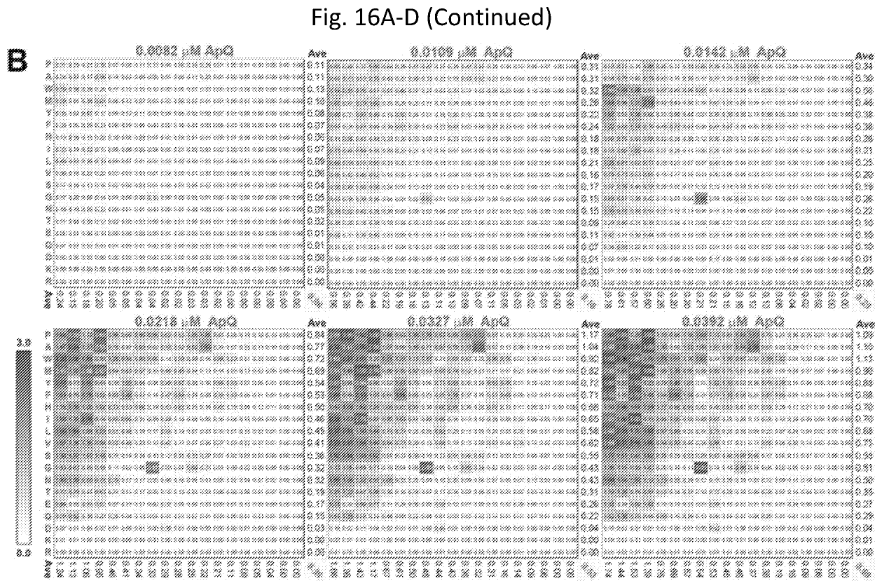

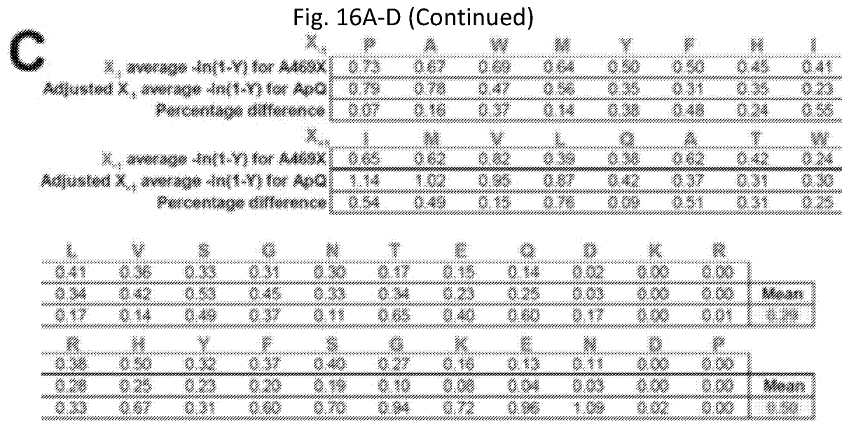

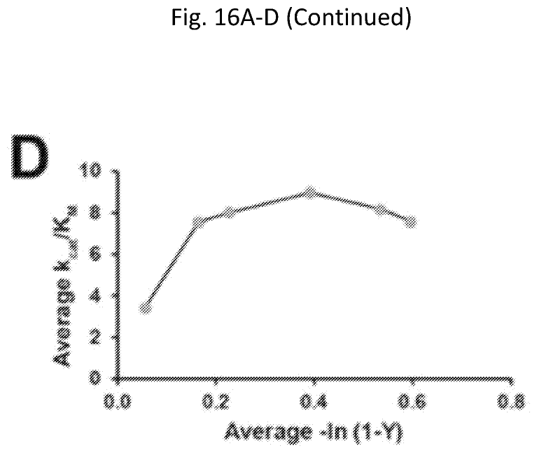

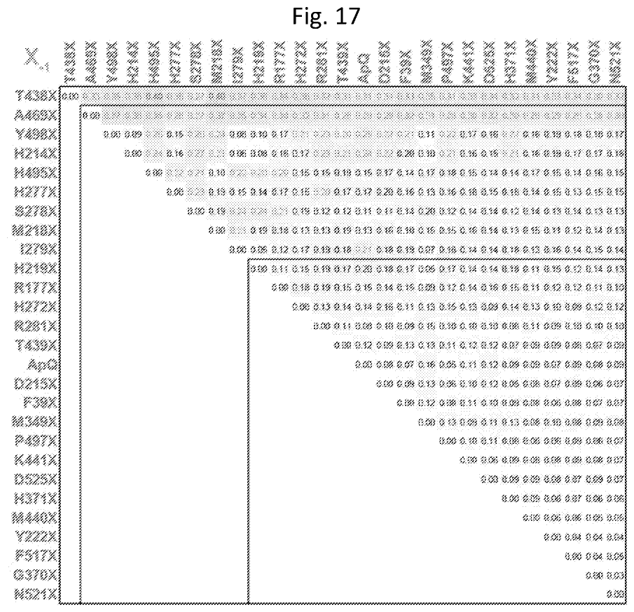

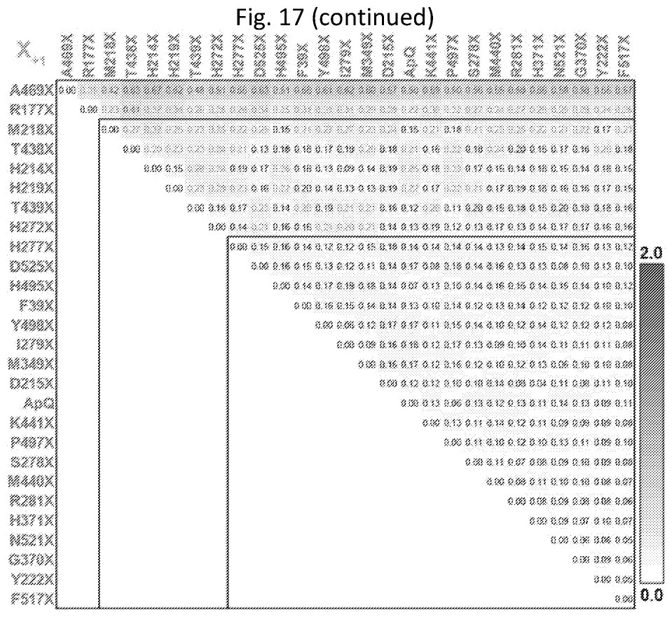

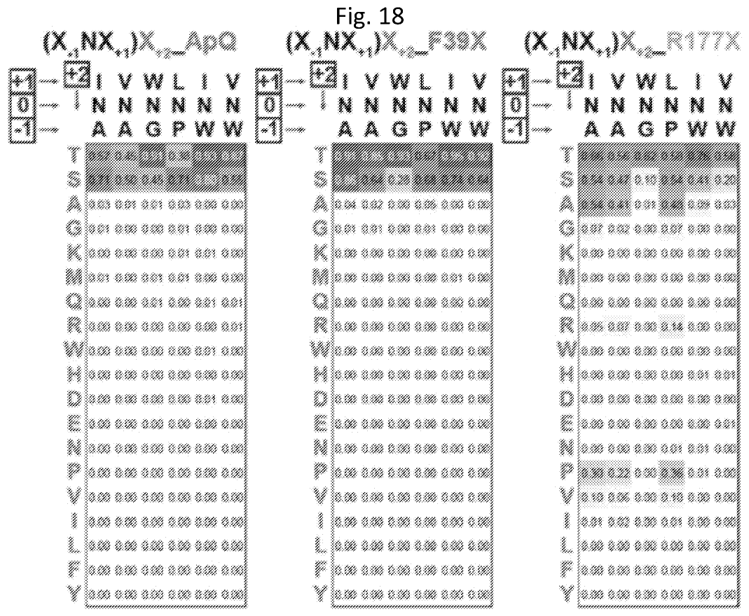

Next, in order to identify the residues that strongly influence specificity at each position of the substrate peptide, the inventors quantitatively compared the differences in substrate specificity for each of the SSVLs with that of ApQ. The inventors began by measuring the modification of the X₋₁-N-X₊₁-TRC peptide library at different concentrations of ApQ in order to generate a series of heatmaps for ApQ with various levels of average −ln(1−Y). In this way, the inventors could select the appropriate heatmap in order to compare the peptide selectivity difference of ApQ and each of the 26 protein SSVLs using heatmaps with the same value of average −ln(1−Y) (FIG. 16). The inventors then calculated the percentage difference of each X₋₁ amino acid (each row of the heatmap) for each SSVL compared to ApQ from the average −ln(1−Y) value for all 19 peptides within that X₋₁ amino acid, using the equation 2*|Ave(X)−Ave(ApQ)|/(Ave(X)+Ave (ApQ)), where Ave(X) and Ave(ApQ) are the average −ln(1-Y) for each SSVL and ApQ, respectively. The average of all 19 percentage differences in X₋₁ amino acid rows gave the mean percentage difference for the X₋₁ position (FIG. 1d). The inventors performed a similar analysis to determine the mean percentage difference for the X₊₁ position for each SSVL (FIG. 1d). The mean percentage difference heatmap of ApQ and all SSVLs compared to each other is shown in FIG. 17. Based on these data, the inventors concluded that the residues playing the strongest role in determining specificity of the enzyme for the X₋₁ position of the acceptor peptide are, in order from strongest to weakest: T438, A469, Y498, H214, and 1279. Similarly, the inventors found that, for the X₊₁ position, residues A469, H214, R177, H219, and T438 have the greatest impact on specificity. The inventors found that residue 469 plays a relatively strong role in determining enzyme specificity for both the X₋₁ and X₊₁ positions, as well as the UDP sugar donor as reported previously^{29, 30}.

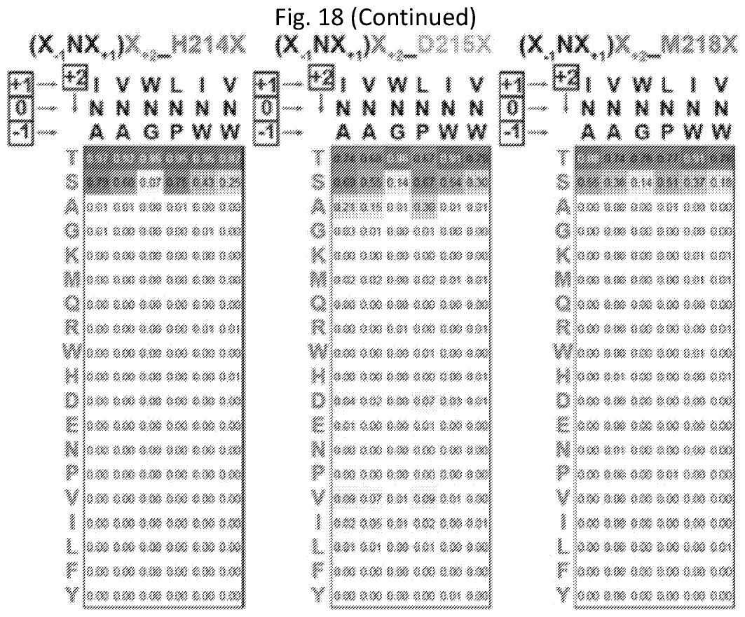

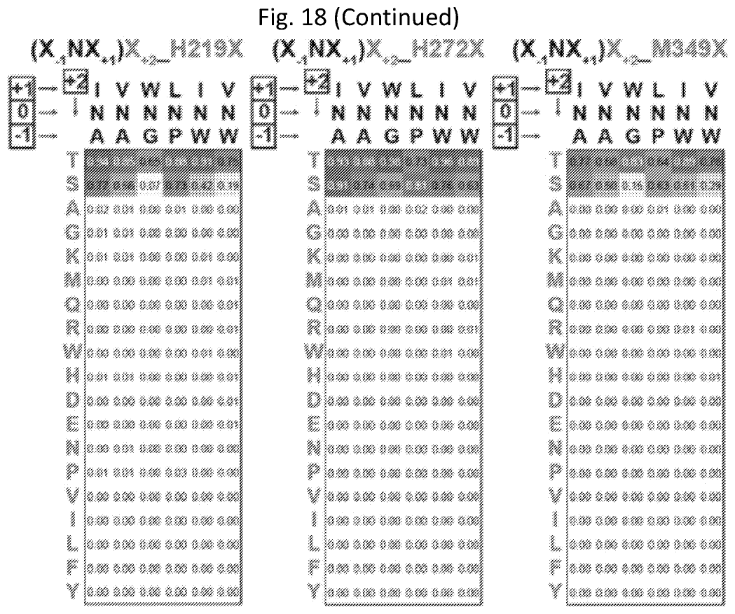

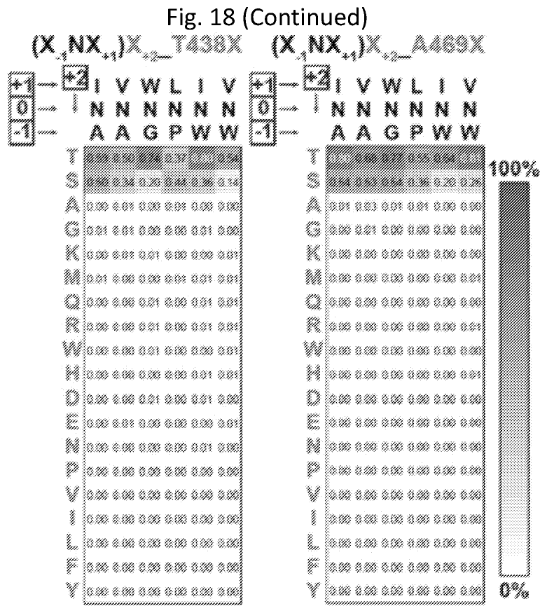

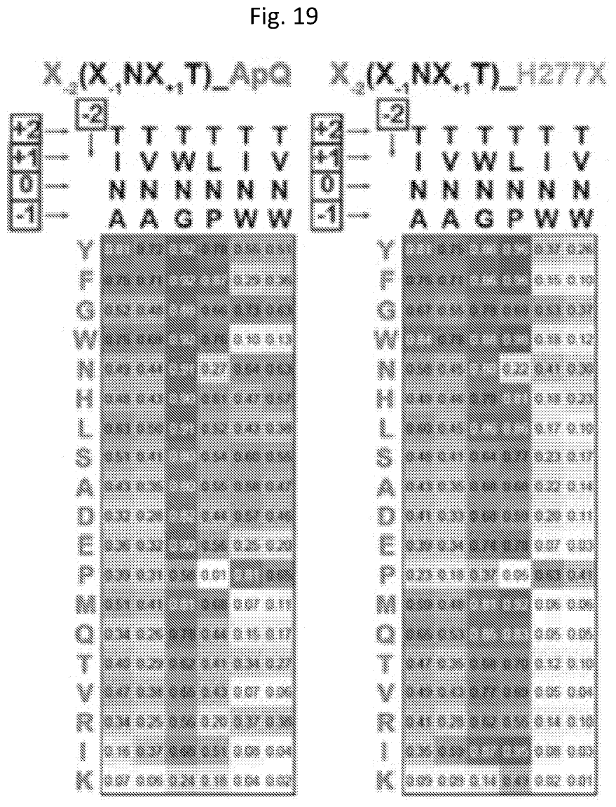

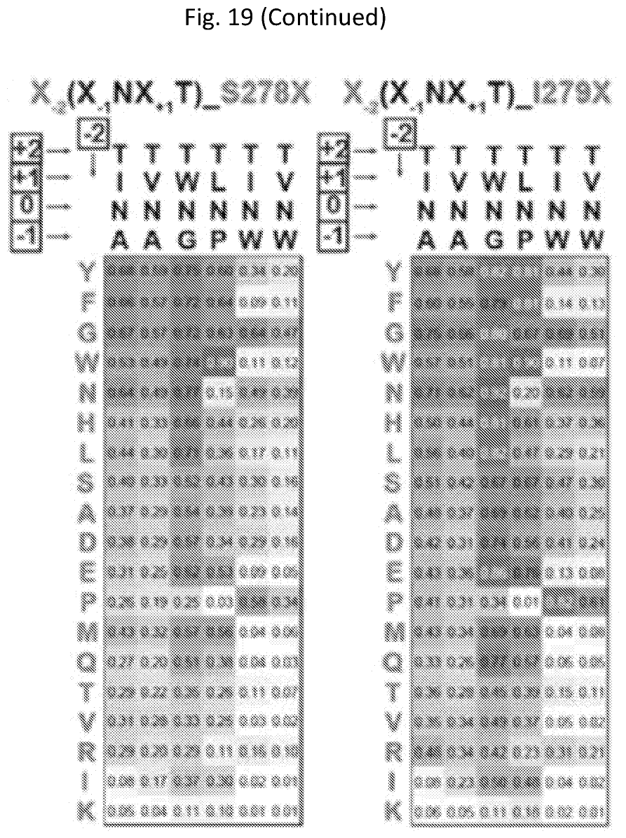

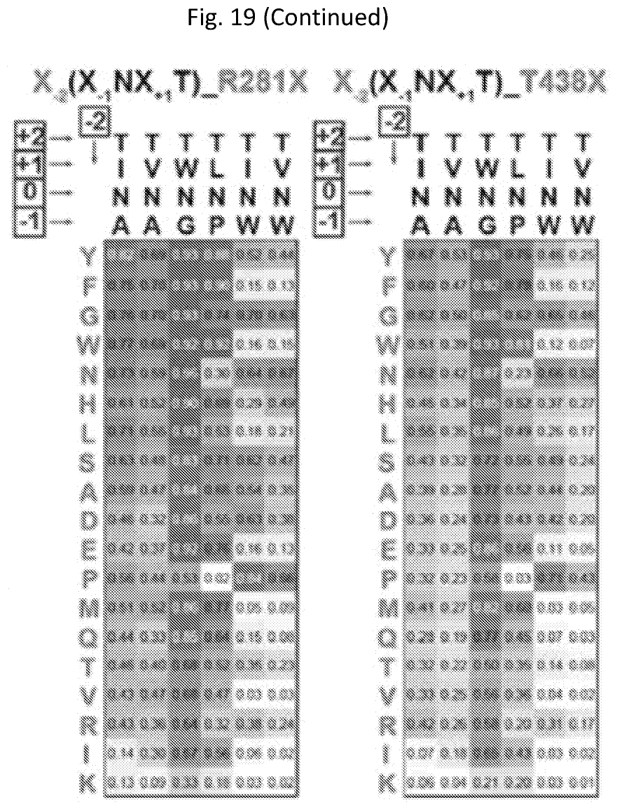

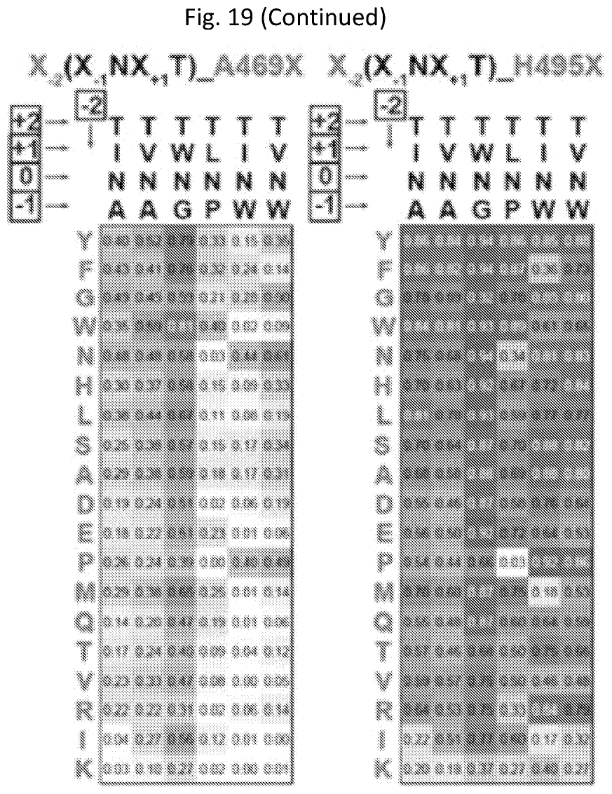

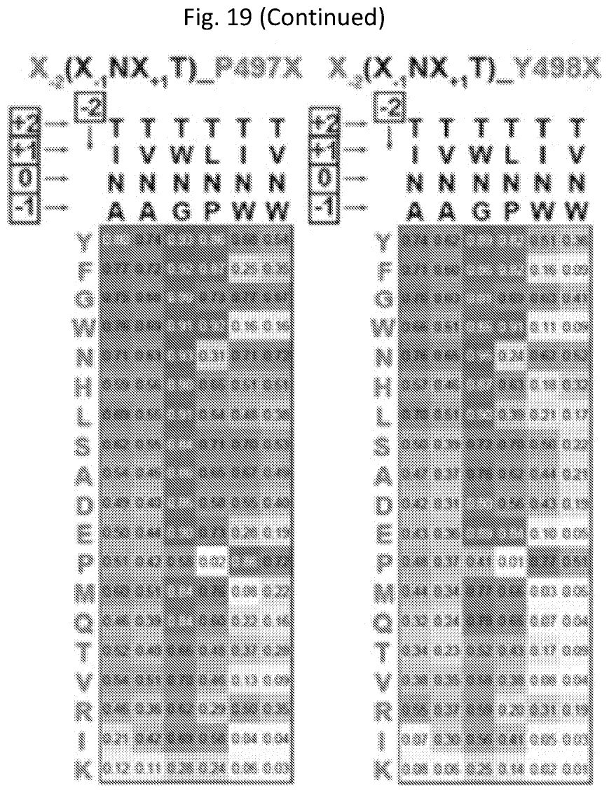

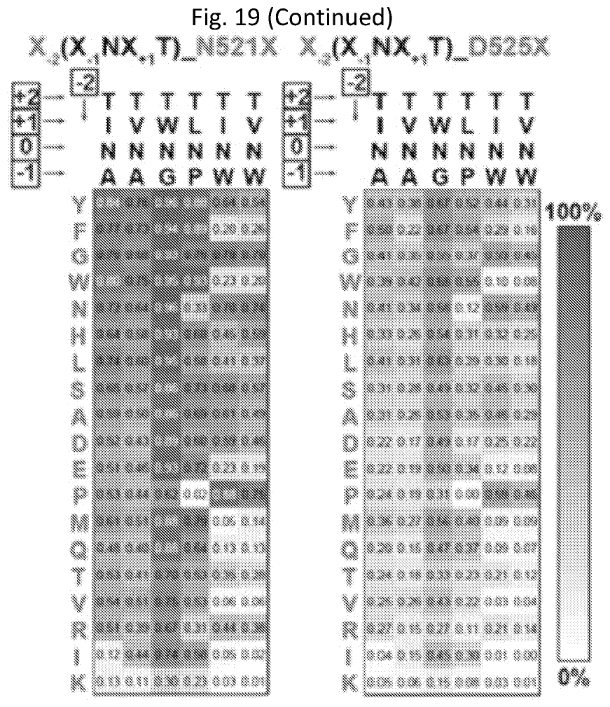

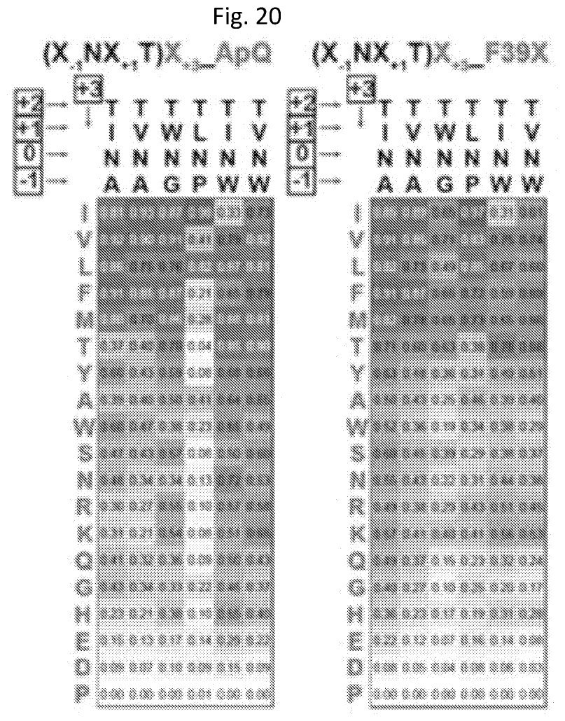

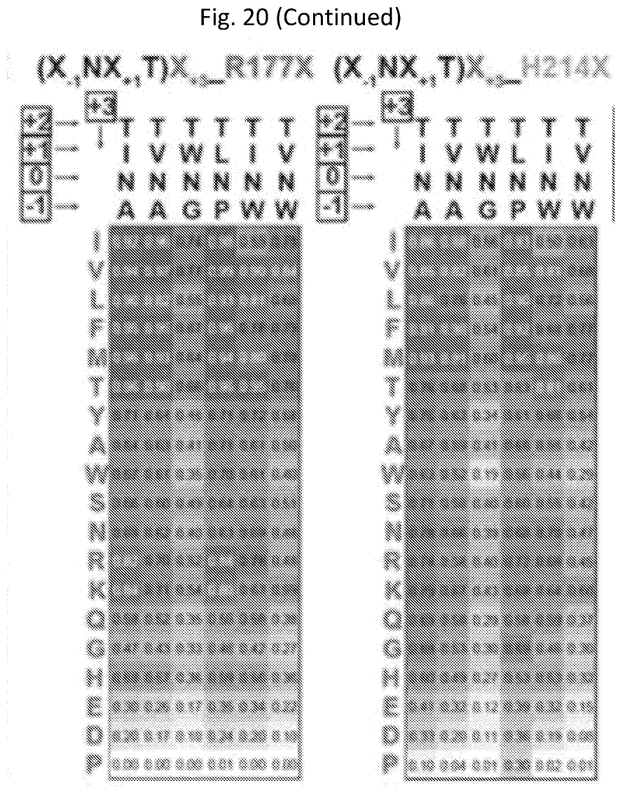

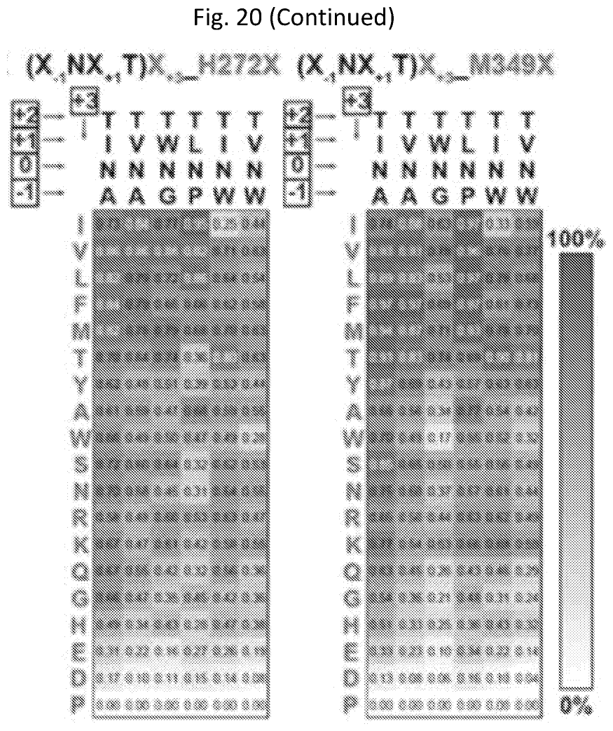

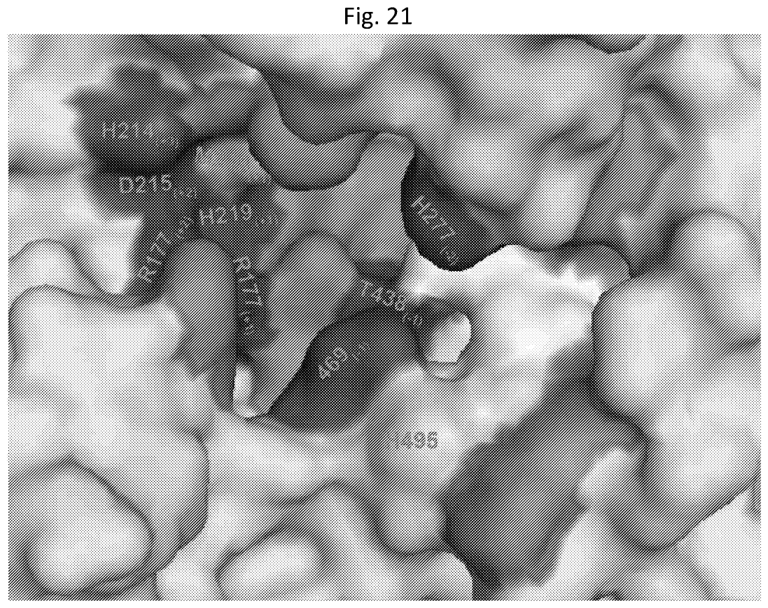

Having identified the residues that play the strongest roles in determining the polypeptide specificity of ApQ for the X₋₁ and X₊₁ positions of the acceptor peptide, the inventors next pursued analogous experiments to identify residues that affect specificity for the X₊₂, X₋₂, and X₊₃ positions. Here, the inventors chose six representative X₋₁-N-X₊₁-TRC peptide sequences preferred by ApQ and produced new peptide libraries that substituted the Thr with the other 18 amino acids at the X₊₂ position, adding 19 X₋₂ amino acids, or inserting 19 X₊₃ amino acids (again, with Cys excluded in all peptides), respectively. The inventors then screened the specificity of select SSVLs for each position that the earlier experiments suggested to have an important role in specificity. The inventors found that D215 and R177 were important in determining specificity for the X₊₂ position, H277 for X₋₂, and H214 for X₊₃ (FIGS. 18-20). These observations identify those residues that interact with and determine specificity for the acceptor peptide (shown in FIG. 21); however, they do not rigorously establish that the interactions are direct.

2. Screening Individual NGT Mutants with Unique Substrate Specificities

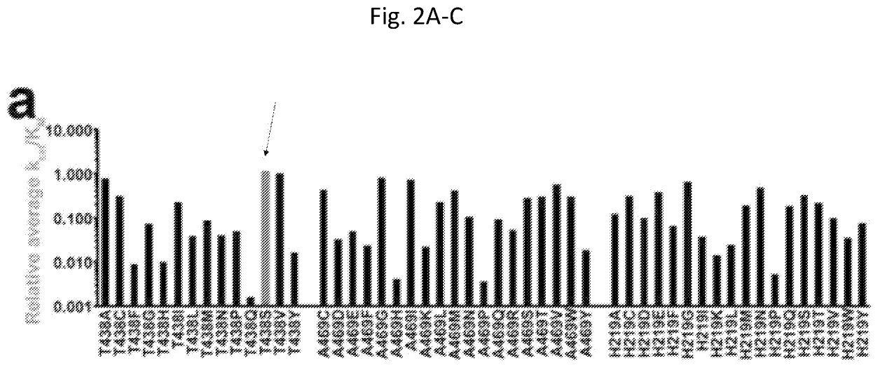

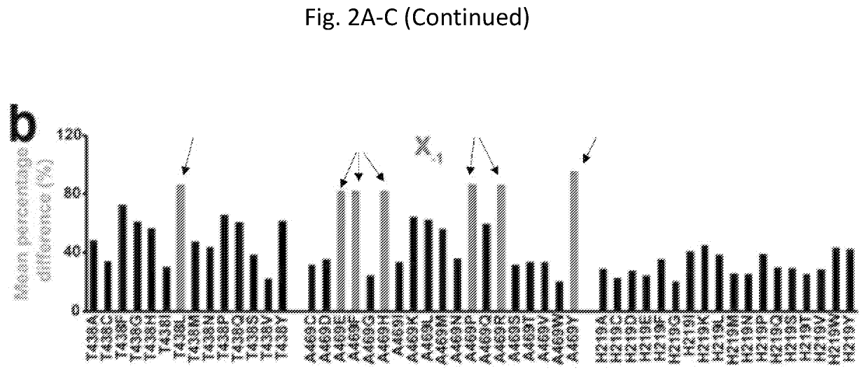

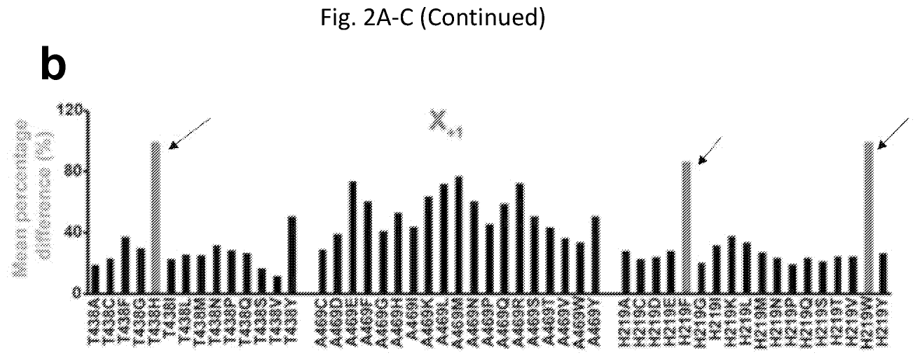

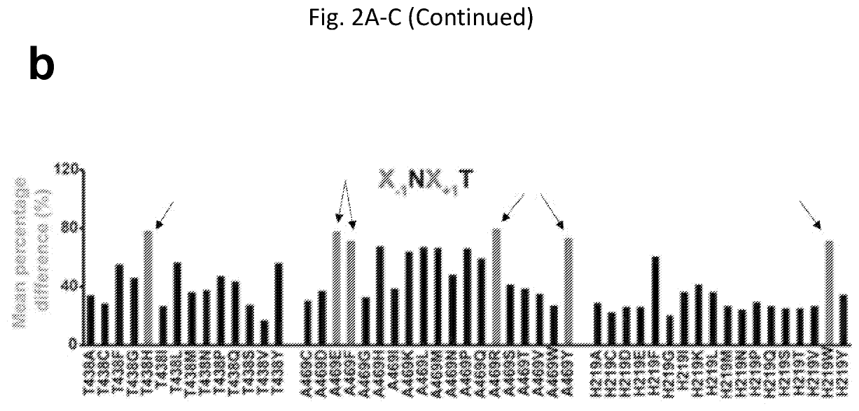

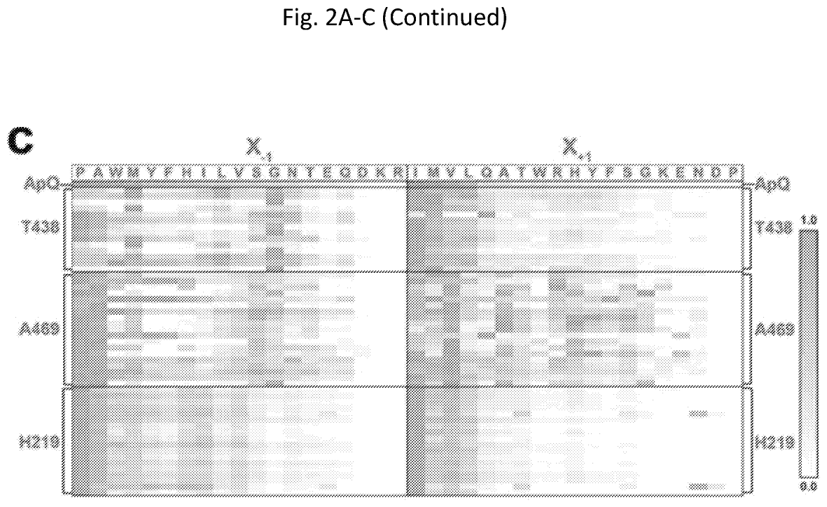

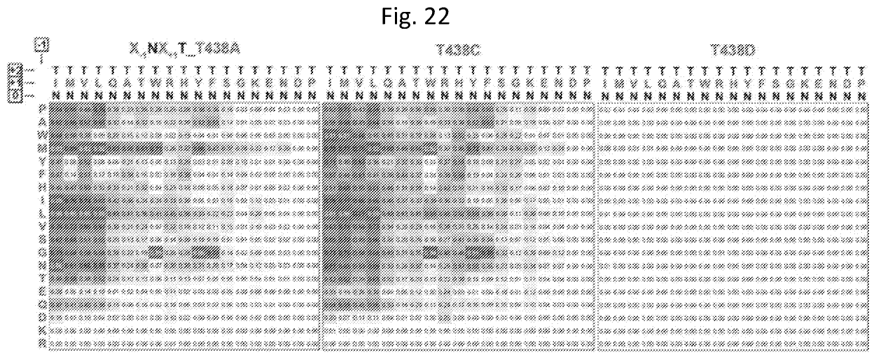

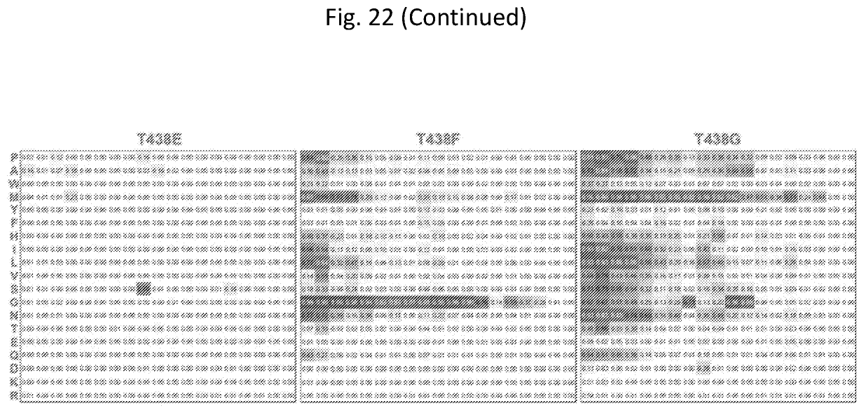

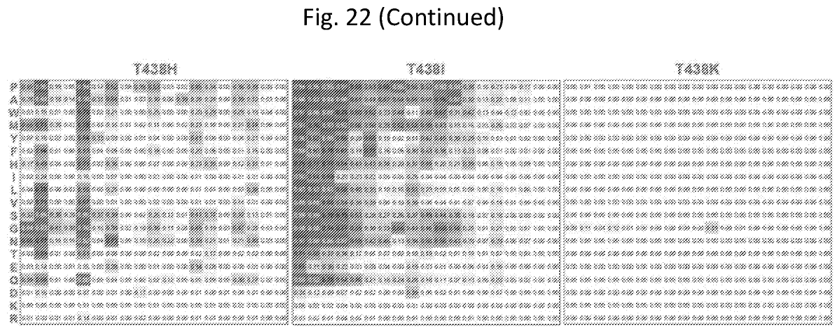

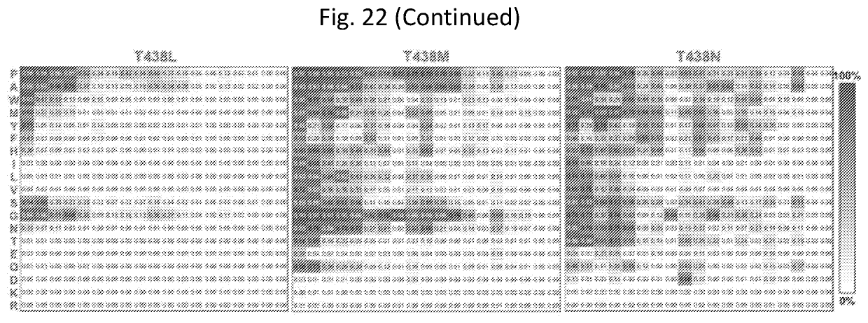

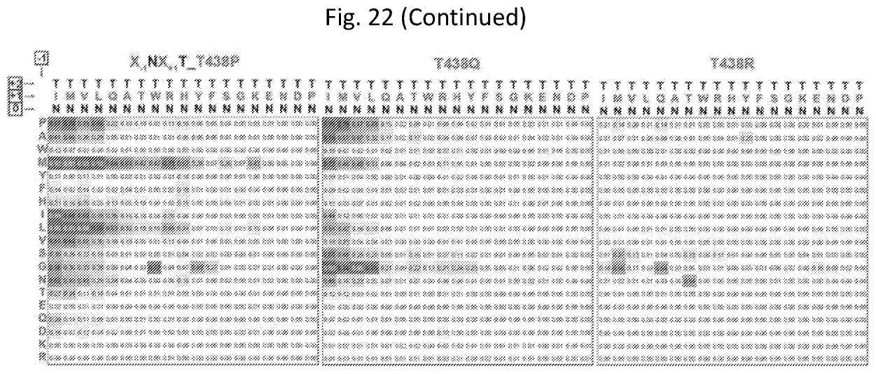

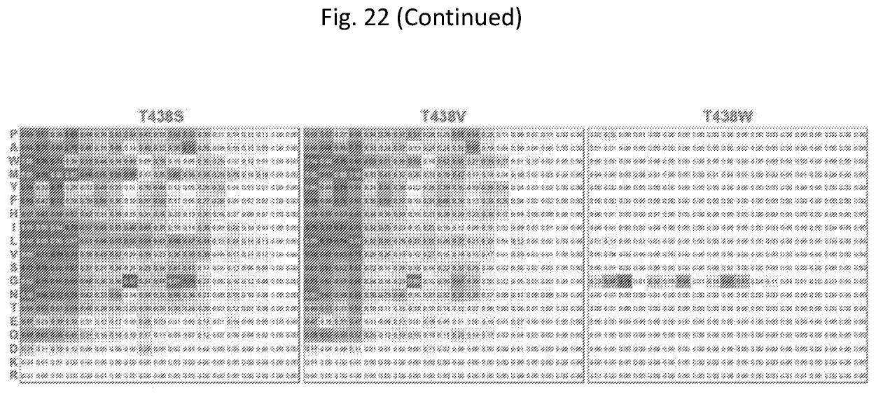

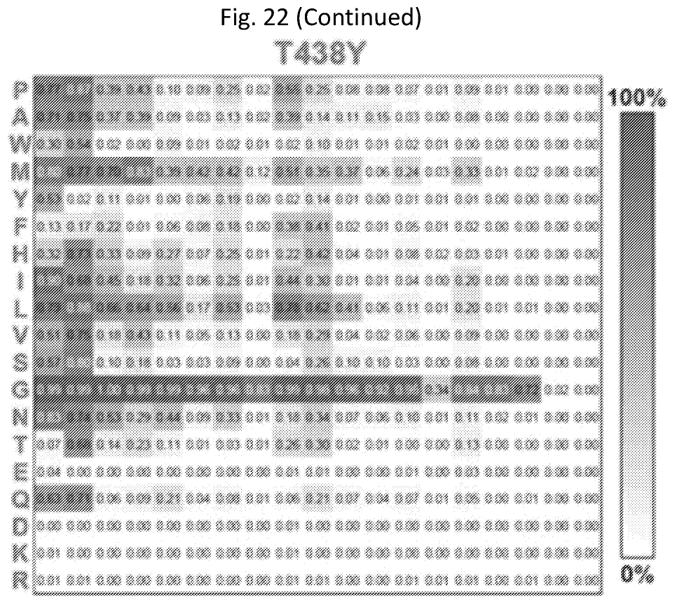

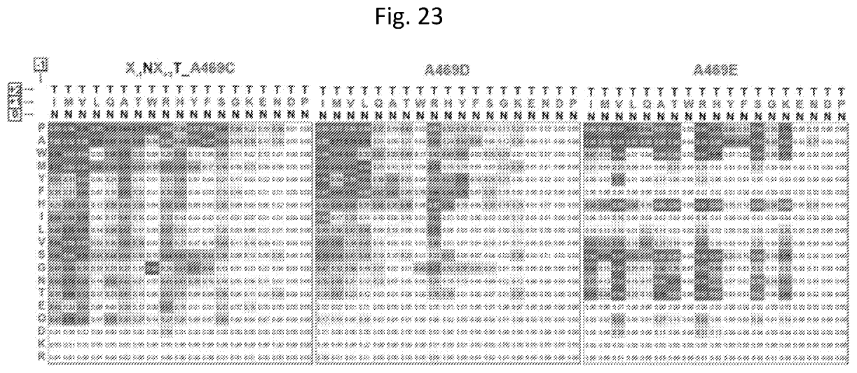

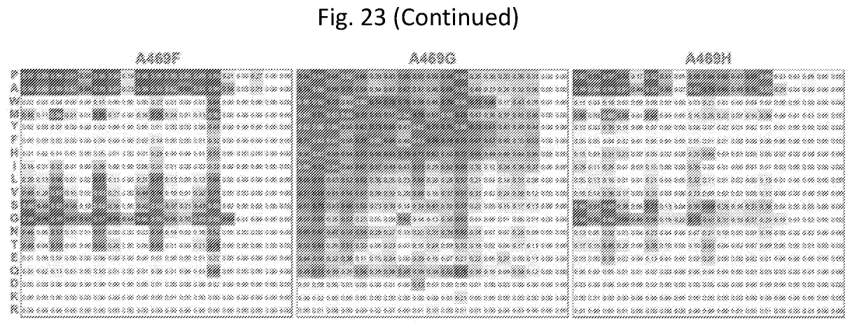

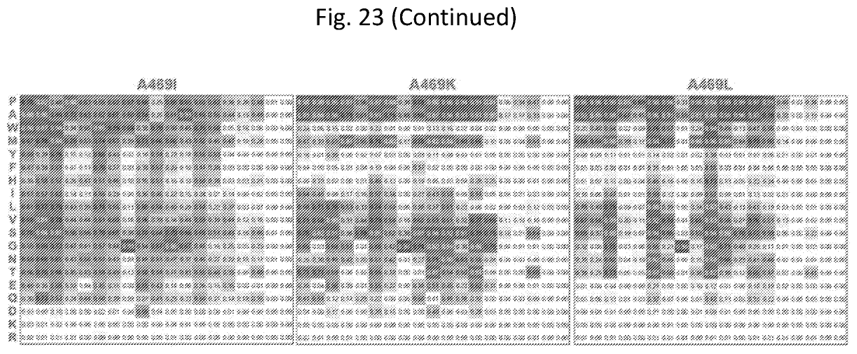

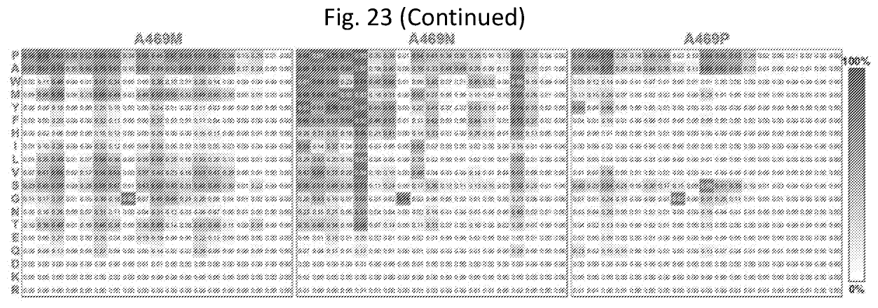

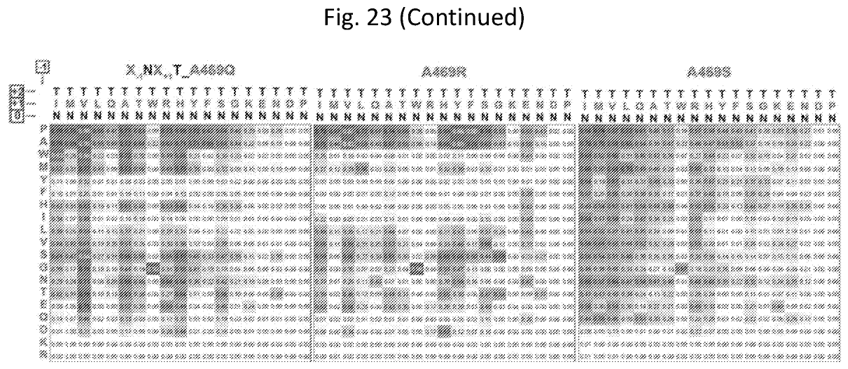

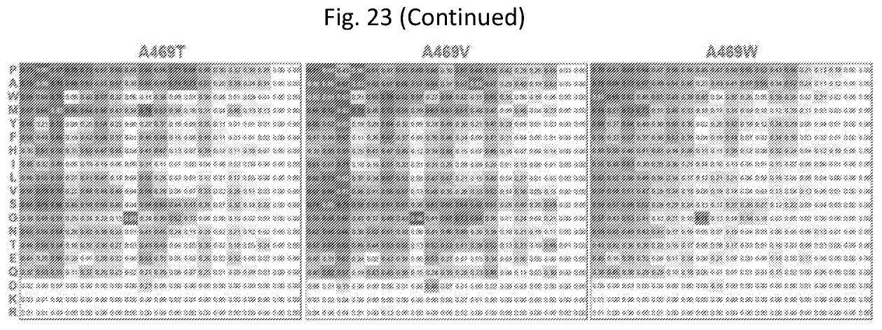

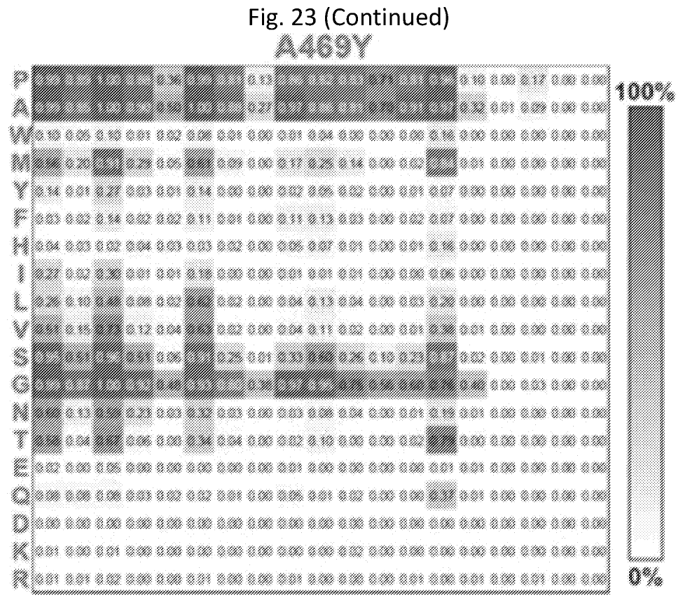

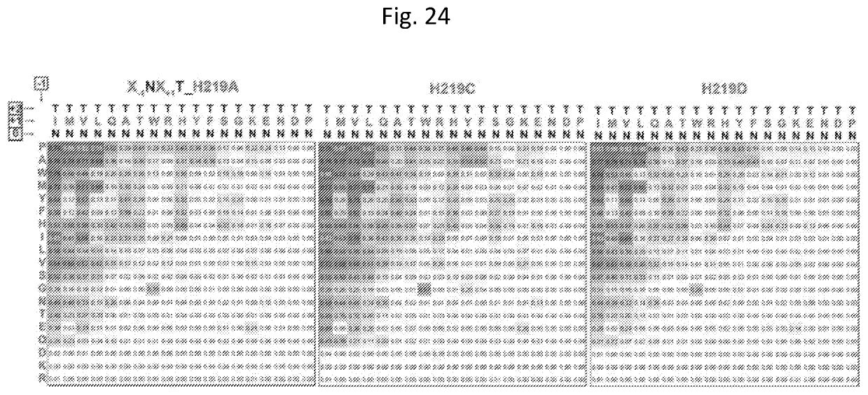

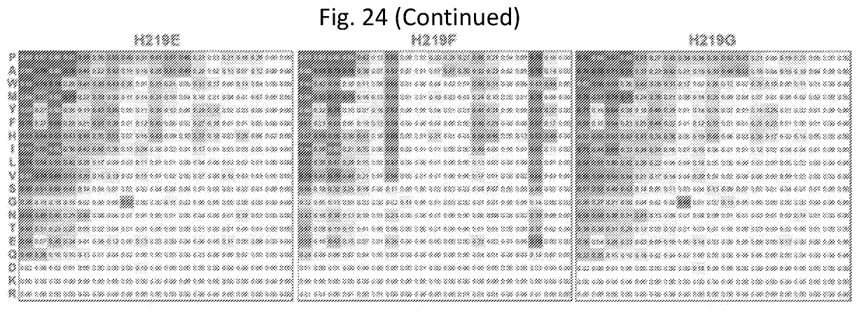

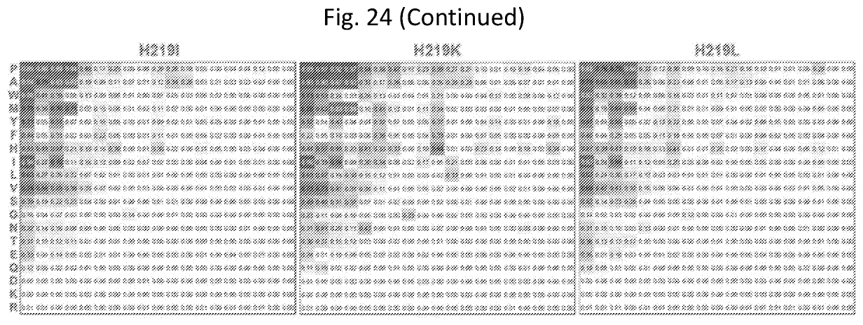

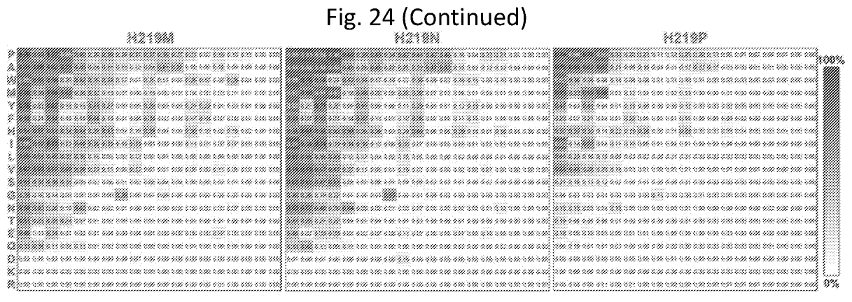

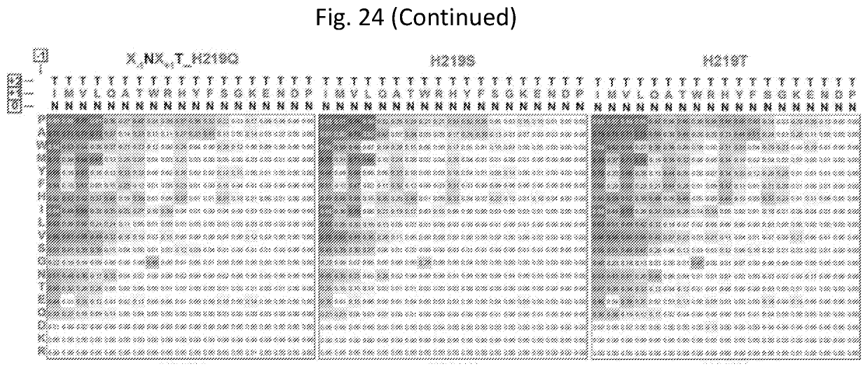

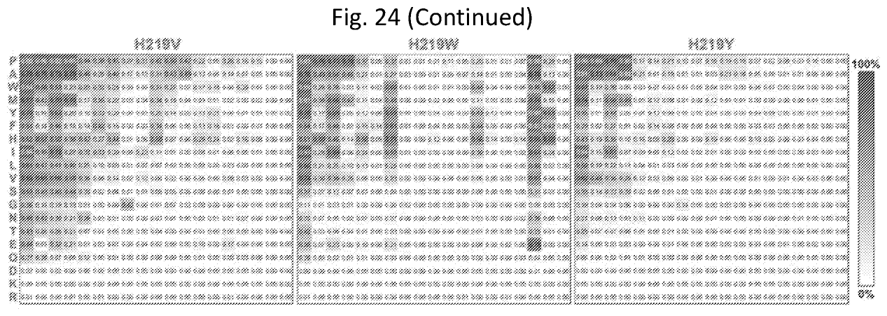

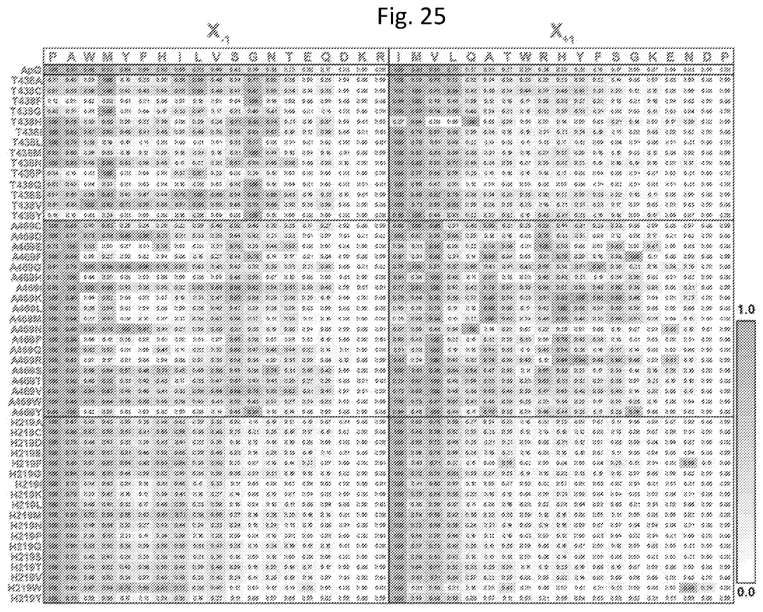

After identifying the specificity-determining residues, the inventors sought to screen the individual mutants at these residues to understand which peptide sequences were preferred as substrates. Based on their analyses, the inventors decided to deconvolute the activities of each mutant within three of the 26 SSVLs-H219, T438, and A469-using the X₋₁-N-X₊₁-TRC peptide library (FIGS. 22-24). The inventors first isolated individual mutants from the SSVLs by circularization of the linear DNA and transformation of the resulting plasmids (see Methods). The inventors found that each individual variant was expressed at similar levels (FIG. 14A-E). Only T438S showed an increase in average glycosylation activity over ApQ (FIG. 2a), while T438D/E/K/R/W and H219R had the poorest activity (less than 0.1% relative to ApQ). The activities of mutants at T438, likely an important residue for peptide binding according to the screen, varied significantly over the 19 mutants. The inventors also analyzed the peptide selectivity for these individual mutants (FIG. 2b). Most of T438 mutants exhibited altered specificities for the X₋₁ position, with little effect on the X₊₁ position; however, T438H showed altered X₊₁ specificity and small changes in X₋₁ specificity. Most A469 mutants showed different preferences for both the X₋₁ and X₊₁ positions. Mutations of A469 to F/H/P/R/Y had a stronger effect on X₋₁ specificity while to G/I/N/S had a stronger effect on X₊₁. Of the H219 mutants, only H219F/W strongly affected the peptide selectivity at the X₊₁ position.

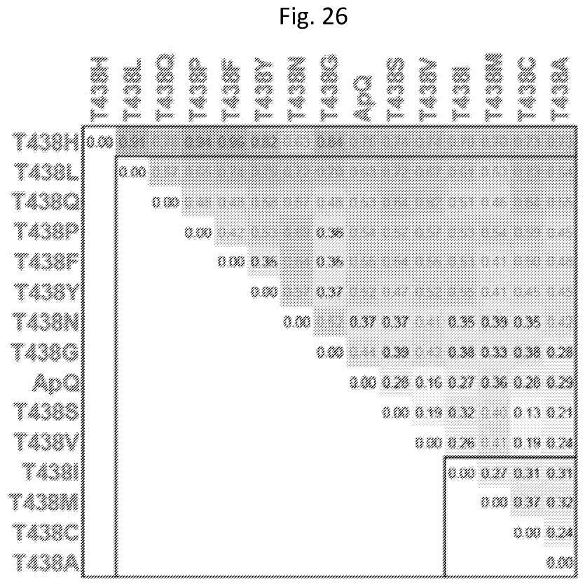

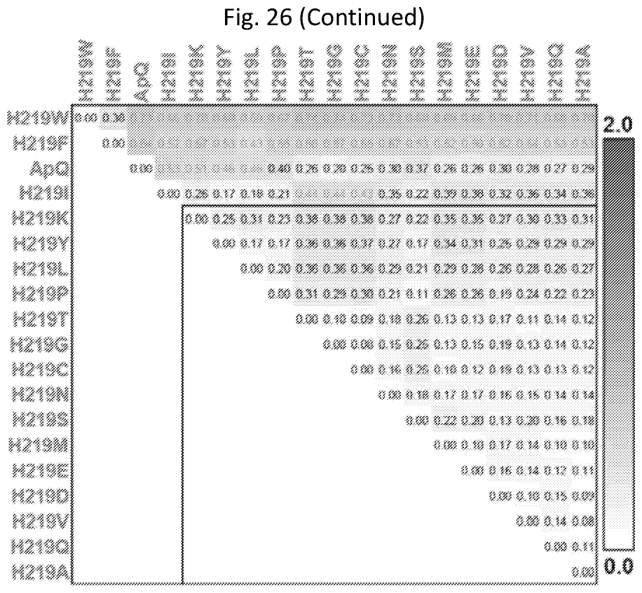

The differential selectivity of each mutant on amino acids at the X₋₁ or X₊₁ position allows them to be used for unique purposes (e.g., site-specific modification) (FIG. 2c). For example, most T438 mutants preferred other amino acids over Pro and Ala at X₋₁ while most A469 mutants preferred other amino acids over Ile and Met at X₊₁; H219F and H219W had very similar peptide specificities and exhibited significant increases in their preference for peptides with Asn and Asp at X₊₁. The inventors also performed a pairwise comparison of the specificity differences between all individual NGT mutants at each residue and found that many mutants possess unique preferences (FIG. 26).

### 3. Expanding the Set of Sequences Eligible of Glycosylation by Selected NGT Mutants

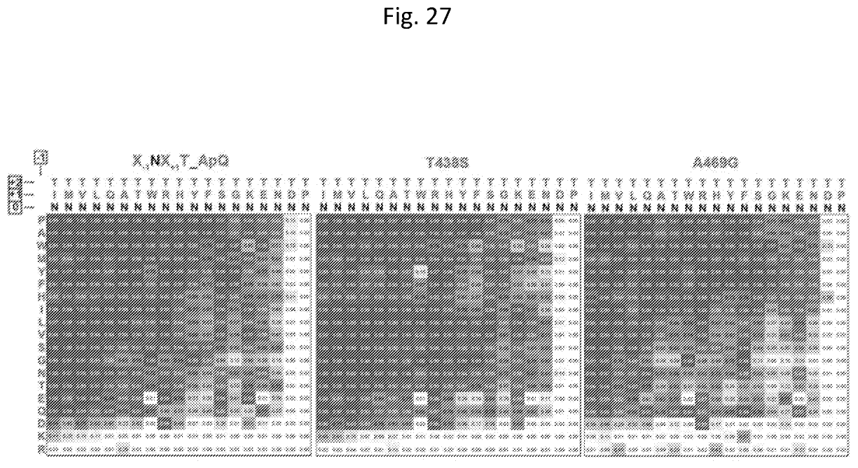

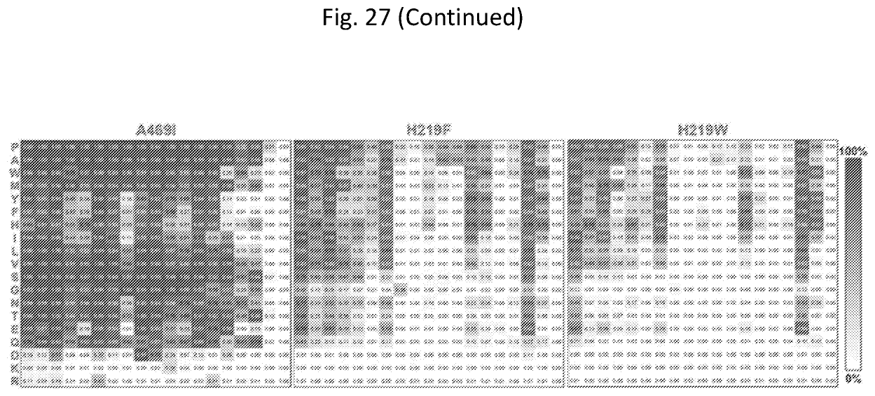

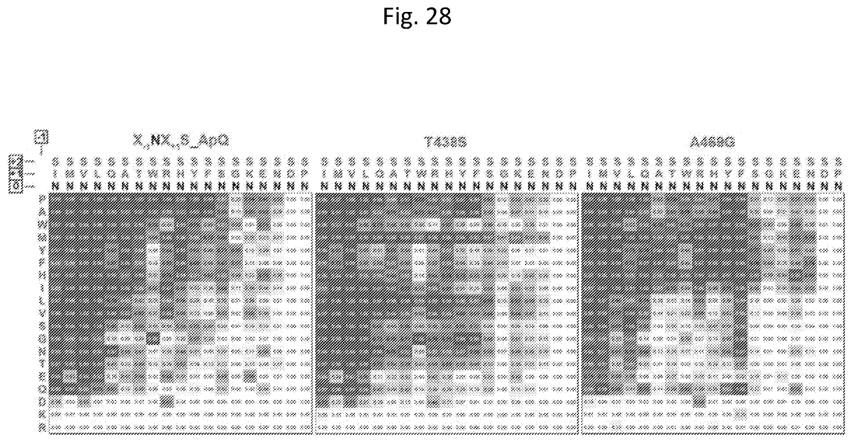

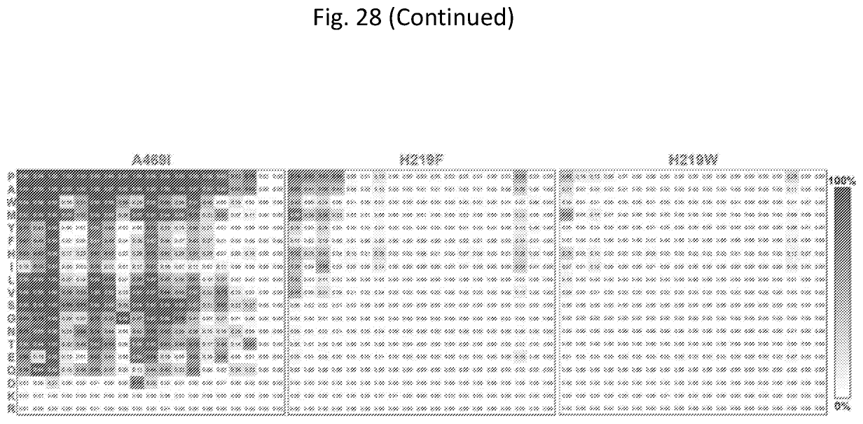

To identify a panel of mutant NGTs that can modify comprehensive sets of acceptor sequences, the inventors first selected six NGTs (T438S, A469G, A469I, H219F and H219W, as well as ApQ) arising from their initial screens. This panel of NGTs combined to provide the highest activity for the broadest range of peptides in the initial X₋₁-N-X₊₁-TRC substrate library (based on the calculated appropriate k_{cat}/K_{M} for each peptide-NGT combination). The inventors then screened the activities of these NGTs under identical conditions (0.545 μM NGT for 3 h at 30° C.) across a total of 684 peptide sequences of the form X₋₁-N-X₊₁-TRC and X₋₁-N-X₊₁-SRC (FIG. 27-28). These six NGTs all displayed less activity with Ser than Thr at the X₊₂ position. The five ApQ mutants added to the panel significantly expanded the set of sequences within the X₋₁-N-X₊₁-S/T motif that can be efficiently glycosylated (where the modification was greater than 80%) by 17% (118 of 684 peptides).

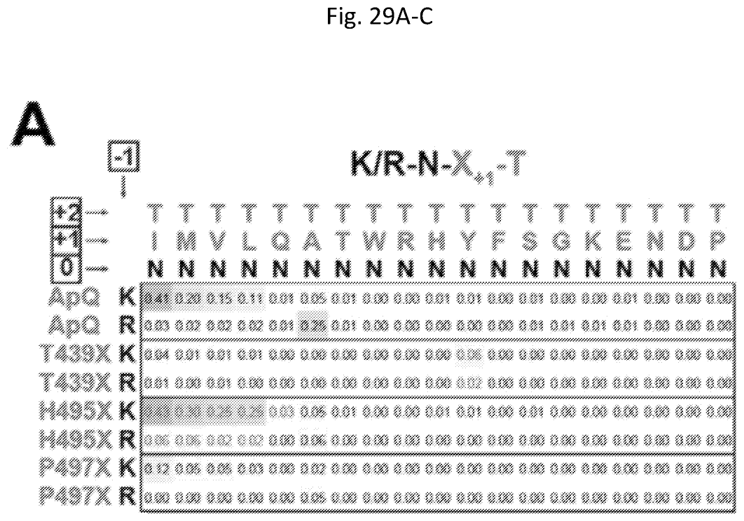

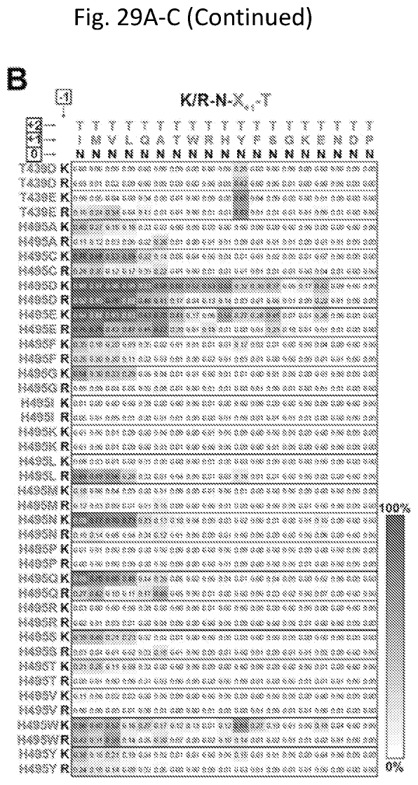

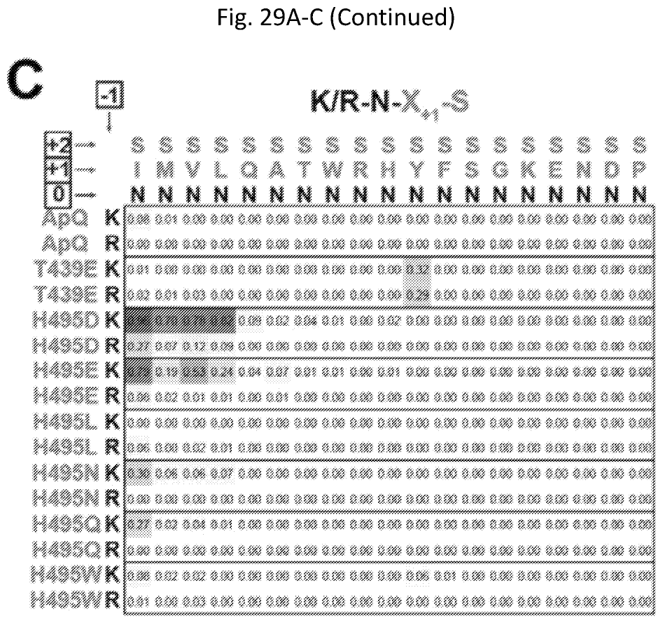

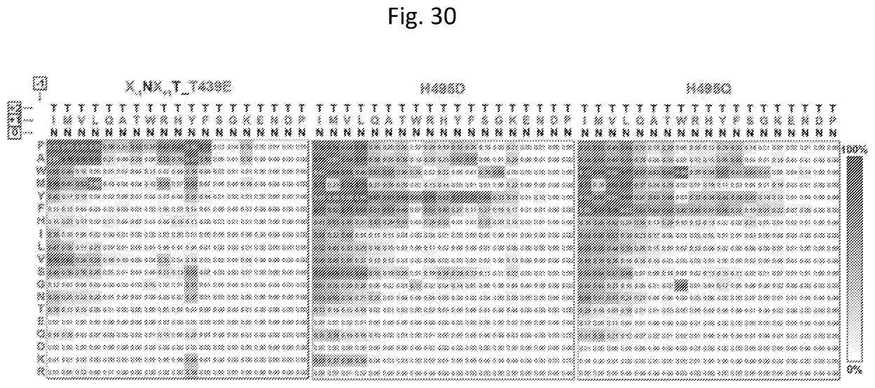

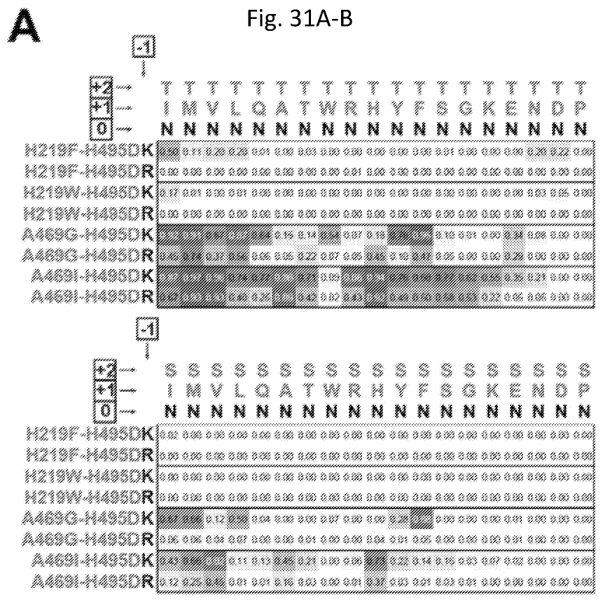

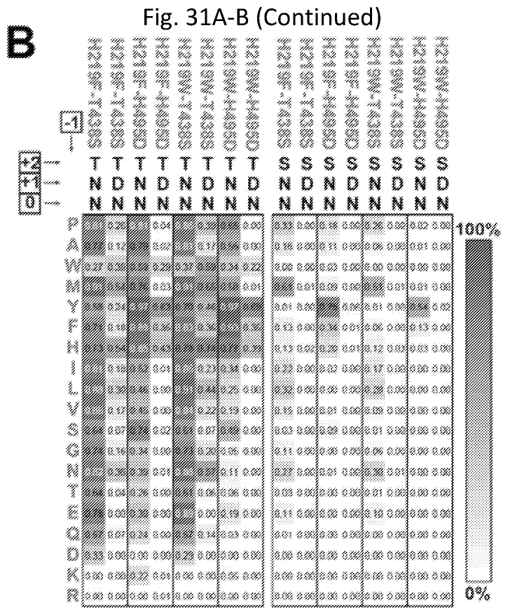

However, the inventors noticed that even with these five additional ApQ mutants, the glycosylation of peptide substrates with Lys or Arg at the X₋₁ position remained challenging. To address this gap and further expand the permissible substrate scope, the inventors tested enzymes with mutations to residues that are nearby the hypothesized X₋₁ binding site (FIG. 21) and exhibited high activity in SSVL screens—T439, H495, and P497 (details described in FIG. 29A-C). The inventors found that T439E and some individual H495 mutants (especially H495D) showed significantly increased preferences for peptides with Lys or Arg at the X₋₁ position (heatmaps in modification of the full X₋₁-N-X₊₁-TRC substrate library by representative mutants are shown in FIG. 30). To further expand the set of preferred sequences, the inventors also generated and screened double mutants that combined two single mutations identified above. Specifically, the inventors combined H495D with mutations that provided unique specificities at the X₊₁ position (A469G, A469I, and H219F/W). The inventors also combined the H219F/W with mutations that provided unique specificities at X₋₁ (T438S and H495D) (FIG. 31A-B).

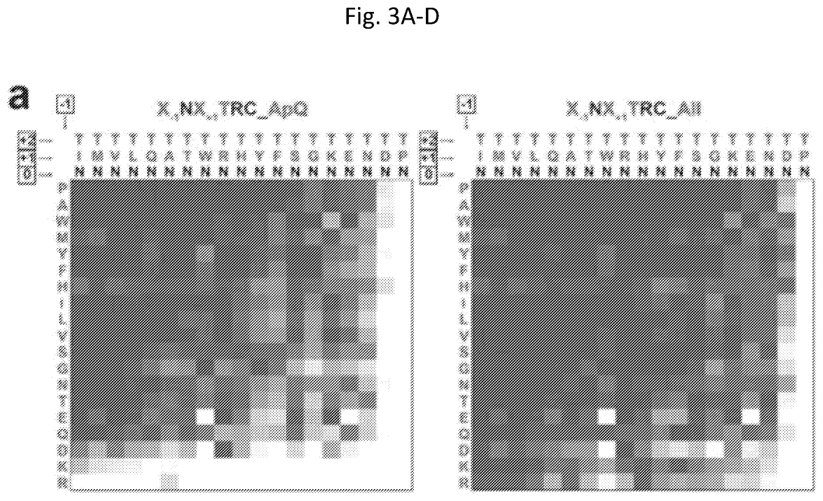

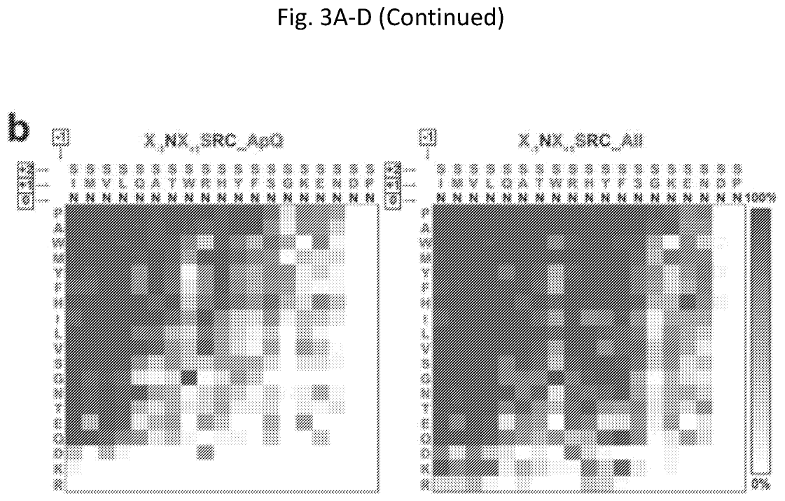

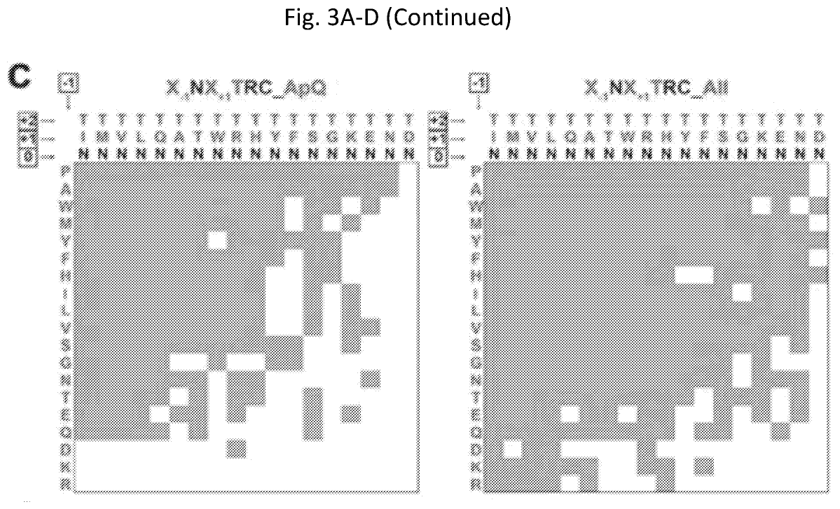

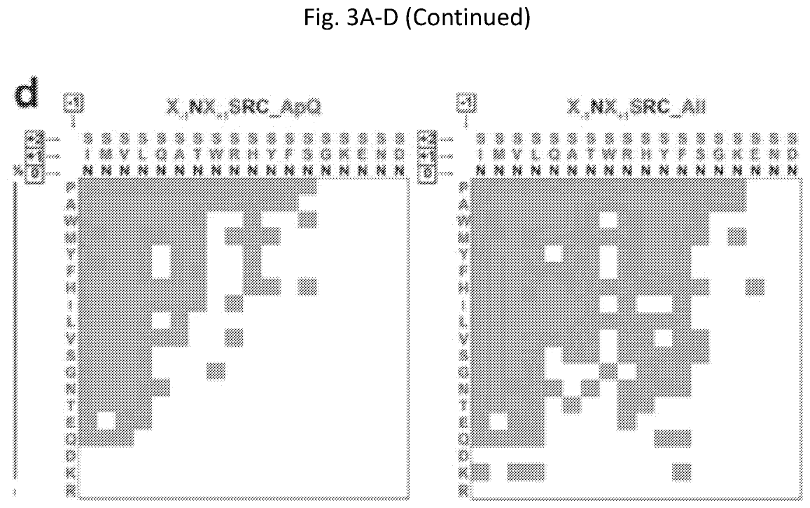

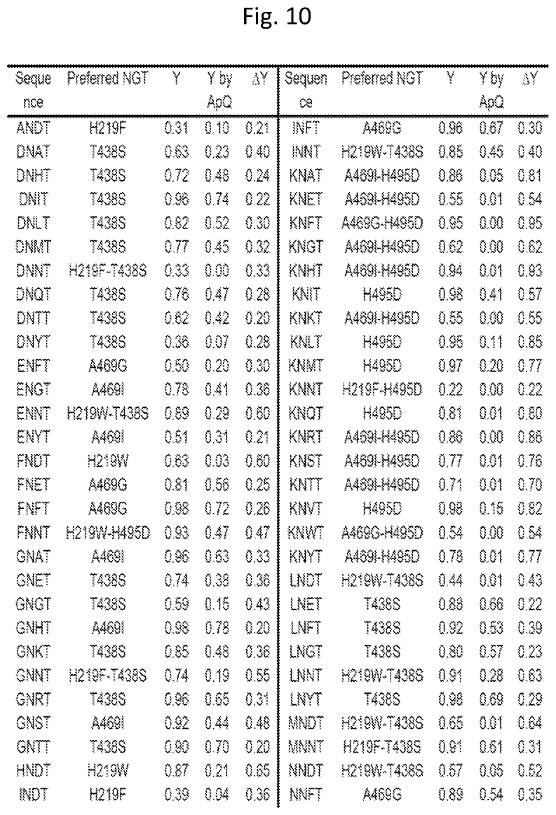

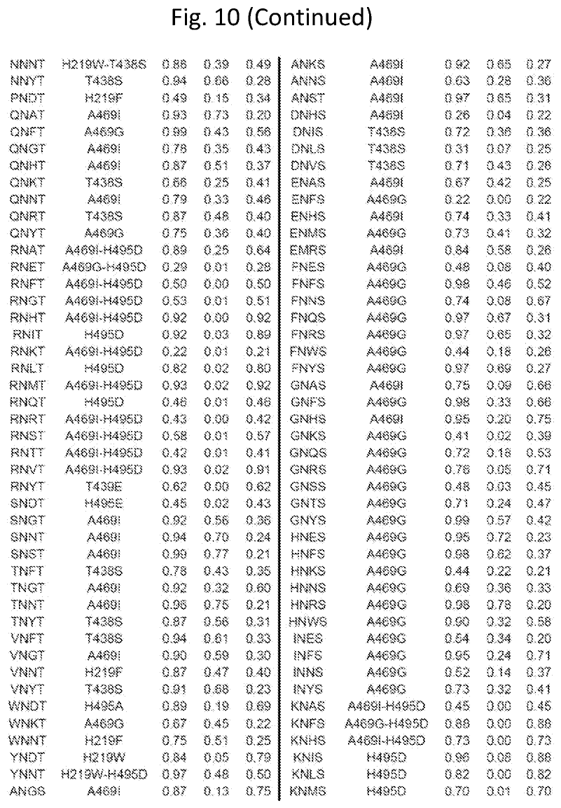

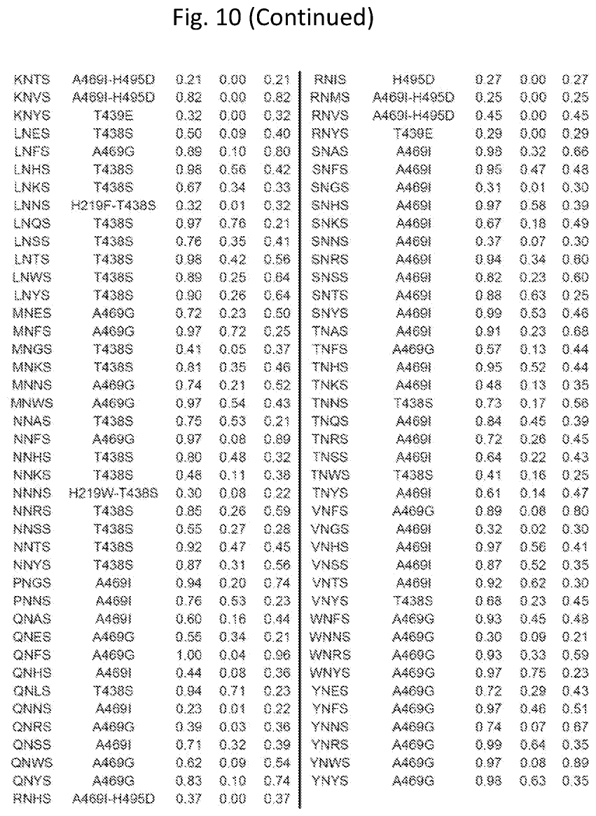

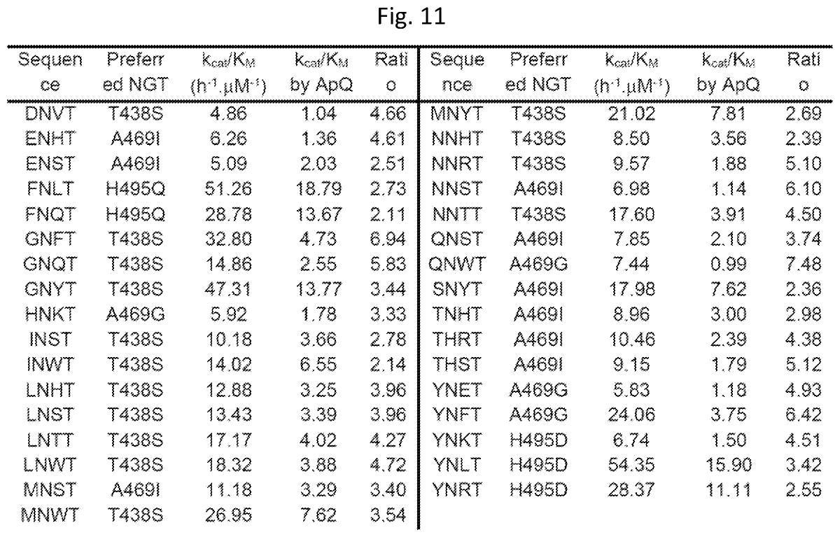

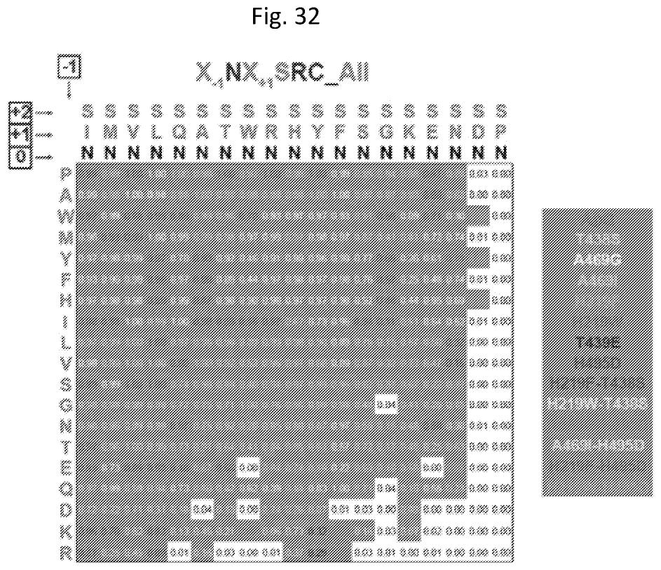

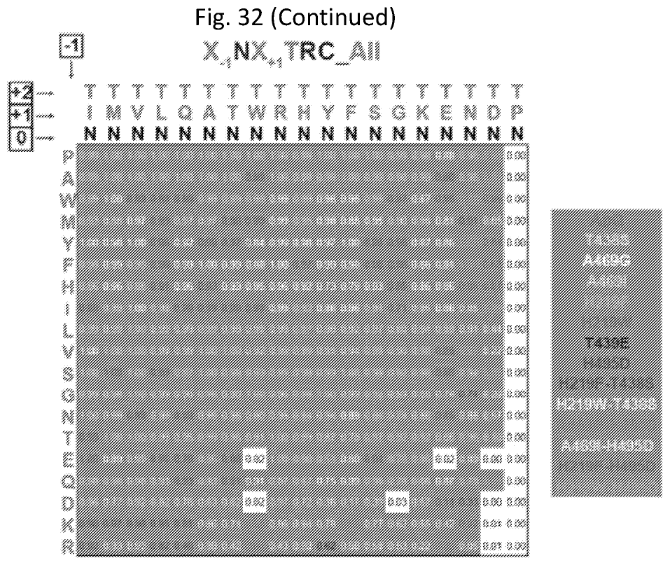

Finally, the inventors assembled and tested a panel of 14 selected NGTs (ApQ, H219F, H219W, T438S, T439E, A469G, A469I, H495D, H219F-T438S, H219F-H495D, H219W-T438S, H219W-H495D, A469G-H495D, and A469I-H495D) with the entire or partial X₋₁-N-X₊₁-TRC and X₋₁-N-X₊₁-SRC peptide libraries under identical reaction conditions (FIG. 27-29, 31). Their goal was to demonstrate that this NGT panel could enable the glycosylation of a diverse range of peptides. The inventors observed that these enzymes did significantly increase the maximum modification efficiency for 260 of the 684 canonical X₋₁-N-X₊₁-S/T glycosylation sequences over ApQ alone (FIG. 3a-b and FIGS. 10-11). Specifically, the inventors increased the percentage of peptides with modification greater than 80% from the 45% to 66% (FIG. 3c-d) and the percentage of peptides with modification greater than 5% from 80% to 93% (FIG. 32).

### 4. Glycosylation of Approved Therapeutic Proteins Using NGT Mutants

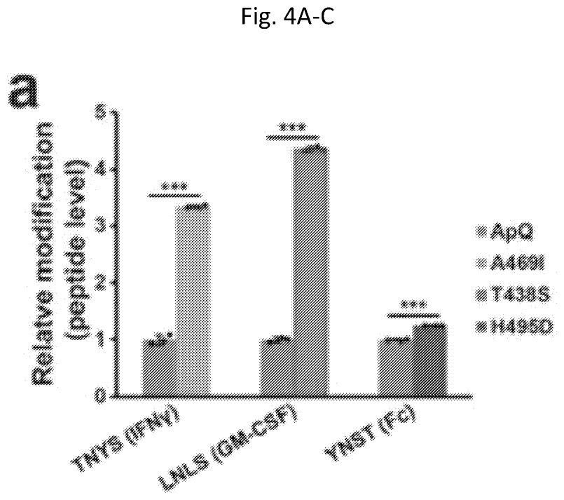

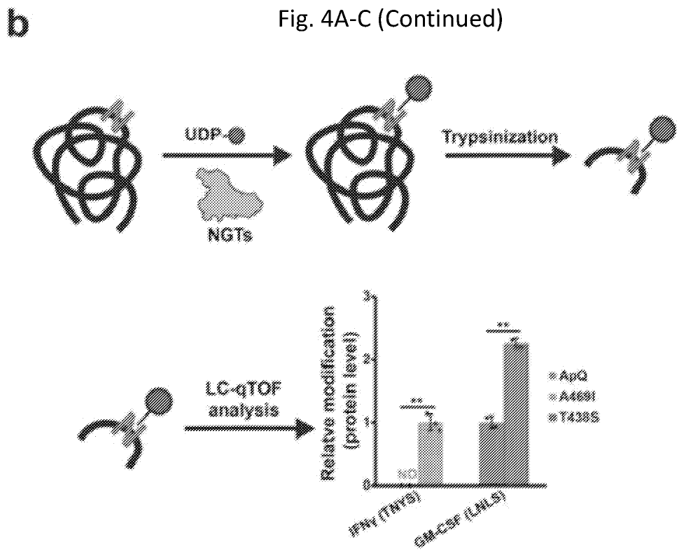

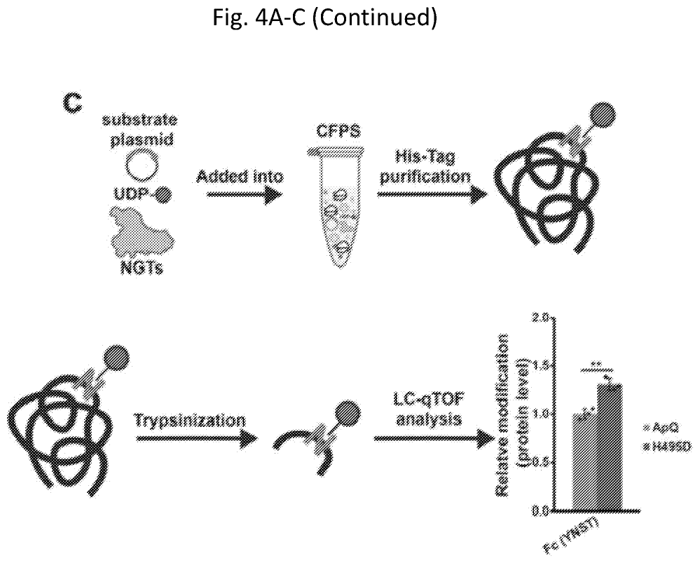

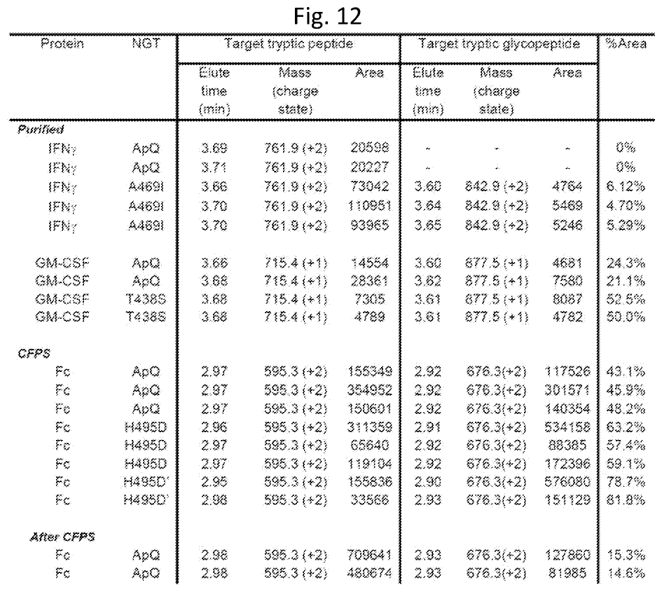

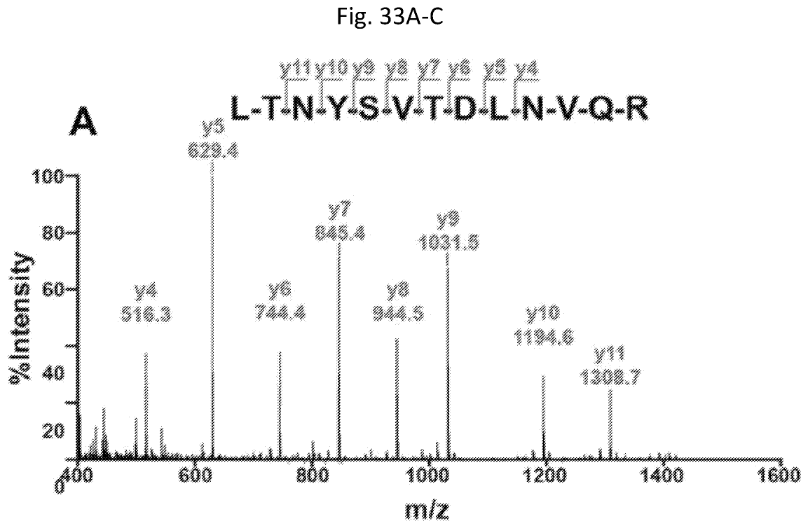

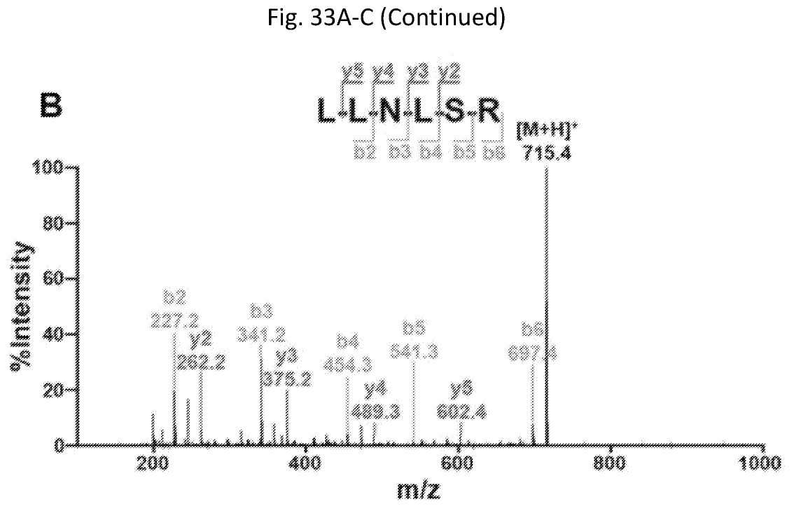

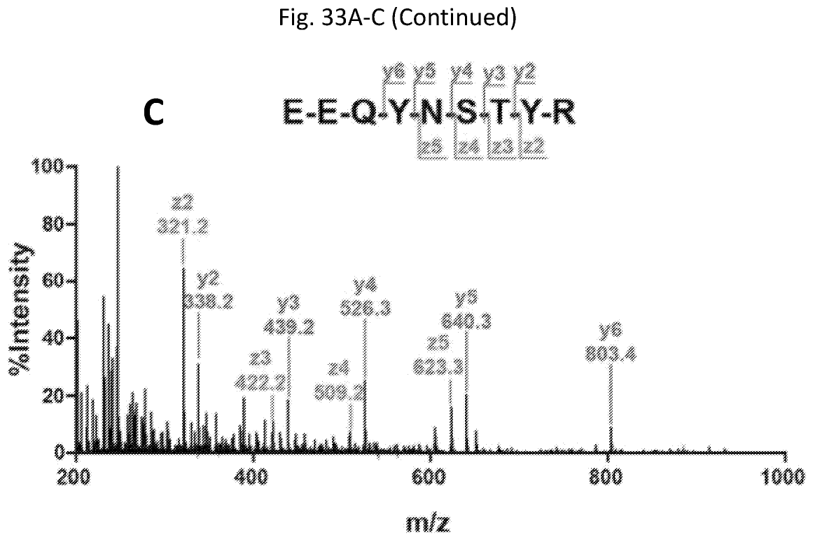

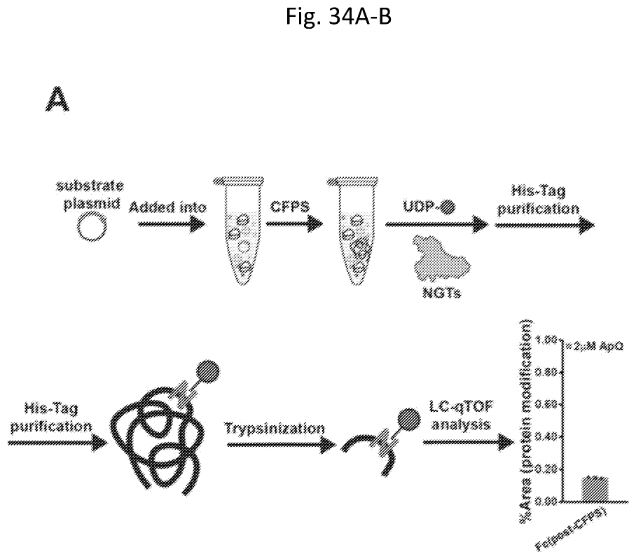

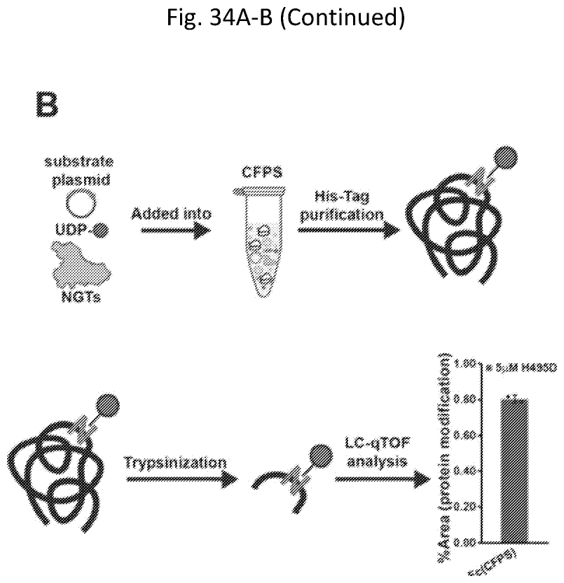

As a proof of principle, the inventors next demonstrated the utility of specific mutants from the NGT mutant panel developed above to glycosylate model therapeutic proteins: Interferon-gamma (IFNγ), Granulocyte-Macrophage Colony-Stimulating Factor (GM-CSF), and the constant region (Fc) of a human immunoglobulin antibody (IgG1). At the peptide level, the inventors found that the purified A469I mutant glycosylated the sequence TNYS found in IFNγ more efficiently than did ApQ (FIG. 4a). Similarly, T438S glycosylated the LNLS sequence from GM-CSF more efficiently than did ApQ, and H495D glycosylated the YNST sequence from Fc more efficiently than did ApQ (FIG. 4a). The inventors then confirmed these relative activities at the protein level using purified IFNγ and GM-CSF as substrates. After glycosylation, the target protein was digested with trypsin and analyzed by liquid chromatography-quadrupole time-of-flight (LC-qTOF) mass spectrometry (FIG. 4b). The relative modification, using the % area of integrated extracted ion chromatograms, showed that the NGT mutant enabled more efficient glycosylation than did ApQ (FIG. 4b). The inventors also used MS² to confirm the identity of the targeted peptides (FIG. 33A-C). Notably, the glycosylation of sequences within folded intact proteins is less efficient than that of those sequences as free peptides. Using Fc as an example, the inventors showed that the modification of proteins could be improved by supplementing NGTs at the beginning of the CFPS reaction to simultaneously express and glycosylate substrate proteins. By adding 2 μM ApQ before rather than after the Fc expression in the CFPS reaction, the inventors found that the glycosylation efficiency of Fc was increased from 15% to 46% (FIG. 34A-B and FIG. 12). Supplementing 2 μM H495D mutant rather than ApQ pre-CFPS significantly increased the modification of Fc to 60% or by 1.3-fold (FIG. 4c and FIG. 12). By supplementing with 5 μM of H495D, 80% modification of Fc could be achieved (FIG. 34A-B). Taken together, these data show that native amino acids sequences with canonical glycosylation sequences N-X-S/T can be modified more efficiently with mutant NGTs identified using our high throughput experimentation.

### 5. Expanding X₊₂ Selectivity Beyond Canonical Glycosylation Motifs

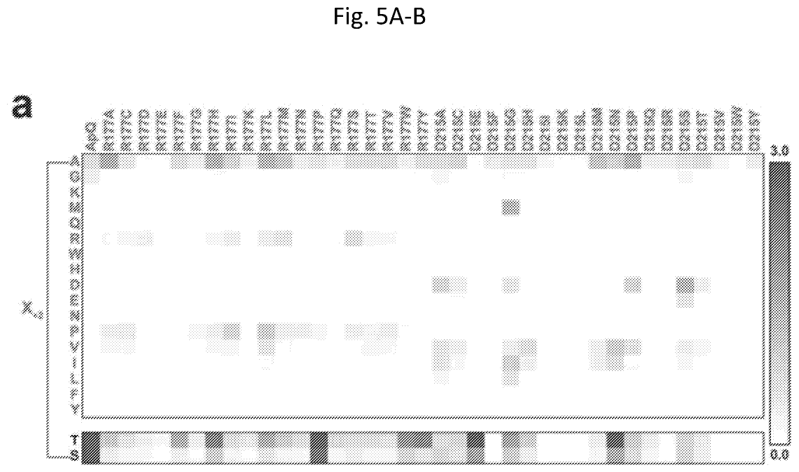

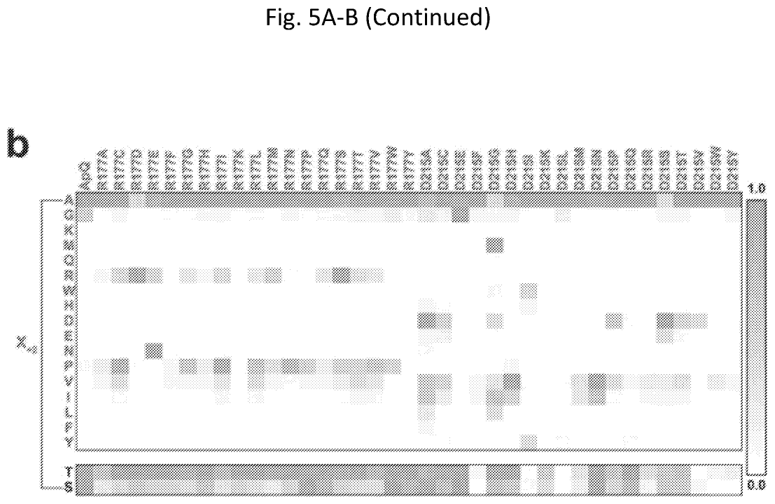

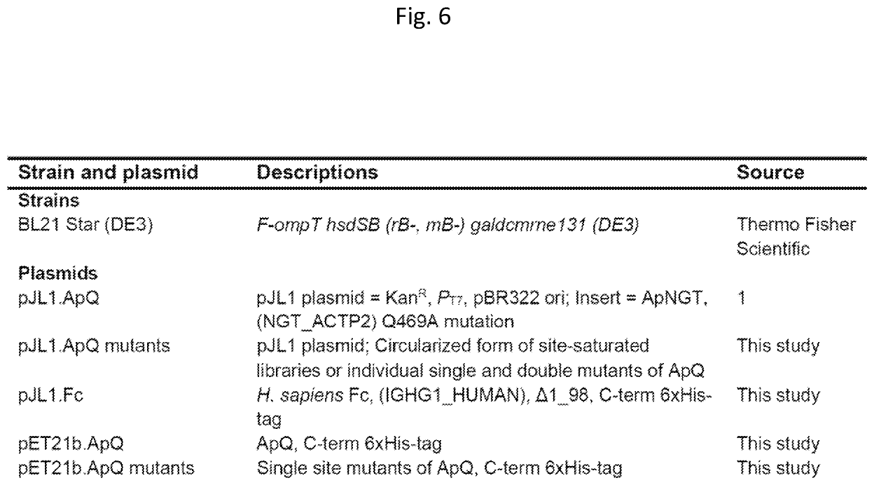

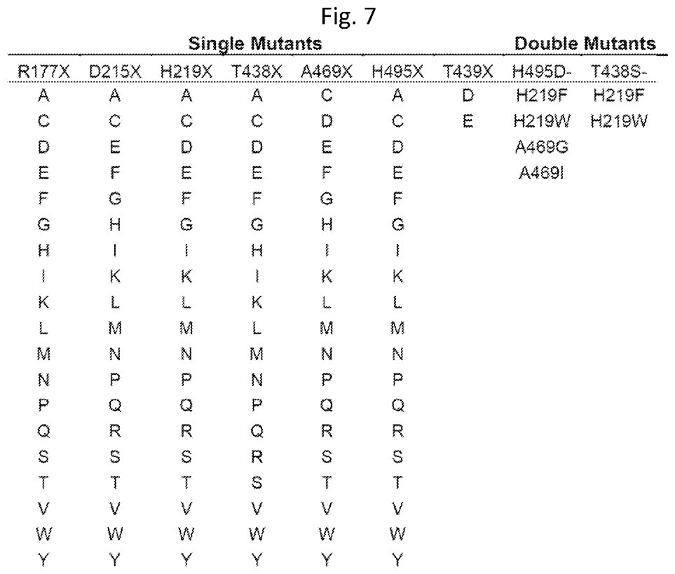

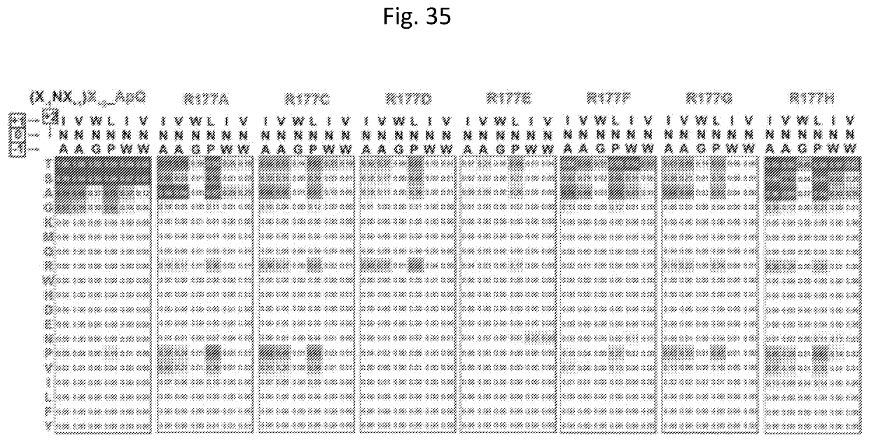

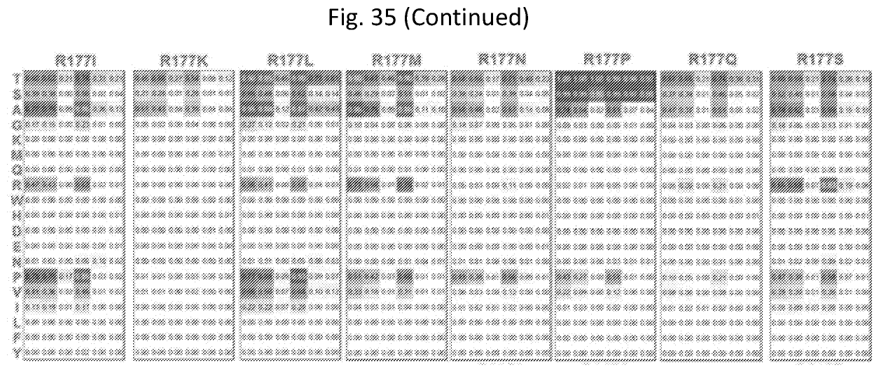

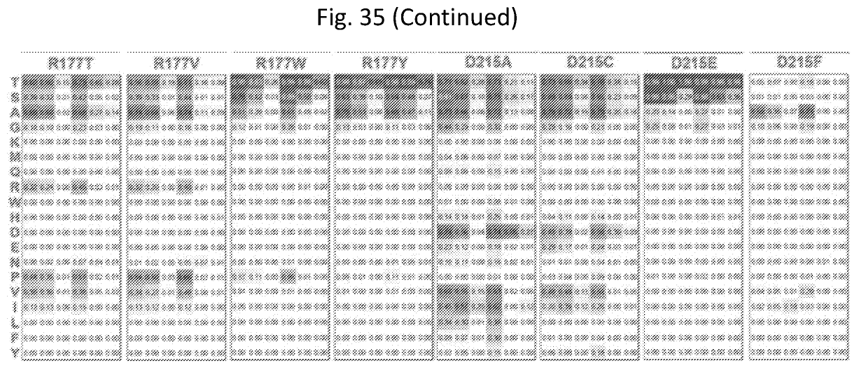

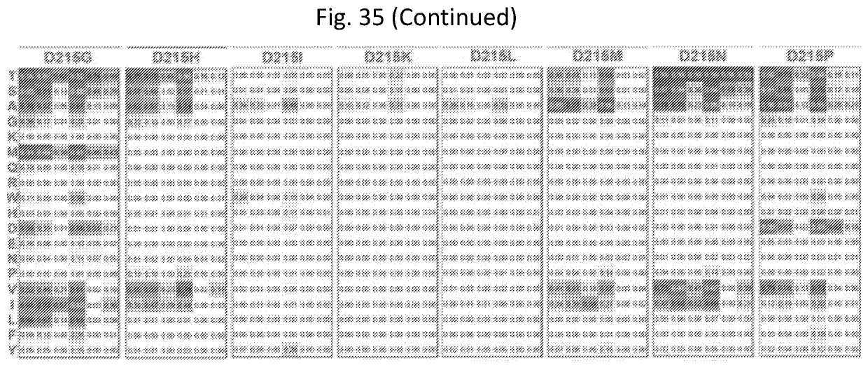

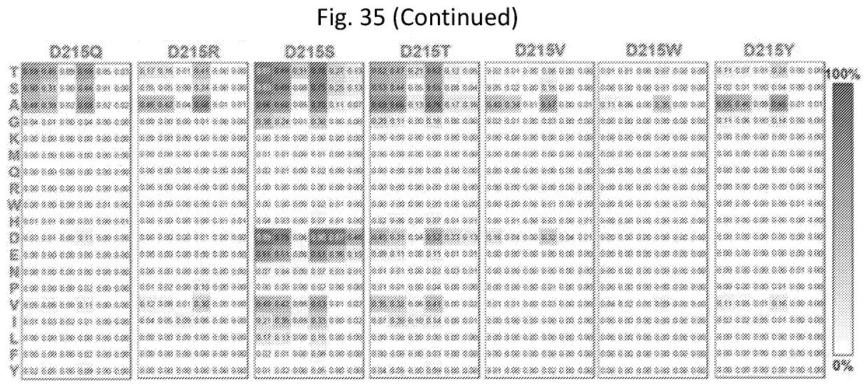

Thus far, the enzymatic N-glycosylation of a target protein has required a canonical N-X-S/T glycosylation motif that is either naturally occurring or introduced by altering primary amino acid sequences. This requirement presents a significant constraint to generalize enzymes for protein glycosylation. However, the introduction of new glycosylation sites without modifying primary amino acid sequences could also be enabled by engineered NGTs having a preference for sequences beyond the canonical glycosylation motif N-X-S/T. Therefore, the development of NGTs that do not require Ser or Thr at the X₊₂ position would significantly expand the range of sequences and proteins eligible for glycosylation. Based on their previous specificity screens and hypothesized peptide binding residues (FIGS. 18 and 21), the inventors sought to discover mutants that can glycosylate peptides without Ser or Thr at the X₊₂ position by screening all individual mutants of R177 and D215 across a the peptide library of the form (X₋₁NX₊₁)X₊₂RC (FIG. 35). As expected, the inventors found that most mutants tolerated S/T at the X₊₂ position. However, the inventors also found that R177 individual mutants tolerated A/R/P/V, D215 individual mutants tolerated A/D/E/V/I/L, and ApQ tolerated A/G at the X₊₂ position (FIG. 5a). D215G exhibited the broadest promiscuity for X₊₂ amino acids and could modify sequences with A/M/D/V/I/L at relatively high efficiency, as well as G/Q/W/E/N/F/Y at medium efficiency. Interestingly, the inventors found that D215F/I/L/V lost their preference for peptides with S/T at X₊₂ (FIG. 5b). This information can be used to guide the choice of an NGT mutant to target a sequence with a given X₊₂ amino acid (FIG. 5a) or to selectively target sequences with one X₊₂ amino acid over another (FIG. 5b).

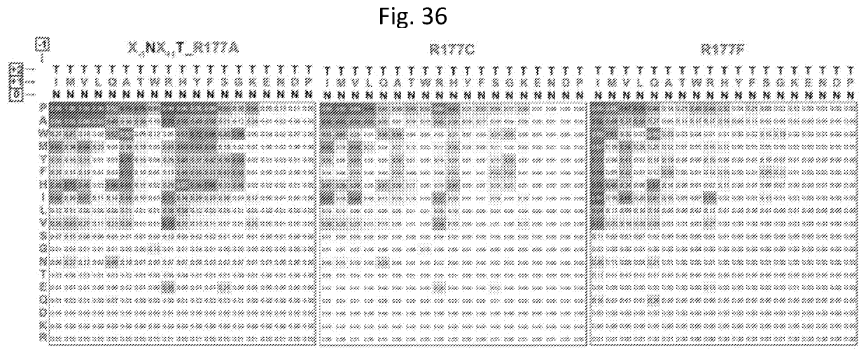

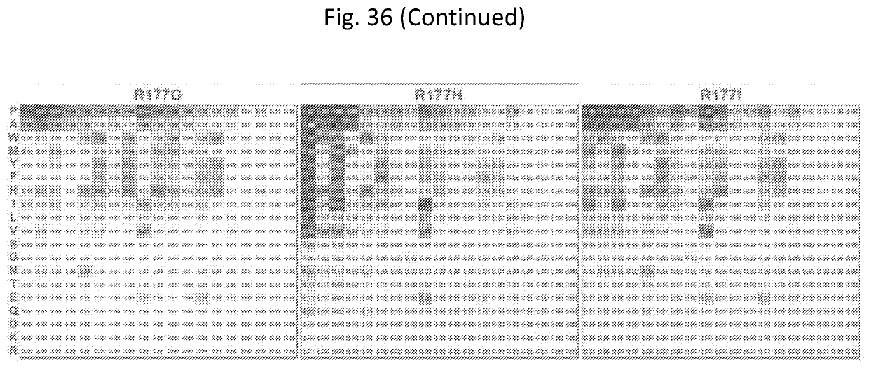

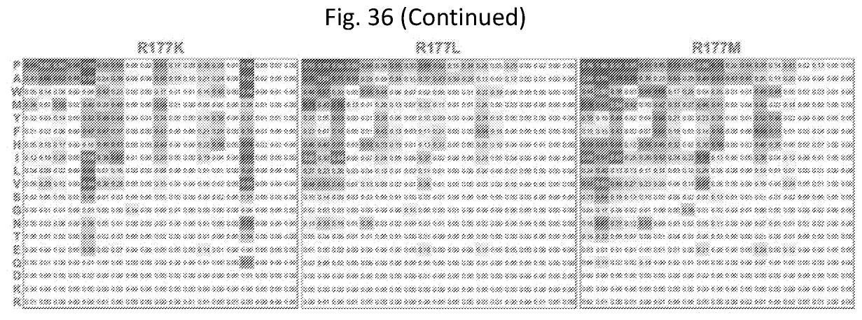

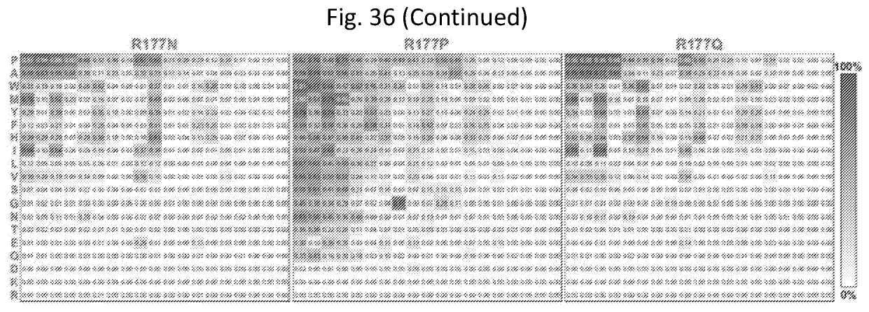

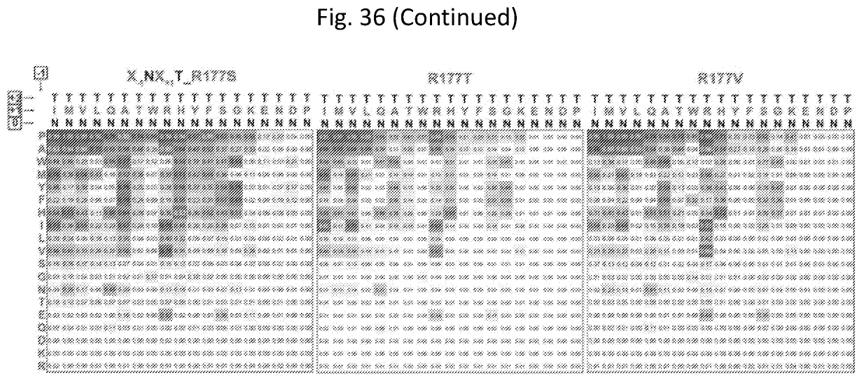

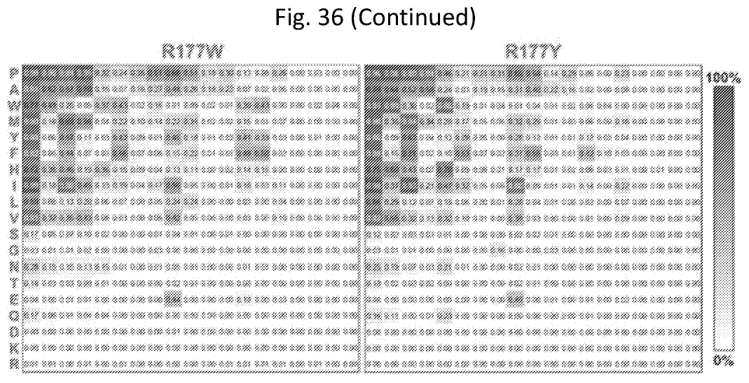

Finally, the inventors sought to explore which non-canonical sequences might be targeted with their newly discovered R177 and D215 mutants that exhibited expanded specificity at the X₊₂ position. In the previous SSVL screens, D215X exhibited little change in X₋₁ and X₊₁ specificity. Therefore, the X₋₁-N-X₊₁-TRC screen of D215X (FIG. 15) can be used to approximate the specificity of all D215 mutants for X₋₁ and X₊₁ combinations. However, the inventors also observed that R177X significantly altered the enzyme selectivity for the X₋₁ position. Therefore, the inventors screened all R177 mutants with X₋₁-N-X₊₁-TRC to determine which X₋₁ and X₊₁ combinations could be used with non-S/T amino acids at the X₊₂ position (FIG. 36). Overall, the inventors found that 10 non-S/T amino acids (A/G/M/R/D/E/P/V/I/L) at the X₊₂ position can be modified at relatively high efficiency on the peptide level. However, the inventors note that the modification of non-canonical sequences remains less efficient than that of the canonical sequences. Further engineering or evolution of NGTs targeting non-canonical peptide sequences will be required to achieve highly efficient modification of non-canonical sequences in therapeutic proteins.

### C. Discussion

In this work, the inventors present a systematic method to identify enzyme residues that determine specificity for each amino acid position of peptide substrates and the inventors use these sites as a starting point to develop a panel of specificity-distinct NGTs capable of modifying unique sets of substrate sequences. Their high throughput GlycoSCORES characterization technique enabled the screening of 123 individual NGTs through 52,894 independent reactions. To the inventors' knowledge, this is the most detailed glycosyltransferase engineering and characterization effort completed to date, surpassing the state-of-the-art¹⁹ by nearly fourfold. With minor adaptations to the workflow, this method of developing an enzymatic repertoire for modification of an entire substrate library should be applicable to other glycosyltransferases¹⁹, proteases²⁶, phosphatase³¹, deacetylases³², and other enzymes^{33,34}.

Two key features make this approach important. First, the rigorous characterization enabled the development of a panel of 14 NGTs that significantly expand the set of sequences available for glycosylation by bacterial enzymes. Of the 684 peptides within the canonical eukaryotic glycosylation motif (X₋₁-N-X₊₁-S/T where X₊₁≠P) that the inventors surveyed, 260 peptides were found to be modified with significantly higher efficiency by one of the 13 NGT mutants compared to ApQ (FIG. 10-11). These variants increase the percentage of sequences that can be glycosylated by NGTs with good efficiency (more than 80% modification with ˜0.5 μM NGT and 3-h reaction) from 45% to 66%. This expanded panel of NGTs permits the rational glycosylation of a sequence of interest by identifying the optimal NGT from the heatmap reported in FIG. 32. The inventors successfully applied this strategy to increase the modification of the therapeutic proteins IFNγ GM-CSF, and Fc using the A469I, T438S, and H495D mutants of ApQ, respectively. The inventors also developed NGTs that can glycosylate or even prefer sequences outside of the canonical N-X-S/T motif with non-S/T amino acids at the X₊₂ position. This discovery widens the scope of glycoengineering, allowing researchers to investigate how glycans can be used to improve the properties of a more diverse set of proteins without the need to modify their native amino acid sequences. Notably, many of the mutants discovered in this work possess quite distinct substrate specificities, which may enable the site-specific control of glycosylation structures at multiple sequences within a single protein by sequential modification²² to enable the precise engineering of synergistic glycan interactions.

Second, this work is important because it highlights the importance of high throughput experimentation. While typical directed evolution workflows lead to enzymes capable of performing a single reaction, this approach can be used to develop enzymes having multiple properties. Indeed, the parallel approach to activity monitoring enabled by GlycoSCORES (combination of CFPS and SAMDI-MS) results in variants having unique specificities—which enable a pattern of activity on many different substrates—and affords new possibilities for glycoengineering.

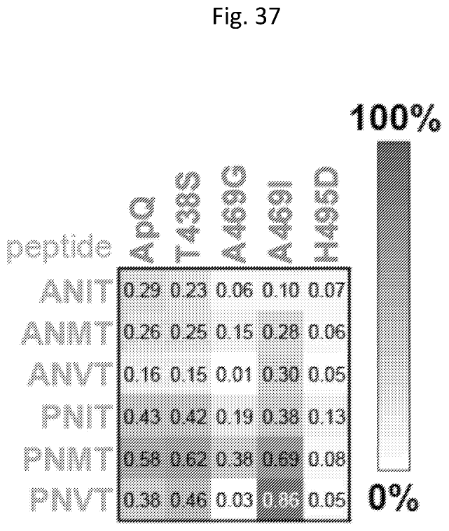

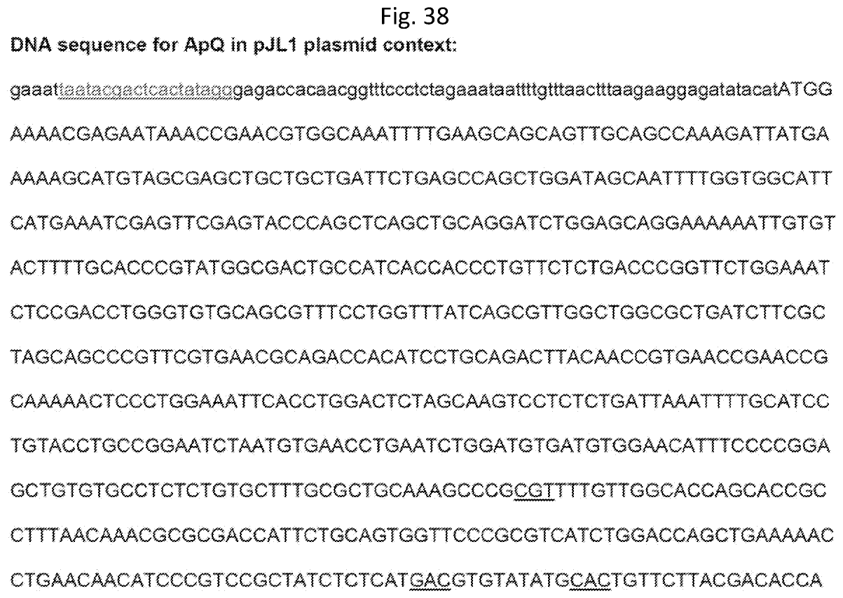

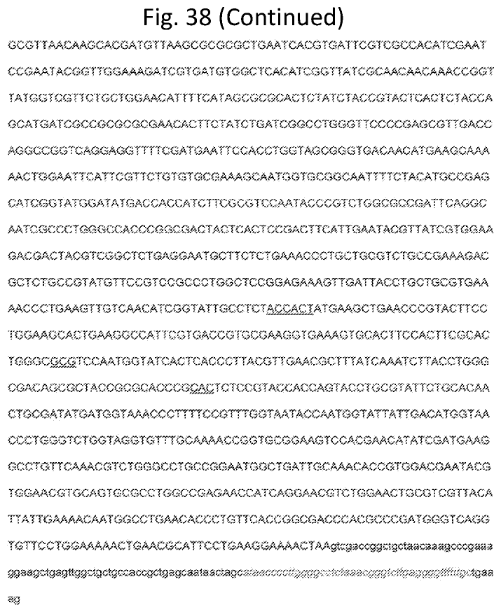

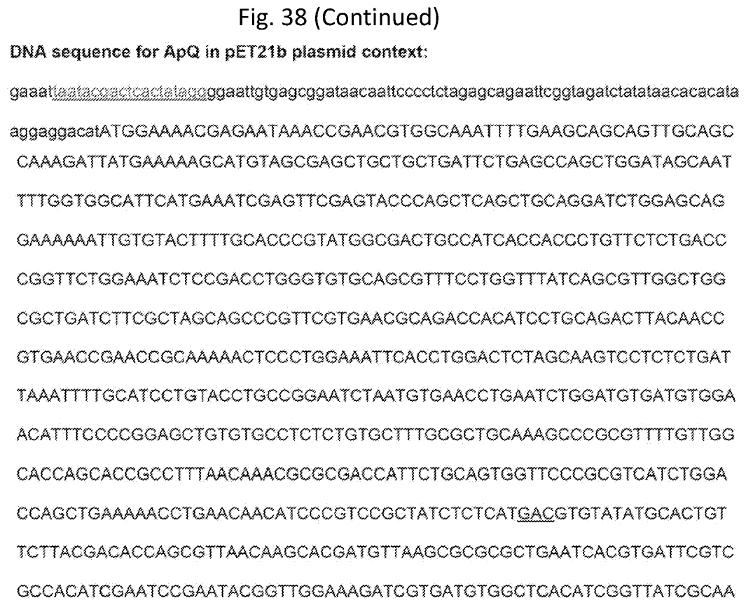

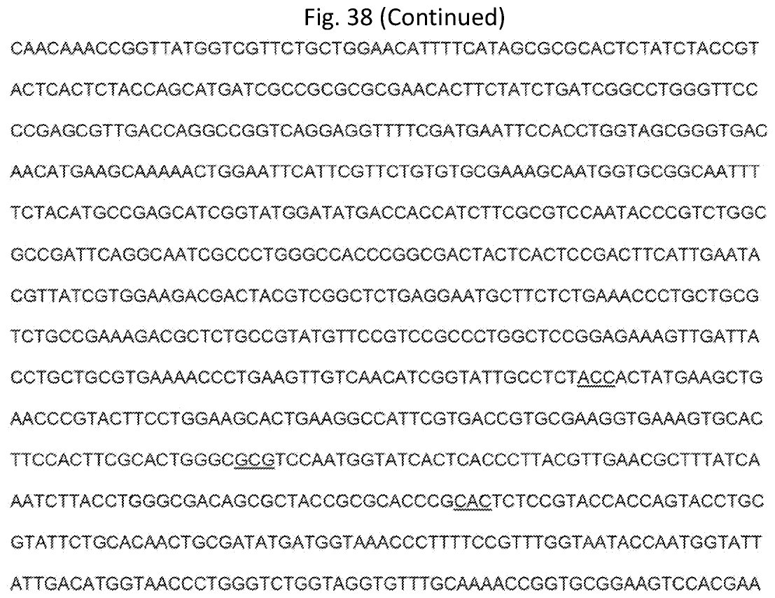

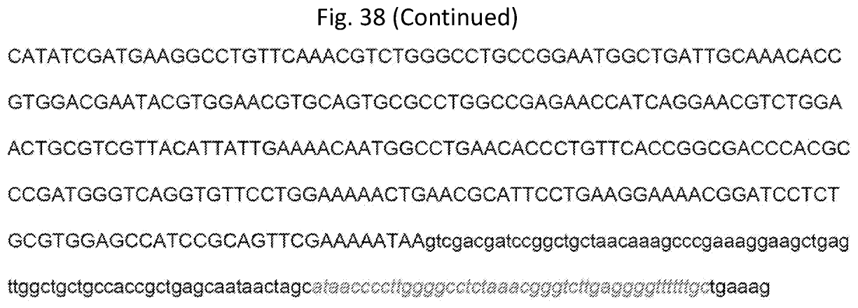

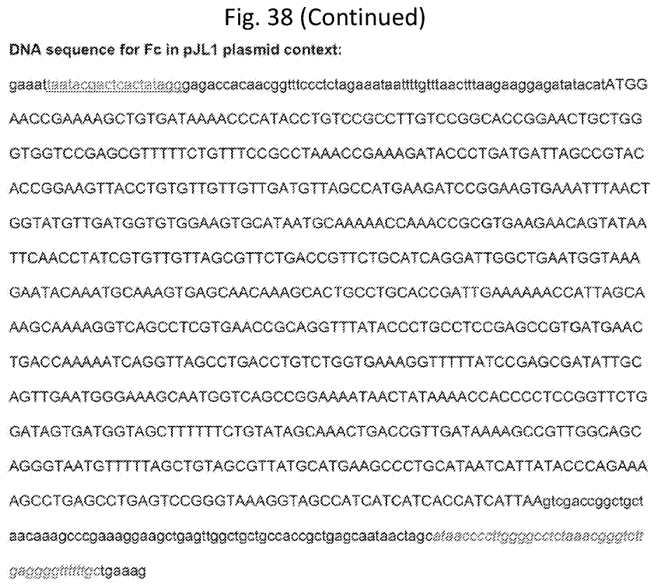

While this work focuses on the initiation step of glycosylation, many enzymatic and chemoenzymatic technologies have been developed to elaborate the monosaccharide installed by NGTs into human-like or otherwise useful glycans. For example, chemoenzymatic methods using endoglycosidases and chemically synthesized oxazoline donors can be used to install full-length human glycans^{22, 23}. These full-length human glycans may provide increased serum half-life for proteins³⁵ or provide ways to tune other therapeutic effects through the installation of a homogeneous N-glycan on the Fc region of human IgG^{4, 12}. Biosynthetic approaches to extend the glucose installed by NGTs to diverse and useful glycans, including polysialic acid, have also been developed²¹. Notably, the reducing end sugar of human N-glycans is N-acetylglucosamine (GlcNAc), rather than the glucose installed by NGTs. The effect of this difference on glycoprotein immunogenicity and other properties is unknown and will need to be assessed for each application. A two-step method using ApQ to install N-glucosamine (GlcN) and the acetyltransferase GlmA has already been developed³⁰, and the inventors are currently working on discovering NGT mutants that can more efficiently transfer GlcN and even GlcNAc. For example, the inventors found that A469I and T438S can modify some peptides with GlcN more efficiently than ApQ (FIG. 37). Several other highly active mutants discovered in this work can also modify peptides with GlcN (FIG. 37).

Despite significantly increasing the range of sequences that can be glycosylated, the inventors do recognize that the protein structure surrounding a targeted glycosylation sequence can affect the efficiency of modification. NGTs normally act post-translationally on folded proteins and therefore sites that are buried or rigidly locked into secondary or tertiary structure may not be available for modification by NGTs. Thus, some targets may require the use of other existing glycosylation methods using oligosaccharyltransferases (OSTs). While OSTs are complex integral membrane proteins and require lipid-linked oligosaccharide (LLO) substrates, they are capable of co-translational modification on unfolded sites^{36, 37}. Despite recent efforts to engineer or discover OSTs with expanded specificities^{38, 39}, it is usually still necessary to install a glycosylation tag (GlycTag) by extending or otherwise alter the primary amino acid sequence of the target protein in order to achieve glycosylation^{17, 40}. Therefore, a comprehensive engineering of OSTs similar to the work performed here for NGTs is also urgently needed to expand the set of sequences permissible to modification with OSTs.

In summary, the inventors demonstrate the application of a high throughput experimentation to engineer glycosyltransferases by using LET-CFPS and SAMDI-MS for parallelized generation and characterization of many enzyme mutants on a broad range of substrates. Using this method, the inventors have developed a panel of rigorously characterized, readily expressed, fully soluble N-glycosylation enzymes with unique activities that will serve as a valuable resource for the glycoengineering community. The inventors expect that this panel of NGTs will be especially useful in the bacterial or in vitro glycoengineering of protein therapeutics because it alleviates the need to alter primary amino acid sequences to achieve glycosylation for many protein therapeutics. Ultimately, the inventors' approach is poised to facilitate basic understanding in glycoscience and enable new applications in glycoengineering.

### E. Methods

### 1. Peptide Library Synthesis and SAMDI Screening

All peptide libraries were synthesized with N-acetyl and C-amide as previously described¹⁹. The average concentration of each peptide library was also determined as before¹⁹, as well as the calculation of average relative ionization factors (RIFs). SAMDI plates were also prepared and used for peptide screening as before¹⁹.

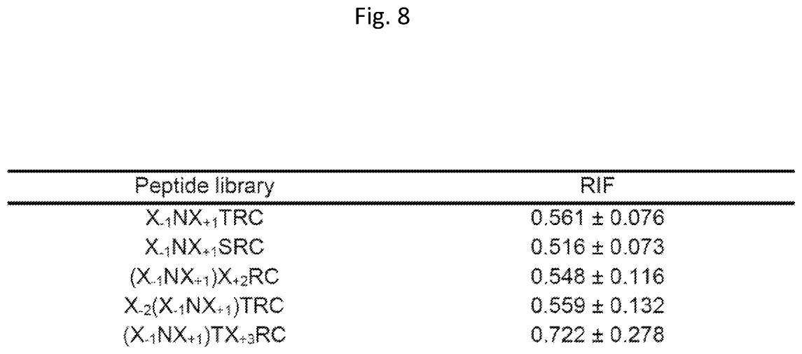

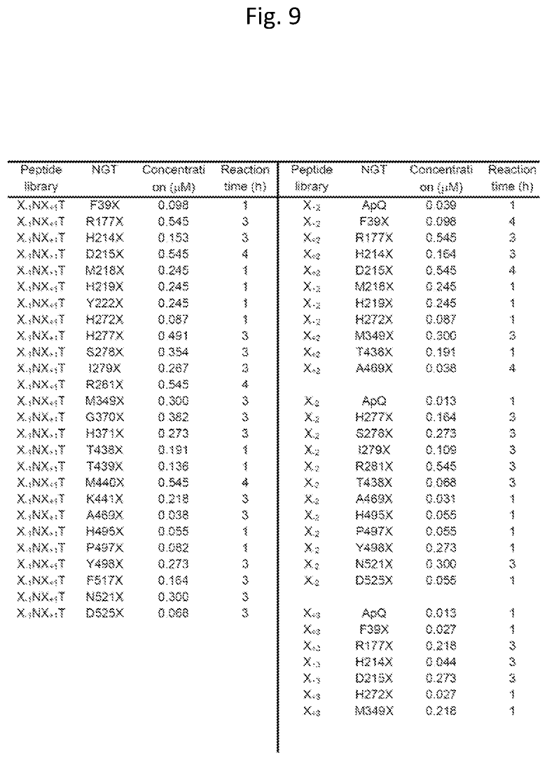

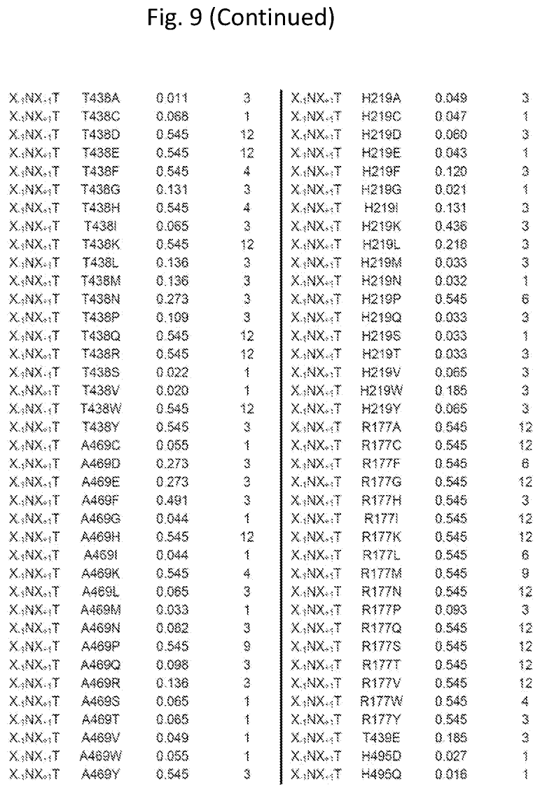

For peptide screening, 50 μM peptide was reacted with the indicated concentration of NGT, purified or produced in LET-CFPS, and 2.5 mM UDP-Glc in 100 mM HEPES (pH 8.0) and 500 mM NaCl at 30° C. for indicated time. Screenings for UDP-GlcN modification of peptides were completed similarly using 50 μM peptide with the 1.09 μM NGT produced in LET-CFPS and 2.5 mM UDP-GlcN (custom synthesized at Chemily Glycoscience) in 100 mM HEPES (pH 8.0) and 500 mM NaCl at 30° C. for 12 h. After the IVG reaction, TCEP-resin (Pierce) was added and incubated at 37° C. for 1 h. 2 μL solutions of these reduced IVGs were added to the islands of a 384-well maleimide-functionalized SAMDI plate and incubated at room temperature for 0.5 h. Because the reaction is not quenched during the 1-h TCEP reduction and 0.5-h SAMDI incubation steps, the inventors approximated this time as an additional 1 h of reaction for approximate k_{cat}/K_{M} calculations. The SAMDI plate was then washed with water, ethanol, water, and ethanol before being dried with nitrogen flow. 10 mg/mL 2′,4′,6′-trihydroxyacetophenone monohydrate (THAP; Sigma-Aldrich) in acetone was applied onto SAMDI plate as the matrix. The plate was then analyzed with a matrix-assisted laser desorption ionization time-of-flight (MALDI-TOF) mass spectrometry using an AB SCIEX 5800 TOF/TOF instrument. Spectra was processed with Applied Biosystems SciEx Time of Flight Series Explorer Software version 4.1.0. All peptide library screenings were completed with n=1. Modification efficiencies were determined using spectral peak ratios adjusted by relative ionization factor (RIF)¹⁹ (FIG. 8) except the data for UDP-GlcN, which show the relative intensity of glycopeptide (product peak) to all peptides (substrate and product peaks).

2. Synthesis of linear and plasmid SSVLs. Linear DNA for 26 SSVLs of ApQ were synthesized by Twist Bioscience within a linearized form of the pJL1 cell-free expression backbone opened within the kanamycin resistance gene such that recircularization with SapI restriction enzyme sites installed at the 5′ and 3′ ends would result in pJL1.ApQ. Plasmid forms of SSVLs were generated by circularizing these linear libraries using the Golden Gate assembly method with the SapI restriction enzyme⁴¹. 30 ng (6 μl) of each linear library was incubated with 1 μl each of 10,000 U/mL SapI restriction enzyme, 10 mM ATP, 10× CutSmart Buffer, and 2,000,000 T4 ligase (all products from New England Biolabs). Circularization reactions were carried out with 30 cycles of 1 min at 37° C. and 1 min at 16° C. followed by 5 min at 65° C. Completed circularization reactions were then transformed into DH5α electrocompetent cells and plated on LB (KAN+). All plates produced more than 100 colonies (ensuring 5-fold coverage of the library). After overnight growth, these LB (KAN+) plates were washed with 5 mL of LB media and miniprepped to generate plasmid libraries.

3. Isolating Individual NGT Mutants from SSVLs.

The plasmid SSVLs of selected residues, R177, D215, H219, T438, A469 and H495, were transformed into DH5α high efficiency chemically competent cells (New England Biolabs) by heat shock followed by incubation on LB agar plate (KAN+). More than 50 clones were picked from each SSVL transformation, cultured in LB (KAN+) media, miniprepped and sequenced to isolate all 19 individual mutants.

### 4. Constructions of Single and Double Mutants.

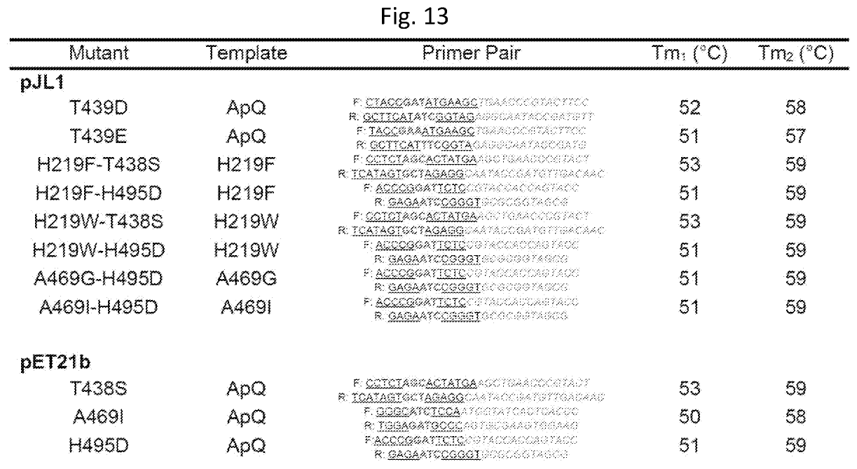

Single mutants of ApQ were generated using single-site PCR mutagenesis of a pJL1.ApQ template as previously reported⁴². Briefly, 25 μL PCR reactions were performed which contained 12.5 μL Q5 hot start high-fidelity 2× master mix (New England Biolabs), 1 ng template, 500 nM primer pair. The primers and Tm temperatures for these PCRs are listed in FIG. 13. The PCR was initiated at 98° C. for 30 s; followed by 15 circles of 98° C. for 10 s, Tm₂ for 30 s and 72° C. for 2 min; finished at Tm₁ for 1 min and 72° C. for 4 min. After the PCR, 2.5 μL 10× CutSmart buffer and 0.5 μL DpnI (New England Biolabs) was added and incubated at 37° C. for 2 h. Agarose gel electrophoresis was used to confirm the production of full-length PCR product (˜3.5 kb). The PCR solutions (after DpnI treatment) were transformed in DH5α high efficiency chemically competent cells by heat shock and applied on LB agar plate (KAN+). Two clones for each plate were picked, cultured, miniprepped and sequenced. Double mutants were generated similarly except single mutants were used as the initial plasmid template.

### 5. LET-CFPS.

CFPS reactions were performed using crude lysate derived from E. coli strain BL21 Star (DE3) as previously described^{22, 43} using linear DNA expression templates produced by PCR rather than plasmids²⁸. Crude lysates for CFPS were prepared by growth, harvest, and lysis of BL21 Star (DE3) E. coli cells as previously described⁴³ using a total energy input of 640 J for lysis of 1 mL cell suspensions. LET-CFPS reactions were performed at 50 μL in 2.0 mL centrifuge tubes containing 1.2 mM ATP (pH 7.2), 0.85 mM GTP, UTP, and CTP (pH 7.2); 34 μg/mL folinic acid; 171 μg/mL of E. coli tRNA mixture; 2 mM of 20 standard amino acids; 0.33 mM nicotinamide adenine dinucleotide (NAD); 0.27 mM coenzyme-A (CoA); 1.5 mM spermidine; 1 mM putrescine; 4 mM sodium oxalate; 130 mM potassium glutamate; 10 mM ammonium glutamate; 8 mM magnesium glutamate; 57 mM HEPES (pH 7.2); 33 mM phosphoenolpyruvate (PEP, pH 7); 20% v/v NGT linear template; and 27% v/v of BL21 crude extracts. NGT linear template was generated in a PCR reaction and used directly without purification. The 60 μL PCR reactions contained 30 μL Q5 hot start high-fidelity 2× master mix, 1.2 ng template (linear SSVLs synthesized by Twist or individual mutant plasmids), 500 nM primer pair (ccacctctgacttgagcgtc and gcagtttcatttgatgctcgatg). The PCR was initiated at 98° C. for 30 s; followed by 36 circles of 98° C. for 10 s, 65° C. for 30 s and 72° C. for 1 min; finished at 72° C. for 2 min. All reagents used in CFPS were purchased in Sigma-Aldrich except E. coli total tRNA mixture from strain MRE600 and PEP (Roche Applied Science). All CFPS reactions were carried out at 22° C. for 20 h. After the reaction, 1:1 v/v of 2× Roche complete protease inhibitor cocktail and 5 mM EDTA were added to the CFPS solutions, and the solutions were flash-frozen in liquid nitrogen and stocked at −80° C. for future use.

6. Approximate k_{cat}/K_{M} Calculation for Peptide Library X₋₁-N-X₊₁-TRC.

According to previous studies, the K_{M} of optimized peptides of the form X₋₂-X₋₁-N-X₊₁-T-X₊₃-RC for ApNGT are greater than 0.5 mM¹⁹, and longer peptide substrates generally exhibit lower K_{M} values than shorter peptides²⁰. Previous reports have also found that the K_{M} of ApQ differs by approximately 1.5-fold compared to ApNGT²⁰. Based on these findings, the inventors used a concentration of peptides (50 μM) that is much smaller than the K_{M} for the NGT variants used in this study. Thus, the inventors use equation k_{cat} K_{M}=−ln(1−Y)/c/t to approximate the value of k_{cat}/K_{M}, in which Y is the modification for peptides, c is the concentration of enzyme used, and t is the reaction time for glycosylation. Heat maps showing values of −ln(1−Y), which is in direct proportion to k_{cat}/K_{M} of the peptides, c(NGT), and t, were generated from modification heat maps.

While approximate, this calculation allowed us to compare the activity of all peptide-enzyme combinations with more than 10-fold fewer reactions than would have been required to rigorously determine k_{cat}/K_{M} values. The inventors also note that average k_{cat}/K_{M} values from a complete library of 361 conditions will be much more accurate than individual k_{cat}/K_{M} approximations. The inventors used the average k_{cat}/K_{M} to compare the activity between each mutant or SSVLs across different enzyme concentrations and reaction times. Because the apparent average k_{cat}/K_{M} was affected by the value of average −ln(1−Y) (FIG. 16A-D), the relative average k_{cat}/K_{M} of mutants were compared to an ApQ screen that yielded the same value of average −ln(1−Y). The optimal NGTs chosen for glycosylation of the whole set of canonical eukaryotic glycosylation sequences (X₋₁-N-X₊₁-T/S-RC) (FIGS. 27-28), were determined by calculating the approximate k_{cat}/K_{M} for each peptide-NGT combination and choosing the NGT mutant that provided the highest value. The selected NGTs (including ApQ) were screened with the same conditions. Specifically, 0.545 μM NGT was produced in LET-CFPS and combined with 2.5 mM UDP-Glc and 50 μM peptide before incubation at 30° C. for 3 h.

### 7. Analysis of the Mean Percentage Differences Between SSVLs or Single Mutants and ApQ.

To serve as references to compare the specificities of SSVLs or single mutants to the specificity of ApQ, several X₋₁-N-X₊₁-TRC heatmaps were analyzed after IVGs with various amounts of ApQ, yielding heatmaps with various averages of −ln(1−Y), where Y is modification. Using linear interpolation between two of these ApQ reference heatmaps, a theoretical ApQ heatmap with the same average −ln(1−Y) as the measured heatmap for a given SSVL or single mutant was generated (reference ApQ heatmaps and description of calculation process found in FIG. 16A-D). The inventors then calculated the percentage difference between the average of the −ln(1−Y) values for all 19 peptides with a given X₋₁ amino acid lane in the theoretical ApQ heatmap (defined as Ave(ApQ)) and the average of the −ln(1−Y) values for all 19 peptides with a given X₋₁ amino acid lane in the measured mutant heatmap (defined as Ave(X)) using the equation 2*|Ave(X)−Ave(ApQ)| (Ave(X)+Ave(ApQ)). And the average of percentage differences for all 19 X₋₁ amino acid rows gives the mean percentage difference of X₋₁. The X₊₁ mean percentage difference values were calculated similarly. The mean percentage difference for the whole X₋₁-N-X₊₁-TRC library is the average of the mean percentage differences for all X₋₁ and X₊₁ lanes. This calculation method was used to generate mean percentage differences shown in FIGS. 1A-D, -2A-C and FIG. 16A-D.

### 8. Combinatorial Comparison of SSVL and Single Mutant Specificities by Mean Percentage Differences.

To compare the specificities between individual mutants or SSVLs to each other, the inventors calculated the mean percentage difference between any two mutants from mean percentage differences of each one to ApQ. The numeric value, not the absolute value, of 2*(Ave(X)−Ave(ApQ))|/(Ave(X)+Ave(ApQ)) for each X₋₁ or X₊₁ lane was calculated as above for each mutant and defined as PD₁ for mutant 1 and PD₂ for mutant 2. The percentage difference between mutant 1 and 2 at each X₋₁ or X₊₁ lane was then calculated using the equation |PD₁−PD₂|/(1−PD₁*PD₂). Using this method, the inventors calculated the mean percentage differences between all SSVLs or isolated mutants at each residue for X₋₁, X₊₁ or the entire library, respectively. This calculation is based on the assumption that the percentage difference for each X₋₁ and X₊₁ lane between two NGTs remains unchanged when determined from heatmaps with different average values of −ln(1−Y). This calculation method was used to generate mean percentage differences in FIGS. 17 and 26.

9. ApQ and Mutant Plasmid Construction, Expression in E. coli and Purification.

ApQ mutant constructs were generated in the pET21b vector for in vivo expression and purification. Mutagenesis was performed the same way as described above for in vitro constructs in the pJL1 vector. Primers and Tm used are listed in FIG. 13. NGTs were purified as described previously with minor modifications¹⁹. Briefly, BL21 Star (DE3) chemically competent cells were transformed with pET21b.ApQ or mutant plasmids by heat shock. An overnight culture was inoculated in LB (CARB+) media. Fresh LB (CARB+) was inoculated at initial OD600=0.08, and the cells were grown at 37° C. at 250 rpm to 0.6-0.8 OD and induced with 1 mM isopropyl β-d-1-thiogalactopyranoside (IPTG) for 6 h at 30° C. The cells were pelleted by centrifugation at 5,000×g for 10 min at 4° C., resuspended in Buffer 3 (20 mM Tris-HCl and 250 mM NaCl, pH 8.0), pelleted again by centrifugation at 8,000×g for 10 min at 4° C., and flash frozen at −80° C. The pellets were then thawed and resuspended in 5 mL Buffer 3 per gram wet pellet weight and supplemented with 1 mg/mL lysozyme (Sigma), 1 μL benzonase (Millipore), and 1× Halt protease inhibitor (Thermo Fisher Scientific). Cells were then lysed by single pass homogenization at 21,000 psig (Avestin) and centrifuged at 13,000×g for 20 min at 4° C. Imidazole was added to the supernatant to a final concentration of 20 mM. The supernatant was applied to 1 mL Ni-NTA agarose resin (Qiagen) equilibrated with Buffer 3 with 20 mM imidazole. Following a 1-h incubation, the resin was washed once with 5 column volumes of Buffer 3 with 20 mM imidazole, washed twice with 5 column volumes of Buffer 3 with 30 mM imidazole and once with 5 column volumes of Buffer 3 with 40 mM imidazole. The protein was eluted in 1 column volume of Buffer 3 with 500 mM Imidazole. The elution was dialyzed against 50 mM HEPES, 200 mM NaCl, pH 7.0 and flash frozen at −80° C. Protein concentration was quantified with a NanoDrop UV-Vis spectrophotometer (Thermo Fisher) using the following parameters, Molecular weight: 71502.50 Da, Extinction coefficient: 63260 M⁻¹cm⁻¹.

### 10. In Vitro Protein Glycosylation and LC-qTOF Analysis of Tryptic Glycopeptides.

10 μM IFNγ (Millipore) or GM-CSF (R&D Systems) was reacted with 5 μM purified ApQ or mutants and 5 mM UDP-Glc in Buffer 1, 50 mM NaH₂PO₄ (pH 8) and 300 mM NaCl. The reactions were carried out at 30° C. for 12 h. After the reaction, 10 μL solutions were diluted to 30 μL and dialyzed with Pierce 96-well microdialysis plate (3.5 k MWCO) against 1:4 diluted Buffer 1 for 8 h at room temperature. Dialyzed solutions were added with 1 μL 0.5 mg/mL Trypsin (Pierce) in 1 mM HCl and incubated at 37° C. for 16 h. 1 μL of 0.25 mM DTT was added to the reaction before resting it on ice for 1 h. The tryptic glycopeptides were analyzed as previously described²². Briefly, 5-10 μL of trypsinized samples were injected into a Bruker Elute UPLC system equipped with an ACQUITY UPLC Peptide BEH C18 Column, 300 Å, 1.7 μm, 2.1 mm×100 mm (186003686 from Waters Corporation) with a 10 mm guard column (186004629 Waters Corporation) coupled to an Impact-II UHR TOF Mass Spectrometer (Bruker Daltonics, Inc.). The chromatographic separation method used 100% water with 0.1% formic acid as solvent A and 100% acetonitrile with 0.1% formic acid as solvent B. Chromatography was completed using 100% A for 1 min and a gradient of 0% to 50% B for 4 min. The flow rate was kept at 0.5 mL/min. Mass spectra in a range of 100-3000 Da were collected in 8 Hz. External calibration was performed for all spectra. The inventors used MS/MS to monitor the target peptides and glycopeptides with collision energy of 50 eV (spectra shown in FIG. 33A-C). Bruker Compass Data Analysis software version 4.1 was used to analyze the data. The targeted peak in extracted ion chromatograms of targeted peptide and glycopeptide masses were integrated to calculate the modification using % Area, Area(P)/(Area(S)+Area(P)). Results were listed at FIG. 12.

### 11. LET-CFPS Protein Expression and Glycosylation.

pJL1.Fc was expressed in LET-CFPS the same way as NGTs with the addition of 2 or 5 μM purified ApQ or H495D mutant and 5 mM UDP-Glc. After 6-h CFPS incubation at 30° C., 70 μL Buffer 1 with 5 mM imidazole was added into 50 μL CFPS solutions. The reactions were centrifuged with 12,000×g for 15 mins at 4° C., and supernatants were mixed with 30 μL His Dynabeads (Invitrogen) for a 10-min incubation. The beads were washed thrice with 120 μL Buffer 1 with 5 mM imidazole and eluted with 80 μL Buffer 1 with 500 mM imidazole. Elution solutions were dialyzed with Pierce 96-well microdialysis plate (3.5 k MWCO) against 1:4 diluted Buffer 1 for 8 h at room temperature. 1 μL 0.5 mg/mL Trypsin (Pierce) in 1 mM HCl was added for 40 μL dialyzed solutions and incubated at 37° C. for 16 h. 1 μL of 0.25 mM DTT was added to the reaction before resting it on ice for 1 h. LC-qTOF analysis was performed as described above.

When noted that the glycosylation of Fc was completed after CFPS (post-folding), Fc was expressed in LET-CFPS for 30° C. and 20-h incubation, then centrifuged and supplemented with 2 μM purified ApQ and 5 mM UDP-Glc. This IVG reaction was then incubated at 30° C. for 6 h. The purification, dialysis, trypsinization and LC-qTOF analysis of these reactions were performed as above.

12. t-Test and Data Analysis.

Two-tailed Student's t-tests and resulting p values were calculated in Microsoft Excel 2016. For all peptide library screens, only n=1 experiment was used. For peptide IVGs of target protein sequences, n=3 independent reactions were performed. For therapeutic protein modifications, n=2 or n=3 independent reactions were carried out as noted in dot-plots. In the data analysis of n>1, the average is presented and standard deviations (s.d.) are shown as error bars. All heatmaps were generated in Microsoft Excel 2016.

### E. References

1. Khoury, G. A., Baliban, R. C. & Floudas, C. A. Proteome-wide post-translational modification statistics: Frequency analysis and curation of the swiss-prot database. Sci. Rep. 1, 90 (2011).
2. Schwarz, F. & Aebi, M. Mechanisms and principles of n-linked protein glycosylation. Curr. Opin. Struc. Biol. 21, 576-582 (2011).
3. Sethuraman, N. & Stadheim, T. A. Challenges in therapeutic glycoprotein production. Curr. Opin. Biotech. 17, 341-346 (2006).
4. Li, T. et al. Modulating igg effector function by fc glycan engineering. Proc. Natl. Acad. Sci. U.S.A. 114, 3485-3490 (2017).
5. Murakami, M. et al. Chemical synthesis of erythropoietin glycoforms for insights into the relationship between glycosylation pattern and bioactivity. Sci. Adv. 2, e1500678 (2016).
6. Mimura, Y. et al. The influence of glycosylation on the thermal stability and effector function expression of human iggl-fc: Properties of a series of truncated glycoforms. Mol. Immunol. 37, 697-706 (2000).
7. Wissing, S. et al. Expression of glycoproteins with excellent glycosylation profile and serum half-life in cap-go cells. BMC Proc. 9, P12 (2015).
8. Elliott, S. et al. Enhancement of therapeutic protein in vivo activities through glycoengineering. Nat. Biotechnol. 21, 414-421 (2003).
9. Perlman, S. et al. Glycosylation of an n-terminal extension prolongs the half-life and increases the in vivo activity of follicle stimulating hormone. J. Clin. Endocrinol. Metab. 88, 3227-3235 (2003).
10. Valderrama-Rincon, J. D. et al. An engineered eukaryotic protein glycosylation pathway in Escherichia coli. Nat. Chem. Biol. 8, 434-436 (2012).
11. Keys, T. G. & Aebi, M. Engineering protein glycosylation in prokaryotes. Curr. Opin. Syst. Biol. 5, 23-31 (2017).
12. Lin, C.-W. et al. A common glycan structure on immunoglobulin g for enhancement of effector functions. Proc. Natl. Acad. Sci. U.S.A. 112, 10611-10616 (2015).
13. Wang, L.-X. & Amin, M. N. Chemical and chemoenzymatic synthesis of glycoproteins for deciphering functions. Chem. Biol. 21, 51-66 (2014).
14. Jaroentomeechai, T. et al. Single-pot glycoprotein biosynthesis using a cell-free transcription-translation system enriched with glycosylation machinery. Nat. Commun. 9, 2686 (2018).
15. Schwarz, F. et al. A combined method for producing homogeneous glycoproteins with eukaryotic n-glycosylation. Nat. Chem. Biol. 6, 264-266 (2010).
16. Guarino, C. & DeLisa, M. P. A prokaryote-based cell-free translation system that efficiently synthesizes glycoproteins. Glycobiology 22, 596-601 (2012).
17. Schoborg, J. A. et al. A cell-free platform for rapid synthesis and testing of active oligosaccharyltransferases. Biotechnol. Bioeng., 739-750 (2018).
18. Wacker, M. et al. N-linked glycosylation in Campylobacter jejuni and its functional transfer into e. Coli. Science 298, 1790-1793 (2002).
19. Kightlinger, W. et al. Design of glycosylation sites by rapid synthesis and analysis of glycosyltransferases. Nat. Chem. Biol. 14, 627-635 (2018).
20. Song, Q. et al. Production of homogeneous glycoprotein with multisite modifications by an engineered n-glycosyltransferase mutant. J. Biol. Chem. 292, 8856-8863 (2017).
21. Keys, T. G. et al. A biosynthetic route for polysialylating proteins in Escherichia coli. Metab. Eng. 44, 293-301 (2017).
22. Lin, L., Kightlinger, W., Hockenberry, A. J., Jewett, M. C. & Mrksich, M. Sequential glycosylation of proteins with substrate-specific n-glycosyltransferases. ACS Cent. Sci. In revision (2019).
23. Lomino, J. V. et al. A two-step enzymatic glycosylation of polypeptides with complex n-glycans. Bioorg. Med. Chem. 21, 2262-2270 (2013).
24. Weeks, A. M. & Wells, J. A. Engineering peptide ligase specificity by proteomic identification of ligation sites. Nat. Chem. Biol. 14, 50-57 (2018).
25. Schilling, O. & Overall, C. M. Proteome-derived, database-searchable peptide libraries for identifying protease cleavage sites. Nat. Biotechnol. 26, 685-694 (2008).
26. Wood, S. E. et al. A bottom-up proteomic approach to identify substrate specificity of outer-membrane protease ompt. Angew. Chem. Int. Ed. 129, 16758-16762 (2017).
27. Gurard-Levin, Z. A., Kim, J. & Mrksich, M. Combining mass spectrometry and peptide arrays to profile the specificities of histone deacetylases. ChemBioChem 10, 2159-2161 (2009).
28. Schinn, S. M., Broadbent, A., Bradley, W. T. & Bundy, B. C. Protein synthesis directly from pcr: Progress and applications of cell-free protein synthesis with linear DNA. New Biotechnol. 33, 480-487 (2016).
29. Kawai, F. et al. Structural insights into the glycosyltransferase activity of the Actinobacillus pleuropneumoniae hmwlc-like protein. J. Biol. Chem. 286, 38546-38557 (2011).
30. Xu, Y. et al. A novel enzymatic method for synthesis of glycopeptides carrying natural eukaryotic n-glycans. Chem. Commun. 53, 9075-9077 (2017).
31. Szymczak, L. C., Huang, C. F., Berns, E. J. & Mrksich, M. Combining samdi mass spectrometry and peptide arrays to profile phosphatase activities. Methods Enzymol. 607, 389-403 (2018).
32. Kuo, H. Y., DeLuca, T. A., Miller, W. M. & Mrksich, M. Profiling deacetylase activities in cell lysates with peptide arrays and samdi mass spectrometry. Anal. Chem. 85, 10635-10642 (2013).
33. Kornacki, J. R., Stuparu, A. D. & Mrksich, M. Acetyltransferase p300/cbp associated factor (pcaf) regulates crosstalk-dependent acetylation of histone h3 by distal site recognition. ACS Chem. Biol. 10, 157-164 (2015).
34. Houseman, B. T., Huh, J. H., Kron, S. J. & Mrksich, M. Peptide chips for the quantitative evaluation of protein kinase activity. Nat. Biotechnol. 20, 270-274 (2002).
35. Kontermann, R. E. Strategies for extended serum half-life of protein therapeutics. Curr. Opin. Biotech. 22, 868-876 (2011).
36. Wild, R. et al. Structure of the yeast oligosaccharyltransferase complex gives insight into eukaryotic n-glycosylation. Science, 545-550 (2018).
37. Lizak, C., Gerber, S., Numao, S., Aebi, M. & Locher, K. P. X-ray structure of a bacterial oligosaccharyltransferase. Nature 474, 350-355 (2011).
38. Ollis, A. A. et al. Substitute sweeteners: Diverse bacterial oligosaccharyltransferases with unique n-glycosylation site preferences. Sci. Rep. 5, 15237 (2015).
39. Ollis, A. A., Zhang, S., Fisher, A. C. & DeLisa, M. P. Engineered oligosaccharyltransferases with greatly relaxed acceptor-site specificity. Nat. Chem. Biol. 10, 816-822 (2014).
40. Fisher, A. C. et al. Production of secretory and extracellular n-linked glycoproteins in Escherichia coli. Appl. Environ. Microbiol. 77, 871-881 (2011).
41. Engler, C., Kandzia, R. & Marillonnet, S. A one pot, one step, precision cloning method with high throughput capability. PLoS ONE 3, e3647 (2008).
42. Liu, H. & Naismith, J. H. An efficient one-step site-directed deletion, insertion, single and multiple-site plasmid mutagenesis protocol. BMC Biotechnol. 8, 91 (2008).
43. Kwon, Y.-C. & Jewett, M. C. High-throughput preparation methods of crude extract for robust cell-free protein synthesis. Sci. Rep. 5, 8663 (2015).

### ADDITIONAL REFERENCES

Lin, L., Kightlinger, W., Hockenberry, A. J., Jewett, M. C. & Mrksich, M. Sequential glycosylation of proteins with substrate-specific n-glycosyltransferases. ACS Cent. Sci. In revision (2019).
Kawai, F. et al. Structural insights into the glycosyltransferase activity of the Actinobacillus pleuropneumoniae hmwlc-like protein. J. Biol. Chem. 286, 38546-38557 (2011).
Naegeli, A. et al. Substrate Specificity of Cytoplasmic N-Glycosyltransferase. Journal of Biological Chemistry 289, 24521-24532 (2014).
Naegeli, A. et al. Molecular analysis of an alternative N-glycosylation machinery by functional transfer from Actinobacillus pleuropneumoniae to Escherichia coli. The Journal of biological chemistry 289, 2170-2179 (2014).
Cuccui, J. et al. The N-linking glycosylation system from Actinobacillus pleuropneumoniae is required for adhesion and has potential use in glycoengineering. Open biology 7 (2017).
Song, Q. et al. Production of homogeneous glycoprotein with multi-site modifications by an engineered N-glycosyltransferase mutant. Journal of Biological Chemistry (2017).
Schwarz, F., Fan, Y.-Y., Schubert, M. & Aebi, M. Cytoplasmic N-Glycosyltransferase of Actinobacillus pleuropneumoniae Is an Inverting Enzyme and Recognizes the NX(S/T) Consensus Sequence. Journal of Biological Chemistry 286, 35267-35274 (2011).
Kightlinger, W. et al. Design of glycosylation sites by rapid synthesis and analysis of glycosyltransferases. Nat. Chem. Biol. 14, 627-635 (2018).
Keys, T. G. et al. A biosynthetic route for polysialylating proteins in Escherichia coli. Metab. Eng. 44, 293-301 (2017).
Tytgat, H. L. P., Lin, C., Levasseur, M. D. et al. Cytoplasmic glycoengineering enables biosynthesis of nanoscale glycoprotein assemblies. Nat Commun 10, 5403 (2019).
Ban, L. et al. Discovery of glycosyltransferases using carbohydrate arrays and mass spectrometry. Nature chemical biology 8, 769-773 (2012).
Laurent, N., et al. (2008). “Enzymatic Glycosylation of Peptide Arrays on Gold Surfaces.” Chembiochem 9(6): 883-887.
Kightlinger, W. et al. Design of glycosylation sites by rapid synthesis and analysis of glycosyltransferases. Nat. Chem. Biol. 14, 627-635 (2018).
Guarino, C., & DeLisa, M. P. (2012). A prokaryote-based cell-free translation system that efficiently synthesizes glycoproteins. Glycobiology, 22(5), 596-601.
Schoborg, J. A. et al. A cell-free platform for rapid synthesis and testing of active oligosaccharyltransferases. Biotechnol. Bioeng., 739-750 (2018).
Jaroentomeechai, T. et al. Single-pot glycoprotein biosynthesis using a cell-free transcription-translation system enriched with glycosylation machinery. Nat. Commun. 9, 2686 (2018).
Ollis, Anne A et al. “Engineered oligosaccharyltransferases with greatly relaxed acceptor-site specificity.” Nature chemical biology vol. 10,10 (2014): 816-22. doi:10.1038/nchembio.1609.
Song, Q. et al. Production of homogeneous glycoprotein with multi-site modifications by an engineered N-glycosyltransferase mutant. Journal of Biological Chemistry (2017).
Ollis, A. A.; Chai, Y.; Natarajan, A.; Perregaux, E.; Jaroentomeechai, T.; Guarino, C.; Smith, J.; Zhang, S.; DeLisa, M. P., Substitute sweeteners: diverse bacterial oligosaccharyltransferases with unique N-glycosylation site preferences. Scientific reports 2015, 5, 15237.
van Kasteren, S. I.; Kramer, H. B.; Gamblin, D. P.; Davis, B. G., Site-selective glycosylation of proteins: Creating synthetic glycoproteins. Nat. Protoc. 2007, 2 (12), 3185-3194.
van Kasteren, S. I.; Kramer, H. B.; Jensen, H. H.; Campbell, S. J.; Kirkpatrick, J.; Oldham, N. J.; Anthony, D. C.; Davis, B. G., Expanding the diversity of chemical protein modification allows post-translational mimicry. Nature 2007, 446 (7139), 1105-1109.
Wright, T. H.; Bower, B. J.; Chalker, J. M.; Bernardes, G. J.; Wiewiora, R.; Ng, W. L.; Raj, R.; Faulkner, S.; Vallee, M. R.; Phanumartwiwath, A.; Coleman, O. D.; Thezenas, M. L.; Khan, M.; Galan, S. R.; Lercher, L.; Schombs, M. W.; Gerstberger, S.; Palm-Espling, M. E.; Baldwin, A. J.; Kessler, B. M.; Claridge, T. D.; Mohammed, S.; Davis, B. G., Posttranslational mutagenesis: A chemical strategy for exploring protein side-chain diversity. Science 2016, 354 (6312), aag1465.
Yang, Q.; An, Y.; Zhu, S.; Zhang, R.; Loke, C. M.; Cipollo, J. F.; Wang, L.-X., Glycan Remodeling of Human Erythropoietin (EPO) Through Combined Mammalian Cell Engineering and Chemoenzymatic Transglycosylation. ACS Chemical Biology 2017, 12 (6), 1665-1673.
Giddens, J. P.; Lomino, J. V.; DiLillo, D. J.; Ravetch, J. V.; Wang, L.-X., Site-selective chemoenzymatic glycoengineering of Fab and Fc glycans of a therapeutic antibody. Proceedings of the National Academy of Sciences 2018, 115 (47), 12023-12027.

Patent references: US20140194345A1, US20180354997A1, CN 201610012793, U.S. Pat. Nos. 8,703,471, 8,999,668, US20050170452, US20060211085, US20060234345, US20060252672, US20060257399, US20060286637, US20070026485, US20070178551, WO2003056914A1, WO2004035605A2, WO2006102652A2, WO2006119987A2, WO2007120932A2.

In the foregoing description, it will be readily apparent to one skilled in the art that varying substitutions and modifications may be made to the invention disclosed herein without departing from the scope and spirit of the invention. The invention illustratively described herein suitably may be practiced in the absence of any element or elements, limitation or limitations which is not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention. Thus, it should be understood that although the present invention has been illustrated by specific embodiments and optional features, modification and/or variation of the concepts herein disclosed may be resorted to by those skilled in the art, and that such modifications and variations are considered to be within the scope of this invention.

All methods described herein can be performed in any suitable order unless otherwise indicated herein or otherwise clearly contradicted by context. The use of any and all examples provided herein, is intended merely to better illuminate the invention and does not pose a limitation on the scope of the invention unless otherwise claimed. No language in the specification should be construed as indicating any non-claimed element as essential to the practice of the invention.

Citations to a number of patent and non-patent references are made herein. The cited references are incorporated by reference herein in their entireties. In the event that there is an inconsistency between a definition of a term in the specification as compared to a definition of the term in a cited reference, the term should be interpreted based on the definition in the specification.

We claim:

1. A modified N-glycosyltransferase (NGT) selected from the following group or a homolog thereof:
(i) an Actinobacillus spp. (optionally a modified NGT of Actinobacillus pleuropneumoniae of SEQ ID NO:1) comprising one or more substitutions at amino acid positions F39, R177, H214, D215, M218, H219, Y222, H272, H277, S278, 1279, R281, M349, G370, H371, T438, T439, M440, K441, A469 (Q469), H495, P497, Y498, F517, N521, and D525;
(ii) a Kingella spp. (optionally a modified NGT of Kingella kingae of SEQ ID NO:2) comprising one or more substitutions at amino acid positions F42, R181, H218, D219, M222, H219, Y223, H276, H281, S282, 1283, R285, M354, G375, H376, T443, T444, M445, K446, A474 (Q474), H500, P502, Y503, F522, N526, and D530;
(iii) a Haemophilus spp. (optionally a modified NGT of Haemophilus pneumoniae of SEQ ID NO:3) comprising one or more substitutions at amino acid positions F68, R204, H241, D242, M245, H246, Y249, H298, H303, S304, 1305, R307, M375, G396, H397, T464, T465, M466, K467, A495 (Q495), H521, P523, Y524, F543, N547, and D551;
(iv) an Aggregatibacter spp. (optionally a modified NGT of Aggregatibacter aphrophilus of SEQ ID NO:4) comprising one or more substitutions at amino acid positions F39, R177, H214, D215, M218, H219, Y222, H270, H275, S276, 1277, R279, M348, G369, H370, T437, T438, M439, K440, A468 (Q468), H494, P496, Y497, F516, N520, and D524;
(v) a Mannheimia spp. (optionally a modified NGT of Mannheimia haemolytica of SEQ ID NO:5) comprising one or more substitutions at amino acid positions F39, R177, H214, D215, M218, H219, Y222, H272, H277, S278, 1279, R281, M349, G370, H371, T438, T439, M440, K441, A469 (Q469), H495, P497, Y498, F517, N521, and D525;
(vi) a Bibersteinia spp. (optionally a modified NGT of Bibersteinia trehalosi of SEQ ID NO:6) comprising one or more substitutions at amino acid positions F40, R180, H217, D218, M221, H222, Y225, H274, H279, S280, 1281, R283, M351, G372, H373, T440, T441, M442, K443, A471 (Q471), H497, P499, Y500, F519, N523, and D527; and
(vii) a Haemophilus spp. (optionally a modified NGT of Haemophilus ducreyi of SEQ ID NO:7) comprising one or more substitutions at amino acid positions F38, R176, H213, D214, M217, H218, Y221, H271, H276, S277, 1278, R280, M348, G369, H370, T437, T438, M439, K440, A468 (Q468), H494, P496, Y497, F516, N520, and D524.

2. The modified NGT of claim 1, wherein the amino acid substitution is at one or more positions, with reference to SEQ ID NO:1, selected from the group consisting of H219, T438, A696 and H495 or a homologous position thereof.

3. The modified NGT of claim 1, wherein the amino acid substitution is at one or more positions, with reference to SEQ ID NO:1, selected from the group consisting of: H219F or H219W; T438S or T438E; A696G or A696I; and H495D, or a homologous position thereof.

4. The modified NGT of claim 1, wherein the wild-type NGT comprises any of SEQ ID NOs:1-7 and the modified NGT comprises at least one substitution mutation, at a position with reference to SEQ ID NO:1, selected from the group consisting of: H219F, H219W, T438S, T439E, A469G, A469I, H495D, H219F-T438S, H219F-H495D, H219W-T438S, H219W-H495D, A469G-H495D, and A469I-H495D, or a homologous position thereof, wherein H219F-T438S, H219F-H495D, H219W-T438S, H219W-H495D, A469G-H495D, and A469I-H495D is a combination of two substitution mutations.

5. The modified NGT of claim 1, wherein the modified NGT glycosylates a wider array of acceptor peptide sequences as compared to an unmodified NGT under the same reaction conditions and/or wherein the modified NGT has an affinity for a wider array of acceptor peptide sequences as compared to an unmodified NGT under the same reaction conditions.

6. (canceled)

7. The modified NGT of claim 1, wherein the acceptor peptide sequence comprises the amino acid sequence [X₋₂-]-[X₋₁-]-[N]-[X₊₁]-[X₊₁]-[X₊₂]-[X₊₃], where X is any canonical amino acid, and optionally where [X₊₁] is not P.

8. The modified NGT of claim 1, wherein the acceptor peptide sequence comprises the amino acid sequence [X₋₂]-[X₋₁-]-[N]-[X₊₁]-[X₊₁]-[X₊₂]-[X₊₃], where X is any canonical amino acid, and optionally where [X₊₁] is not P, and optionally where [X₊₂] is not S or T.

9. A polynucleotide sequence encoding the modified NGT of claim 1.

10. (canceled)

11. A bacterial cell or a eukaryotic cell comprising the modified NGT of claim 1.

12. The bacterial cell or the eukaryotic cell of claim 11, further comprising a target polypeptide.

13-14. (canceled)

15. A method for glycosylating a target polypeptide, wherein the target polypeptide comprises an acceptor peptide sequence, the method comprising: contacting the target polypeptide with the modified NGT of claim 1 and a glycan under suitable reaction conditions.

16. The method of claim 15, wherein the target polypeptide comprises a therapeutic polypeptide.

17-20. (canceled)

21. The method of claim 15, wherein the target polypeptide comprises an acceptor peptide sequence comprising the amino acid sequence [X₋₂]-[X₋₁-]-[N]-[X₊₁]-[X₊₁]-[X₊₂]-[X₊₃], where X is any canonical amino acid, and optionally where [X₊₁] is not P, and optionally where [X₊₂] is not S or T.

22. The method of claim 15, wherein the NGT glycosylates the target polypeptide with one or more glycans.

23. The method of claim 15, wherein the glycan comprises one or more monosaccharides selected from the group consisting of glucose, galactose, and N-glucosamine.

24. A modified N-glycosyltransferase (NGT) comprising one or more substitutions at amino acid positions corresponding to Actinobacillus pleuropneumoniae NGT of SEQ ID NO:1: F39, R177, H214, D215, M218, H219, Y222, H272, H277, S278, 1279, R281, M349, G370, H371, T438, T439, M440, K441, A469, H495, P497, Y498, F517, N521, and D525.

25. The modified NGT of claim 24, wherein the NGT is derived from an organism selected from the group consisting of: Kingella kingae; Haemophilus influenzae; Aggregatibacter aphrophilus; Mannheimia haemolytica; Bibersteinia trehalosi; Haemophilus ducreyi; Burkholderia sp; Yersinia enterocolitia; Yersinia pestis; Salmonella enterica; and Escherichia coli.

26. The modified NGT of claim 24, wherein the NGT is derived from an organism selected from the group consisting of: Mannheimia haemolytica and Haemophilus ducreyi.

27-28. (canceled)

29. A therapeutic composition comprising the therapeutic peptide of claim 16.

30. The therapeutic composition of claim 29, wherein the composition comprises a vaccine.
